(19) United States
(12) Patent Application Publication
(10) Pub. No.: US 2024/0261416 A1
(43) Pub. Date: Aug. 8, 2024

(54) SYNTHESIS OF NOVEL CEREBLON E3 LIGASE LIGANDS, COMPOUNDS FORMED THEREBY, AND PHARMACEUTICAL COMPOSITIONS CONTAINING THEM

(71) Applicant: Wisconsin Alumni Research Foundation, Madison, WI (US)

(72) Inventors: Weiping Tang, Middleton, WI (US); Haibo Xie, Shanghai (CN); Yunxiang Wu, Madison, WI (US); Junzhuo Liao, New York, NY (US); Chunrong Li, Madison, WI (US); Ira Tandon, Madison, WI (US); Hua Tang, Madison, WI (US)

(73) Assignee: Wisconsin Alumni Research Foundation, Madison, WI (US)

(21) Appl. No.: 18/426,920

(22) Filed: Jan. 30, 2024

Related U.S. Application Data
(60) Provisional application No. 63/482,453, filed on Jan. 31, 2023.

(51) Int. Cl. A61K 47/55; C07D 239/22; C07D 401/12; C07D 403/12; C07D 403/14

(52) U.S. Cl. CPC A61K 47/55; C07D 239/22; C07D 401/12; C07D 403/12; C07D 403/14

(57) ABSTRACT

Provided herein are achiral cereblon (CRBN) ligands based on phenyl dihydrouracil that have optimal binding to CRBN without having chiral carbons. Also provided herein are the proteolysis targeting chimeras (PROTACs) comprising the CRBN ligands and a protein binder, pharmaceutical compositions containing the PROTACs, and methods of treating diseases using the PROTACs.

### CROSS-REFERENCE TO RELATED APPLICATIONS

Priority is hereby claimed to U.S. Provisional Application 63/482,453, filed Jan. 31, 2023, which is incorporated herein by reference in its entirety.

### FEDERAL FUNDING STATEMENT

This invention was made with government support under GM120357 and GM148266 awarded by the National Institutes of Health. The government has certain rights in the invention.

### BACKGROUND

Proteolysis Targeting Chimera (PROTAC, FIG. 1) technology has become an exciting therapeutic option to confront diseases involving aberrantly expressed or mutated disease-causing proteins by engaging our body's natural protein disposal system.¹⁻⁴ PROTACs comprise heterobifunctional small molecules with a linker between two motifs.⁵ The bifunctional nature in this case is defined by one end of the PROTAC binding with high selectivity to an E3 ubiquitin ligase while the other end simultaneously engages the protein of interest (POI). As the binding event occurs, the exposed lysine residues of the POI are brought in close contact with the E3 ligase complex and are poly-ubiquitinated for degradation in proteasome. The PROTAC is released to continue its catalytic activity for the degradation of the POI. Early studies used peptides as the E3 ligase ligand and the resulting PROTACs generally showed low cellular potency, partially due to their low permeability and stability.⁵⁻⁷ The recent discovery of potent small molecule E3 ligase ligands, including thalidomide analogues for cereblon (CRBN),⁸⁻¹⁰ and peptidomimetics for Von Hippel-Lindau (VHL)¹¹⁻¹³ and Inhibitor of Apoptosis Protein (IAP),¹⁴⁻¹⁶ led to the surge of a number of small molecule PROTACs with high potency in cells (e.g. nM of IC₅₀s) and impressive efficacy in animal models since 2015, such as 1 (dBET)⁸ and 2 (PROTAC_RIPK)¹¹ (FIG. 2).

Traditional occupancy-driven small molecule drug discovery approaches can only target a small portion of the proteome, because the small molecules need to bind to the functional site of the protein, such as the active site or allosteric site of enzymes. PROTACs only require a specific small molecule binder, and this allows one to target a large portion of proteome that have previously been considered “undruggable” by small molecules.¹⁷ In addition, the degradation function of PROTACs offer a number of advantages over inhibitors as therapeutics and chemical tools.¹⁸⁻²⁰ 1) The occupancy-driven pharmacology of inhibitors requires strong and stoichiometric drug binding to the POI in order to modulate the protein function. In contrast, induced protein degradation by PROTACs is event-driven and catalytic, providing favorable pharmacology. PROTACs are often effective at lower doses than inhibitors. Mutants that are resistant to inhibitors because low binding affinity may still be sensitive to degraders, because PROTACs with a weak binder can still mediate efficient degradation of the target protein. 2) The degradation mechanism can offer another level of selectivity including isoform- and tissue selectivity. For example, it has been observed previously that tethering non-selective kinase inhibitors with E3 ligase ligands can lead to selective degraders.^{20,21} A selective HDAC6 degrader based on a pan-HDAC inhibitor is also developed.²² 3) The degradation activity can negate the effects of protein overexpression mediated by the feedback mechanism, which often leads to pharmacological insufficiency of traditional drugs and drug resistance. 4) The effect may last longer because it takes the body time—often days or weeks—to replace the degraded protein, which means a less frequent dose and better patient compliance.

Although PROTAC is a relatively new technology, over a dozen of them have progressed into human clinic trials since 2019 for the degradation of oncogenic proteins and a few other targets by recruiting either VHL or CRBN E3 ligases.²³ To date, there are about ten E3 ligases have been reported to facilitate the degradation of POI by PROTACs, including IAPs, VHL, CRBN, MDM2, AhR, DCAF11, DCAF15, DCAF16, RNF4, RNF114, KEAP1, and FEMIB.^{21,24} However, only two of them, CRBN and VHL, have been widely used. According to PROTAC-DB,²⁵ an open-access database that compiles PROTAC information, out of the ˜2,200 PROTACs developed to target >120 different POIs, most of them employed two ligases: CRBN (˜1,400 PROTACs) and VHL (˜700 PROTACs). The peptide-mimetic nature of the VHL ligands makes the development of orally drug much more difficult.²⁶

CRBN is the most frequently utilized E3 ligase due to the low molecular weight and drug-like properties^{27,28} of its glutarimide ligands such as thalidomide 1A, pomalidomide 1B, and lenalidomide 1C (FIG. 3).¹⁰ These glutarimides were first applied to the development of PROTACs for targeted protein degradation in 2015.^{8,9} The first two PROTACs to progress into clinical trials (ARV-110 and ARV-471) both used glutarimide CRBN ligands.²³ However, glutarimides are used as a racemic mixture in almost all reported PROTACs with a few exceptions because the two enantiomers can undergo rapid and spontaneous racemization in vitro and in vivo. For example, the racemization half-life of thalidomide is ˜2.0 h in human blood and ˜5.0 h in-vivo in humans, respectively.²⁹ The binding affinity of the (S)-enantiomeric thalidomide to CRBN is at least 10-fold stronger than the corresponding (R)-enantiomer.^{30,31} The DDB1-CRBN-thalidomide complex further confirms that only the (S)-enantiomer fits the binding pocket well. The glutarimide moiety is most critical for binding as three hydrogen bonds are formed between the imide motif and CRBN, whereas the phthalimide or the phthalimidine moiety is exposed to the solvent.³²

The use of racemic glutarimide as the E3 ligase ligands in PROTACs complicates many aspects of the drug development process and is one of the significant barriers for the therapeutic applications of CRBN-recruiting PROTACs. For example, half of the PROTAC molecules that have poor degradation activity due to ˜ 10-fold weaker binding affinity to CRBN can competitively inhibit the other half of the active portion, because they bear the same POI ligand that can bind to the protein target. In addition, according to the FDA policy updated in 1992, more resources are needed to characterize the pharmacological properties of all isomers including enantiomers and their metabolites during the drug development process.³³ To date, various CRBN ligands have been reported by researchers from both industry and academia; some of these reports include efforts to solve the chirality issue.³⁴⁻³⁸ For example, the racemization of deuterated thalidomide 1D (FIG. 3), where the hydrogen is replaced by a deuterium, was about five times slower than thalidomide.³⁹ If an all carbon quaternary center was to be introduced, no racemization would occur. However, when the Y substituent in 1 was replaced by a methyl group, the corresponding compound 1E lost its activity.^{40,41} In addition to racemization, stability of the glutarimides is another issue. To address the issues of existing CRBN ligands, the present disclosure designs a novel class of achiral CRBN ligands for the development of non-racemic PROTACs.

### SUMMARY

Disclosed herein is class of achiral cereblon (CRBN) ligands derived from phenyl dihydrouracil (PDHU) that have optimal binding to CRBN without having chiral carbons. In some embodiments, the CRBN ligands are bi- or tri-substituted PDHU molecules.

Specifically disclosed herein are compounds having the following structure:

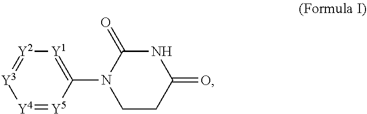

wherein:
Y¹, Y², Y³, Y⁴, and Y⁵ are each independently CH, CR, CR¹, or N, with the proviso that at least one of Y¹, Y², Y³, Y⁴, and Y⁵ is CR;
R¹ in each instance is independently C1-C3 alkyl, halogen-substituted C1-C3 alkyl, C1-C3 alkyloxy, or halogen-substituted C1-C3 alkyloxy; and

R in each instance is independently hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments, Y¹ and Y³ are each independently CH, CR, CR¹, or N, and Y², Y⁴, and Y⁵ are each independently CH, CR, CR¹.

In some embodiments, Y¹ is CH or CR¹; Y², Y³ and Y⁴ are each independently CH or CR; and Y⁵ is CH.

In some embodiments, Y¹ is CH or CR¹, Y² and Y⁴ are each independently CH or CR, and Y³ and Y⁵ is CH.

In some embodiments, Y¹ is CR¹.

In some embodiments, Y² is CR.

In some embodiments, Y⁴ is CR.

In some embodiments, at least one of Y¹, Y², Y³, Y⁴, and Y⁵ is CH. In some embodiments, at least two of Y¹, Y², Y³, Y⁴, and Y⁵ are CH. In some embodiments, at least three of Y¹, Y², Y³, Y⁴, and Y⁵ are CH. In some embodiments, at least four of Y¹, Y², Y³, Y⁴, and Y⁵ are CH. In some embodiments, Y¹, is CH. In some embodiments, Y² is CH. In some embodiments, Y³ is CH. In some embodiments, Y⁴ is CH. In some embodiments, Y⁵ is CH.

In some embodiments, at least one of Y¹, Y², Y³, Y⁴, and Y⁵ is CR. In some embodiments, at least two of Y¹, Y², Y³, Y⁴, and Y⁵ are CR. In some embodiments, at least three of Y¹, Y², Y³, Y⁴, and Y⁵ are CR. In some embodiments, at least four of Y¹, Y², Y³, Y⁴, and Y⁵ are CR. In some embodiments, Y¹, is CR. In some embodiments, Y² is CR. In some embodiments, Y³ is CR. In some embodiments, Y⁴ is CR. In some embodiments, Y⁵ is CR.

In some embodiments, at least one of Y¹, Y², Y³, Y⁴, and Y⁵ is CR¹. In some embodiments, at least two of Y¹, Y², Y³, Y⁴, and Y⁵ are CR¹. In some embodiments, at least three of Y¹, Y², Y³, Y⁴, and Y⁵ are CR¹. In some embodiments, at least four of Y¹, Y², Y³, Y⁴, and Y⁵ are CR¹. In some embodiments, Y¹, is CR¹. In some embodiments, Y² is CR¹. In some embodiments, Y³ is CR¹. In some embodiments, Y⁴ is CR¹. In some embodiments, Y⁵ is CR¹.

In some embodiments:
R is:

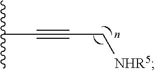

n is 1-4;
R⁵ is an amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is:

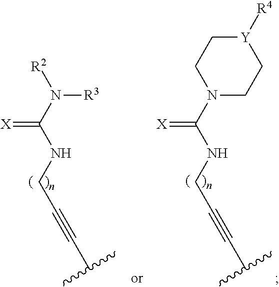

n is 1-4;
X is O or S;
R² is R^{A};
R³ is an amino protecting group or R^{A};
Y is N or CH;
R⁴ is amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is —X—R^{A};
X is —O—, NH, or —C(O)—; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is:

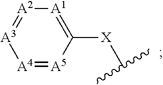

X is —O—, NH, or —C(O)—;
A¹, A², A³, A⁴, and A⁵ are each independently CH, CE, CR^{A}, or N, with the proviso that at least one of A¹, A², A³, A⁴, and A⁵ is CR^{A};
E is halogen, halogen-substituted alkyl, alkyloxy, alkenyloxy, alkynyloxy, cycloalkyloxy, cycloalkenyloxy, alkylsulfonyloxy, cycloalkenylsulfonyloxy, amino, protected amino, acyl, formyl, alkyloxycarbonyl, alkenyloxycarbonyl, alkynyloxycarbonyl, aryloxycarbonyl, cyano, nitro, aryloxy, heteroaryloxy, heteroarylsulfonyloxy, non-aromatic heterocycle, or any combination of the forgoing; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments, one or two of A¹, A², A³, A⁴, and A⁵ is N.

In some embodiments, at least one of A², A³, and A⁴ is CR^{A} and at least one of A², A³, and A⁴ is CE.

In some embodiments, A¹ and A⁵ are each CH.

In some embodiments, at least one of A¹, A², A³, A⁴, and A⁵ is CR^{A}. In some embodiments, at least two of A¹, A², A³, A⁴, and A⁵ are CR^{A}. In some embodiments, at least three of A¹, A², A³, A⁴, and A⁵ are CR^{A}. In some embodiments, at least four of A¹, A², A³, A⁴, and A⁵ are CR^{A}. In some embodiments, A¹, is CR^{A}. In some embodiments, A² is CR^{A}. In some embodiments, A³ is CR^{A}. In some embodiments, A⁴ is CR^{A}. In some embodiments, A⁵ is CR^{A}.

In some embodiments, at least one of A¹, A², A³, A⁴, and A⁵ is CE. In some embodiments, at least two of A¹, A², A³, A⁴, and A⁵ are CE. In some embodiments, at least three of A¹, A², A³, A⁴, and A⁵ are CE. In some embodiments, at least four of A¹, A², A³, A⁴, and A⁵ are CE. In some embodiments, A¹, is CE. In some embodiments, A² is CE. In some embodiments, A³ is CE. In some embodiments, A⁴ is CE. In some embodiments, A⁵ is CE.

In some embodiments, at least one of A¹, A², A³, A⁴, and A⁵ is CH. In some embodiments, at least two of A¹, A², A³, A⁴, and A⁵ are CH. In some embodiments, at least three of A¹, A², A³, A⁴, and A⁵ are CH. In some embodiments, at least four of A¹, A², A³, A⁴, and A⁵ are CH. In some embodiments, A¹, is CH. In some embodiments, A² is CH. In some embodiments, A³ is CH. In some embodiments, A⁴ is CH. In some embodiments, A⁵ is CH.

In some embodiments. R is selected from:

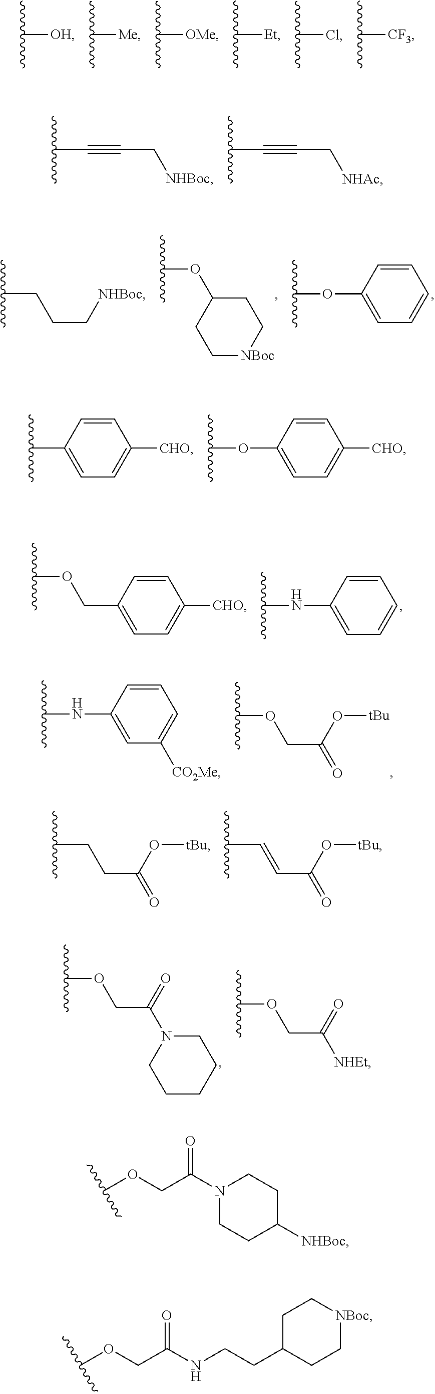

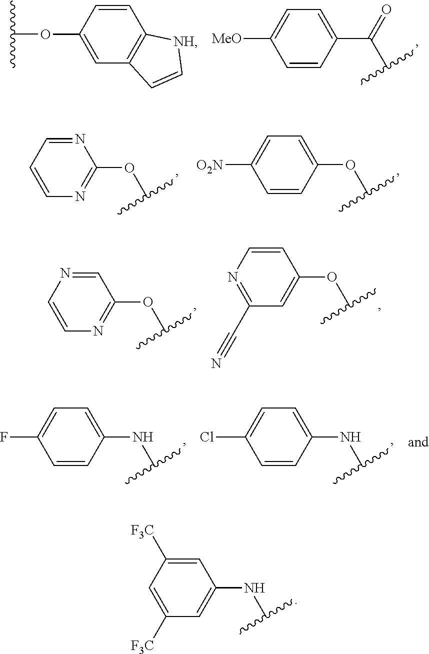

In some embodiments, the compound is selected from:

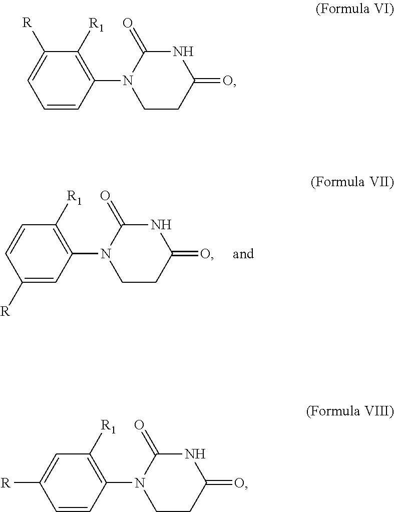

wherein R₁ in each instance is independently Me, Et, OMc, or CF₃.

In Formulas VI, VII and VIII, when R₁ is Me, the compound in some embodiments is selected from:

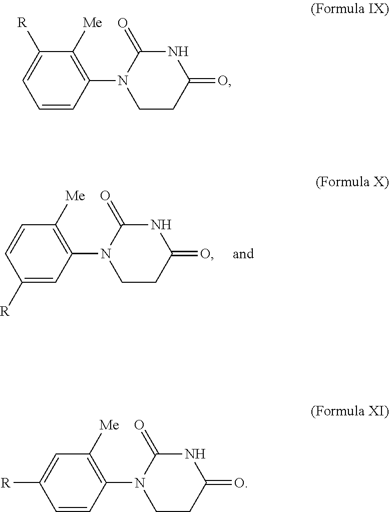

In some embodiments of Formula VI, the compound is selected from:

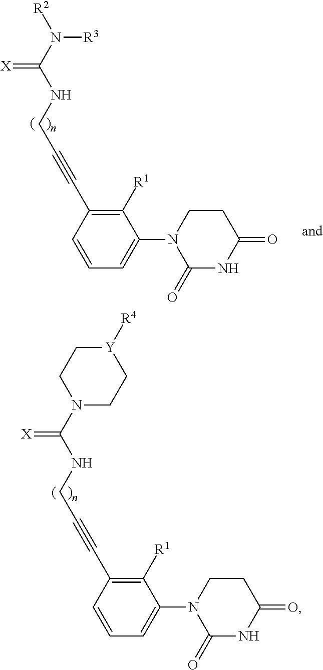

wherein:
R¹ in each instance is independently Me, Et, or CF₃;
R² and R³ in each instance are independently hydrogen or hydrophobic groups including rings;
X in each instance is independently O or S;
Y in each instance is independently N or CH;
n is 1-4; and
R⁴ is hydrogen, hydroxy, alkyl, alkenyl, alkynyl, alkyloxy, amino, acyl, formyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, or heteroarylsulfonyloxy.

In some embodiments of Formula VII, the compound is selected from:

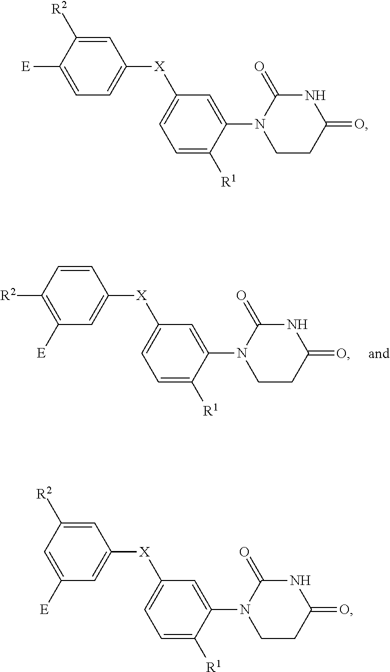

wherein:
R¹ in each instance is independently Me, Et, OMe, or CF₃;
R² in each instance is independently alkynyl or alkyl;
E in each instance is independently CN, ketone, ester, or amide; and
X in each instance is independently O, NH, or carbonyl.

In some embodiments, the compound is selected from:

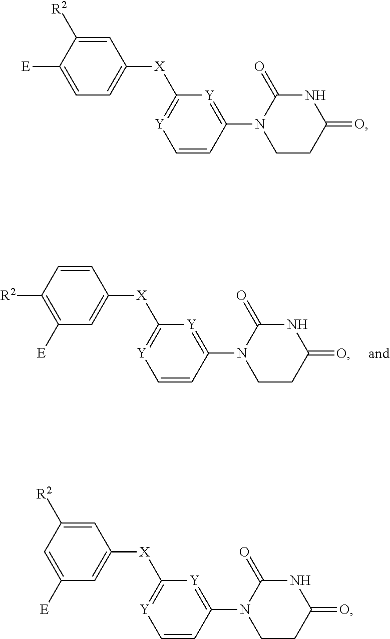

wherein:
R² in each instance is independently alkynyl or alkyl;
E in each instance is independently CN, ketone, ester, or amide;
X in each instance is O, NH, or carbonyl; and
Y in each instance is independently N or CH.

Also disclosed herein is a proteolysis targeting chimera (PROTAC) comprising the compound that binds to CRBN according to the present disclosure and a protein binder that binds a target protein. In certain versions, the protein binder is a polypeptide, a ligand, an aptamer, a nanoparticle, or a small molecule. The PROTAC may further comprise a linker connecting the CRBN-binding compound and the protein binder.

Also disclosed herein are pharmaceutical compositions comprising any of the PROTAC according to the present disclosure.

Also disclosed herein is a method of degrading a target protein, comprising contacting the target protein with the PROTAC as described herein, wherein the PROTAC mediates degradation of the target protein in proteasome.

Also disclosed herein is a method that includes administering to an individual in need thereof a therapeutically effective amount of any of the pharmaceutical compositions of the present disclosure.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1. Illustration of PROTAC-induced degradation of protein of interest (POI) via ubiquitin-proteasome-system.

FIG. 2. Selected PROTACs that demonstrate in-vivo activity.

FIG. 3. Various glutarimide.

FIG. 4. Comparison of the well-known glutarimide and achiral dihydrouracil.

FIG. 5. Relative binding affinity of substituted PDHUs in a FP assay lenalidomide (1C) was used as the positive control and each compound was tested 3 times).

FIG. 6. K_{d} values of substituted PDHUs.

FIG. 7. BRD4 degraders with achiral CRBN ligands

FIGS. 8A-8H. (FIG. 8A) Western blot of BRD4. MV4; 11, RS4; 11, MOLT4 and LNCaP cells were treated by 12A, 12B, 12C, 13 and 14 at 1.0 and 0.1 μM for 24 h. (FIG. 8B) Time course based on Western blot of BRD4, caspase 3 and cleaved caspase 3. MV4; 11 cells were treated by 13 with indicated time. (FIG. 8C) and (FIG. 8D) Dose responses based on Western blot of BRD4, caspase 3 and cleaved caspase 3. MV4; 11 cells were treated by 13 with indicated concentrations for 24 h. DC₅₀: the concentration where 50% of the protein has been degraded. Dₘₐₓ: the maximum degradation that can be achieved. (FIG. 8E) Quantitative assessment of degradation using a BRD4_{BDI}-e-GFP reporter assay. Dose response based on fluorescent signal ratio of e-GFP to mCherry. Flp-In™-293 Cell Line was treated with 13 at indicated concentrations for 24 h. (FIG. 8F), (FIG. 8G) and (FIG. 8H) Confirmation of mechanism of action by Western blot of BRD4. For (FIG. 8F) and (FIG. 8G), MV4; 11 cells were pretreated by JQ1, 9, MG132 or MLN4924 for 1 h followed by the treatment of 13 for 3 h. For (FIG. 8H), MV4; 11 cells were treated by 13NT at indicated concentrations for 24 h.

FIGS. 9A-9D. Functional studies of the PROTACs including anti-proliferation, apoptosis, G1 phase arrest, and reduced population in S phase. (FIG. 9A) AlamarBlue™ Cell Viability. MV4; 11 cells were treated by 13, 9, (+)-JQ1 and 13NT for 72 h. (FIG. 9B) MV4; 11 cells were treated by indicated dosage of 13 for 48 h followed by flow cytofluorimetric cell cycle analysis. (FIGS. 9C and 9D) MV4; 11 cells were treated by indicated dosage of 13 for 48 h followed by flow cytofluorimetric apoptosis analyses.

FIG. 10. Binding affinity of selected PDHUs in the FP assay (% is for single concentration).

FIG. 11. Compounds based on 6F.

FIG. 12. Compounds based on 15A.

FIG. 13. Compounds similar to 15 A and relative binding affinities compared to lenalidomide (1C), as performed in FIG. 5 (percentages for each replicate shown).

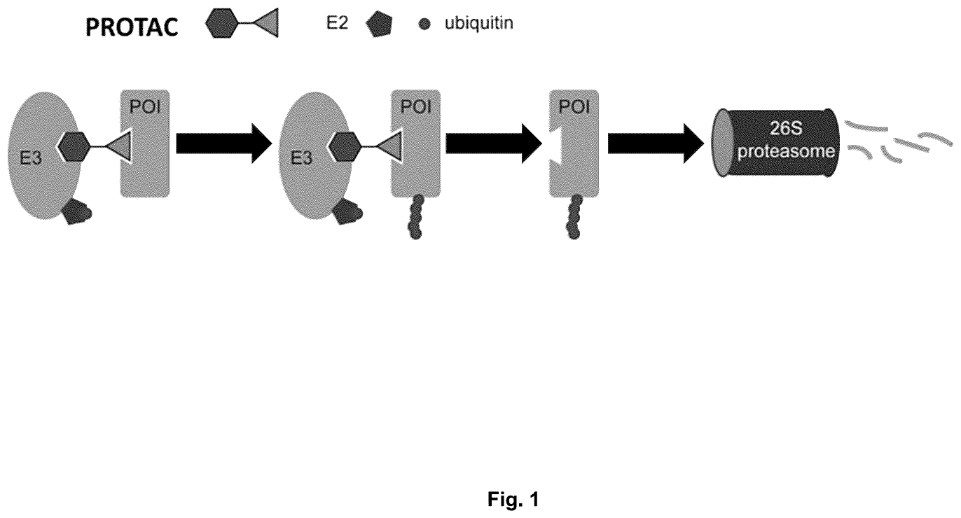

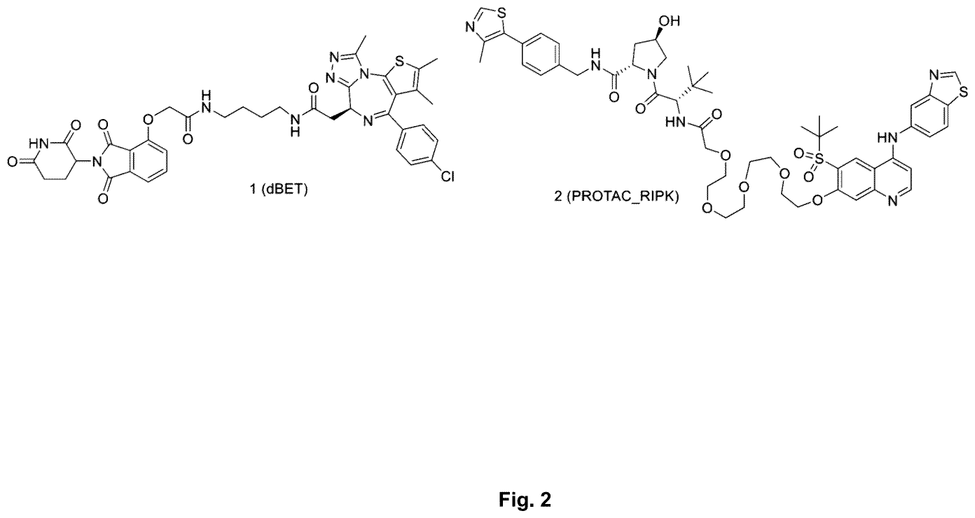

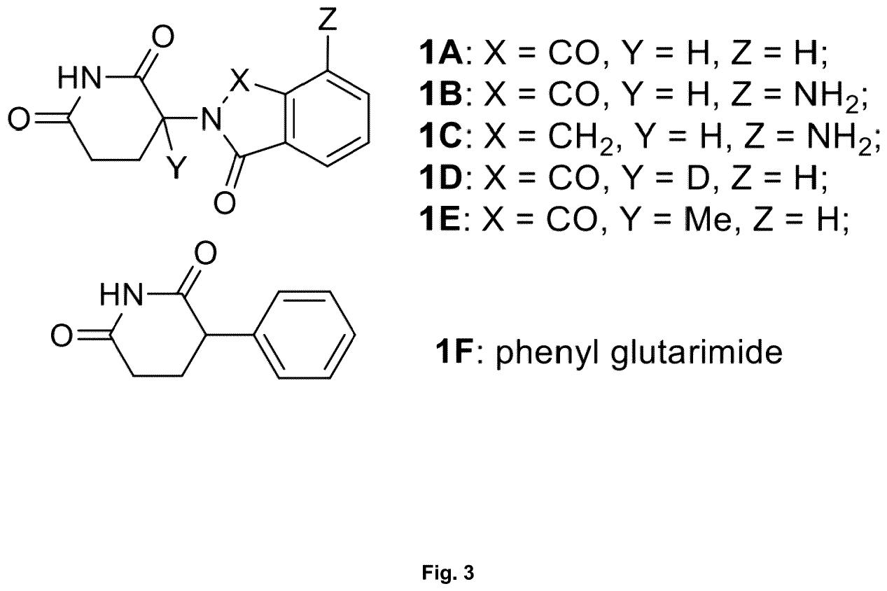

### DETAILED DESCRIPTION

### Definitions and Abbreviations

CRBN=Cereblon; PDHU=Phenyl Dihydrouracil;

POI=Protein of Interest; PROTAC=Proteolysis Targeting Chimera.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

As used herein, the term “about” refers to +10% of the variable referenced.

As used herein, the singular forms “a,” “an,” and “the” include plural referents unless the content clearly dictates otherwise.

As used herein, the term “or” is an inclusive “or” operator and is equivalent to the term “and/or” unless the context clearly dictates otherwise.

The elements and method steps described herein can be used in any combination whether explicitly described or not, unless otherwise specified or clearly implied to the contrary by the context in which the referenced combination is made.

All combinations of method steps as used herein can be performed in any order, unless otherwise specified or clearly implied to the contrary by the context in which the referenced combination is made.

The system disclosed herein my comprise, consist of, or consist essentially of the various steps and elements disclosed herein.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls. Xie et al. 2023 (Xie H, Li C, Tang H, Tandon I, Liao J, Roberts B L, Zhao Y, Tang W. Development of Substituted Phenyl Dihydrouracil as the Novel Achiral Cereblon Ligands for Targeted Protein Degradation. J Med Chem. 2023 Feb. 23; 66(4):2904-2917) forms part of the present disclosure and is incorporated by reference in its entirety.

It is understood that the disclosure is not confined to the particular elements and method steps herein illustrated and described, but embraces such modified forms thereof as come within the scope of the claims.

### Achiral CRBN Ligands

Provided herein is a class of achiral molecules that bind cereblon (CRBN), a ubiquitin ligase. These molecules may be used in the development of PROTAC therapeutics. The class of achiral CRBN ligands is based on phenyl dihydrouracil (PDHU) that have optimal binding to cereblon without having chiral carbons.

In some embodiments, the achiral CRBN ligands of the invention have the following structure:

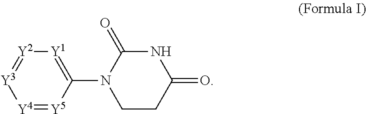

In some embodiments, Y¹, Y², Y³, Y⁴, and Y⁵ are each independently CH, CR, CR¹, or N, with the proviso that at least one of Y¹, Y², Y³, Y⁴, and Y⁵ is CR.

In some embodiments, R¹ in each instance is independently C1-C3 alkyl, halogen-substituted C1-C3 alkyl, C1-C3 alkyloxy, halogen-substituted C1-C3 alkyloxy, or any combination of the foregoing.

In some embodiments, R in each instance is independently hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkenylsulfinyl, cycloalkenylsulfonyl, cycloalkylsulfonyloxy, cycloalkenylthio, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

Definitions for the above mentioned R moieties (hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, and non-aromatic heterocycle (also known as a non-aromatic heterocyclic group)), examples thereof, and optional substitutions on same (e.g., as would occur with various combinations of the foregoing) can be found in U.S. Pat. No. 11,548,885 B2, which is incorporated herein by reference in its entirety. In case of conflict between U.S. Pat. No. 11,548,885 B2 and any descriptions herein, the present disclosure controls. “Protected amino” refers to an amino group with at least one amino protecting group. Exemplary amino protecting groups include 9-fluorenylmethyl carbamate (FMOC), t-butyl carbamate (BOC), benzyl carbamade (Cbz), acetamide (Ac), trifluoroacetamide, phthalimide, benzylamine (Bn), triphenylmethylamine (tritylamine) (Tr), benzylideneamine, and p-toluenesulfonyl (tosyl), among others.

The recited R moieties (hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, and non-aromatic heterocycle groups) are referred to herein in their monovalent radical forms for convenience only and can be present in any given compound of the invention in multivalent radical form (e.g., bivalent or trivalent radical forms), where possible. The R moieties can take the multivalent forms, for example, when present as internal groups rather than terminal groups in a given embodiment, such as when two or more of the R moieties are combined with each other or to a protein binder either directly or via a linker, as described below. In some embodiments, a given R moiety can be in a multivalent radical form with the loss of one or more hydrogen atoms. For example, the recited —OH group will be present as an —O— group in certain embodiments, the recited amino group will be present as a secondary or tertiary amine group in certain embodiments, the recited acyl group will be present as a carbonyl group in certain embodiments, etc. Thus, the disclosure and recitation of the various R moieties also encompasses the disclosure and recitation, unless otherwise specified or the context dictates otherwise, of their multivalent forms such as —O— for hydroxyl, alkylene for alkyl, alkenylene for alkenyl, alkynylene for alkynyl, cycloalkylene for cycloalkyl, cycloalkenylene for cycloalkenyl, carbonyl for formyl, etc. The same considerations apply to the R¹ moieties, the E (electron withdrawing) moieties, and the R^{A} moieties as described elsewhere herein.

In some embodiments, Y¹ and Y³ are each independently CH, CR, CR¹, or N, and Y², Y⁴, and Y⁵ are each independently CH, CR, or CR¹.

In some embodiments, Y¹ is CH or CR¹; Y², Y³ and Y⁴ are each independently CH or CR;

and Y⁵ is CH.

In some embodiments, Y¹ is CH or CR¹, Y² and Y⁴ are each independently CH or CR, and Y³ and Y⁵ is CH.

In some embodiments, Y¹ is CR¹.

In some embodiments, Y² is CR.

In some embodiments, Y⁴ is CR.

In some embodiments:

R is:

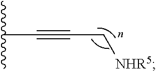

n is 1-4;
R⁵ is an amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is:

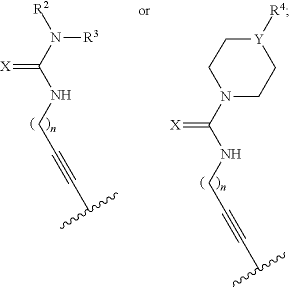

n is 1-4;
X is O or S;
R² is R^{A};
R³ is an amino protecting group or R^{A};
Y is N or CH;
R⁴ is amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is —X—R^{A};
X is —O—, NH, or —C(O)—; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments:
R is:

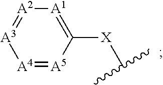

X is —O—, NH, or —C(O)—;
A¹, A², A³, A⁴, and A⁵ are each independently CH, CE, CR^{A}, or N, with the proviso that at least one of A¹, A², A³, A⁴, and A⁵ is CR^{A};
E is an electronic withdrawing group such as halogen, halogen-substituted alkyl, alkyloxy, alkenyloxy, alkynyloxy, cycloalkyloxy, cycloalkenyloxy, alkylsulfonyloxy, cycloalkenylsulfonyloxy, amino, protected amino, acyl, formyl, alkyloxycarbonyl, alkenyloxycarbonyl, alkynyloxycarbonyl, aryloxycarbonyl, cyano, nitro, aryloxy, heteroaryloxy, heteroarylsulfonyloxy, non-aromatic heterocycle, or any combination of the forgoing, among others; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

In some embodiments, one or two of A¹, A², A³, A⁴, and A⁵ is N.

In some embodiments, at least one of A², A³, and A⁴ is CR^{A} and at least one of A², A³, and A⁴ is CE.

In some embodiments. A¹ and A⁵ are each CH.

In some embodiments. R is selected from:

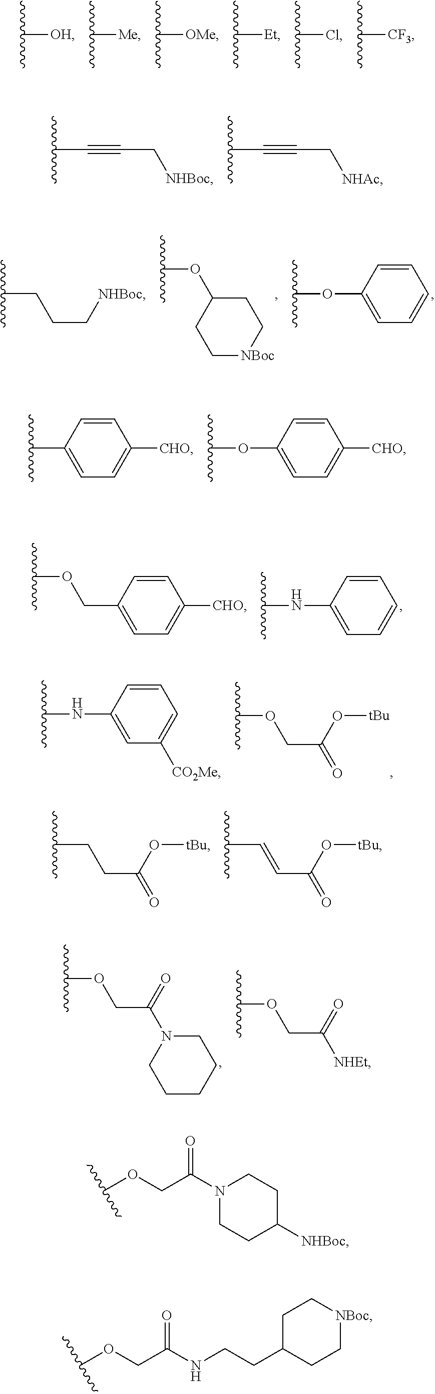

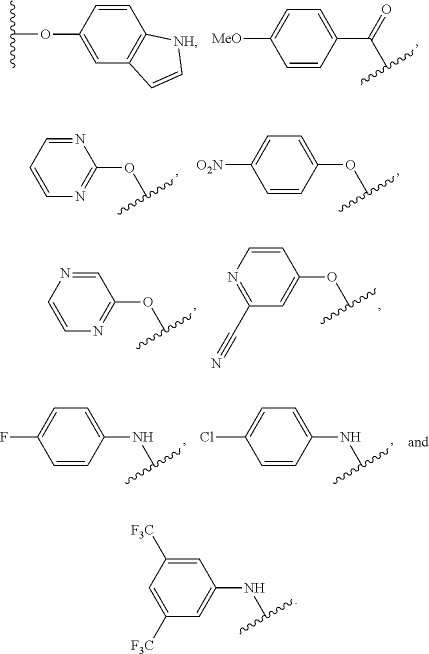

In some embodiments, the achiral CRBN ligand is selected from compounds having the following structures:

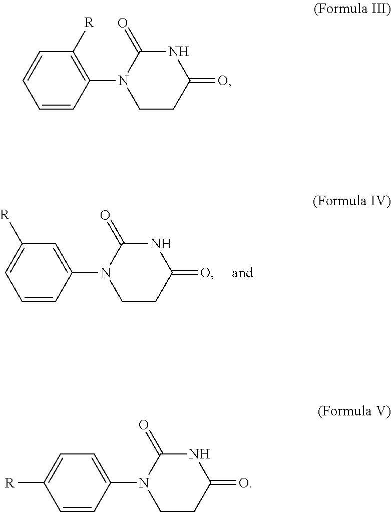

Examples of the achiral CRBN ligand having the structures of Formulas III, IV and V include:

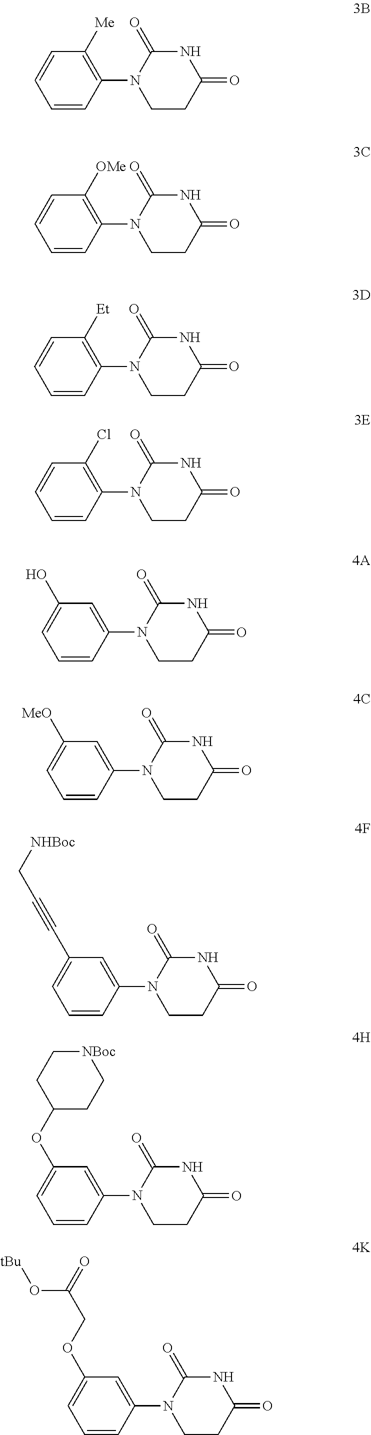

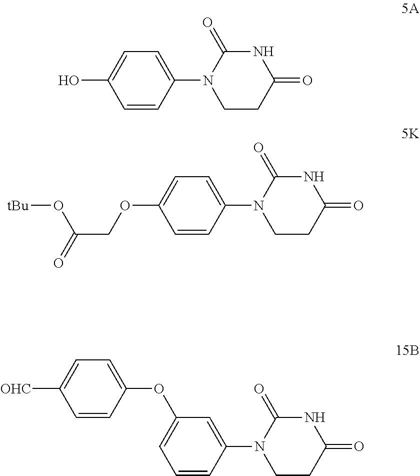

In some embodiments, the achiral CRBN ligand is selected from compounds having the following structures:

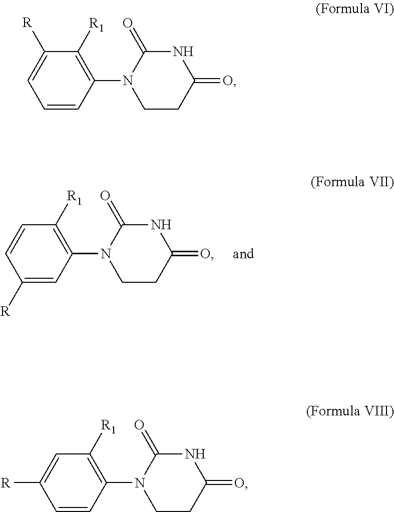

wherein R₁ is a small hydrophobic group such as Me, Et, OMe, and CF₃.

In some embodiments, when R₁ is Me, the achiral CRBN ligand is selected from compounds having the following structures:

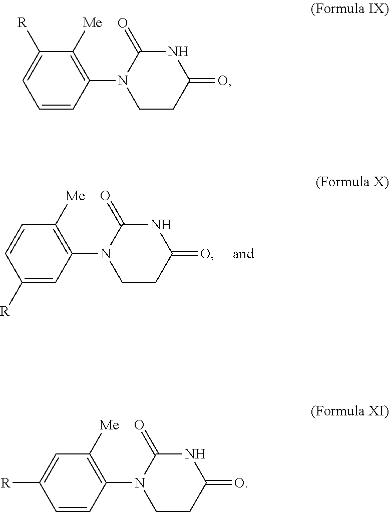

Examples of the achiral CRBN ligands include:

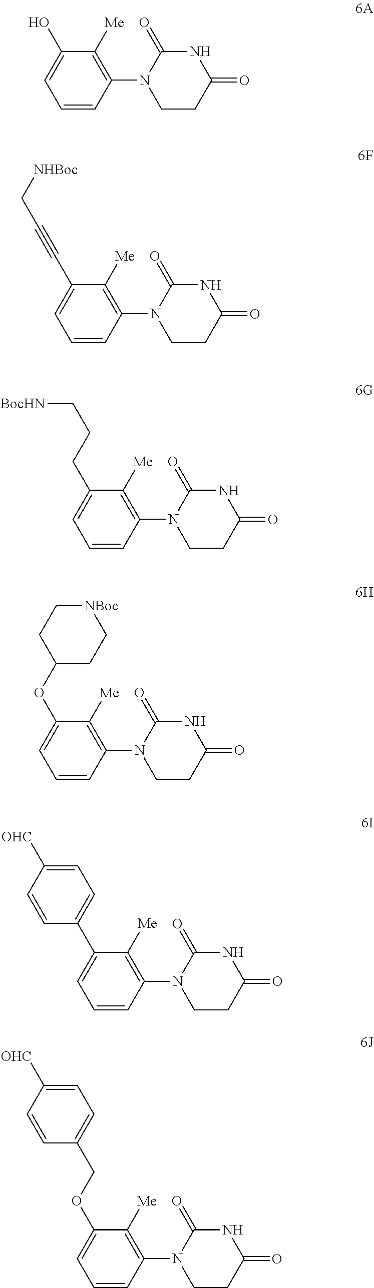 [SMILES: Cc1c(O)cccc1N1CCC(=O)NC1=O ; Cc1c(C#CCNC(=O)OC(C)(C)C)cccc1N1CCC(=O)NC1=O ; Cc1c(CCCNC(=O)OC(C)(C)C)cccc1N1CCC(=O)NC1=O ; Cc1c(OC2CCN(C(=O)OC(C)(C)C)CC2)cccc1N1CCC(=O)NC1=O ; Cc1c(-c2ccc(C=O)cc2)cccc1N1CCC(=O)NC1=O ; Cc1c(OCc2ccc(C=O)cc2)cccc1N1CCC(=O)NC1=O]

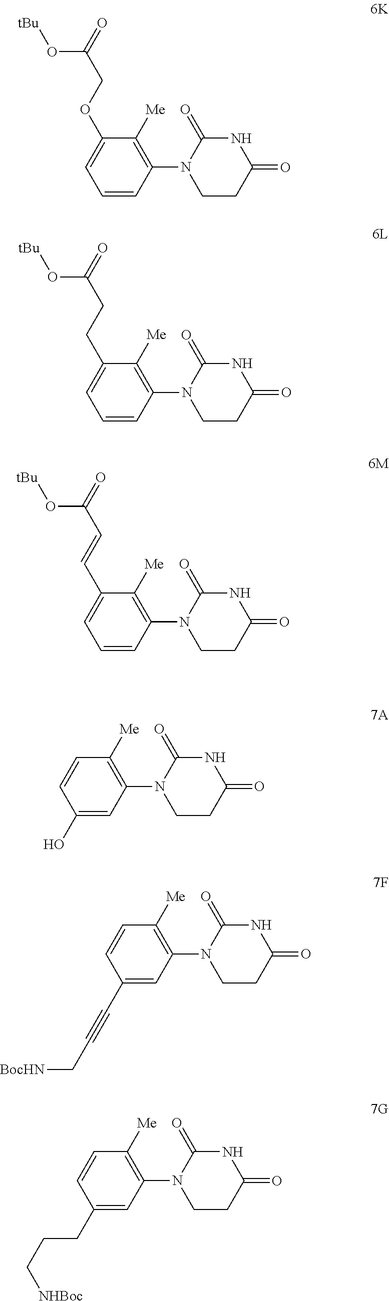 [SMILES: Cc1c(OCC(=O)OC(C)(C)C)cccc1N1CCC(=O)NC1=O ; Cc1c(CCC(=O)OC(C)(C)C)cccc1N1CCC(=O)NC1=O ; Cc1c(/C=C/C(=O)OC(C)(C)C)cccc1N1CCC(=O)NC1=O ; Cc1ccc(O)cc1N1CCC(=O)NC1=O ; Cc1ccc(C#CCNC(=O)OC(C)(C)C)cc1N1CCC(=O)NC1=O ; Cc1ccc(CCCNC(=O)OC(C)(C)C)cc1N1CCC(=O)NC1=O]

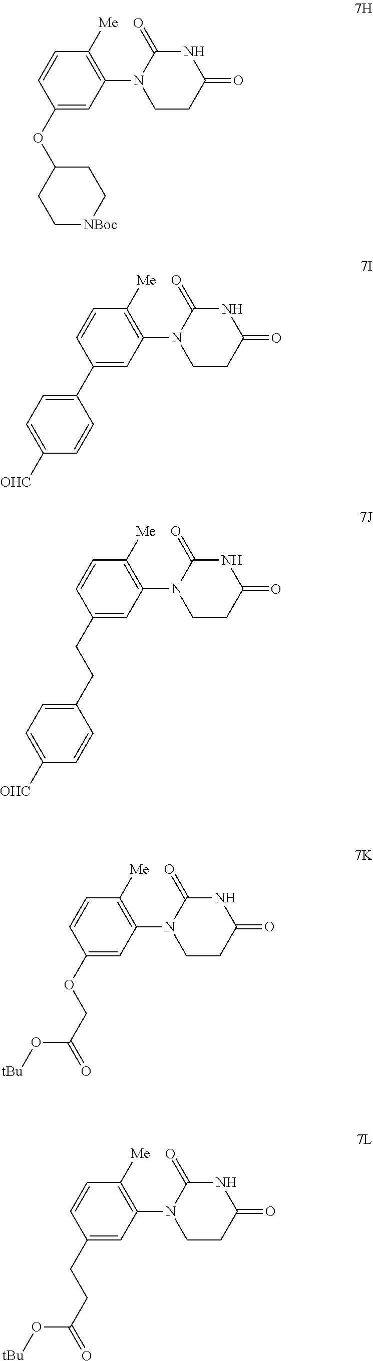 [SMILES: Cc1ccc(OC2CCN(C(=O)OC(C)(C)C)CC2)cc1N1CCC(=O)NC1=O ; Cc1ccc(-c2ccc(C=O)cc2)cc1N1CCC(=O)NC1=O ; Cc1ccc(CCc2ccc(C=O)cc2)cc1N1CCC(=O)NC1=O ; Cc1ccc(OCC(=O)OC(C)(C)C)cc1N1CCC(=O)NC1=O ; Cc1ccc(CCC(=O)OC(C)(C)C)cc1N1CCC(=O)NC1=O]

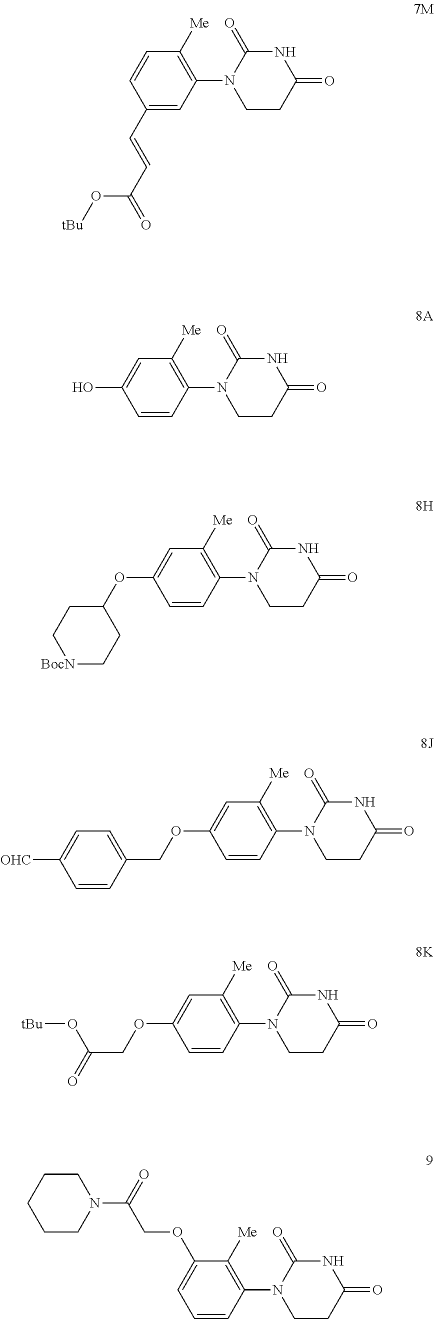 [SMILES: Cc1ccc(/C=C/C(=O)OC(C)(C)C)cc1N1CCC(=O)NC1=O ; Cc1cc(O)ccc1N1CCC(=O)NC1=O ; Cc1cc(OC2CCN(C(=O)OC(C)(C)C)CC2)ccc1N1CCC(=O)NC1=O ; Cc1cc(OCc2ccc(C=O)cc2)ccc1N1CCC(=O)NC1=O ; Cc1cc(OCC(=O)OC(C)(C)C)ccc1N1CCC(=O)NC1=O ; Cc1c(OCC(=O)N2CCCCC2)cccc1N1CCC(=O)NC1=O]

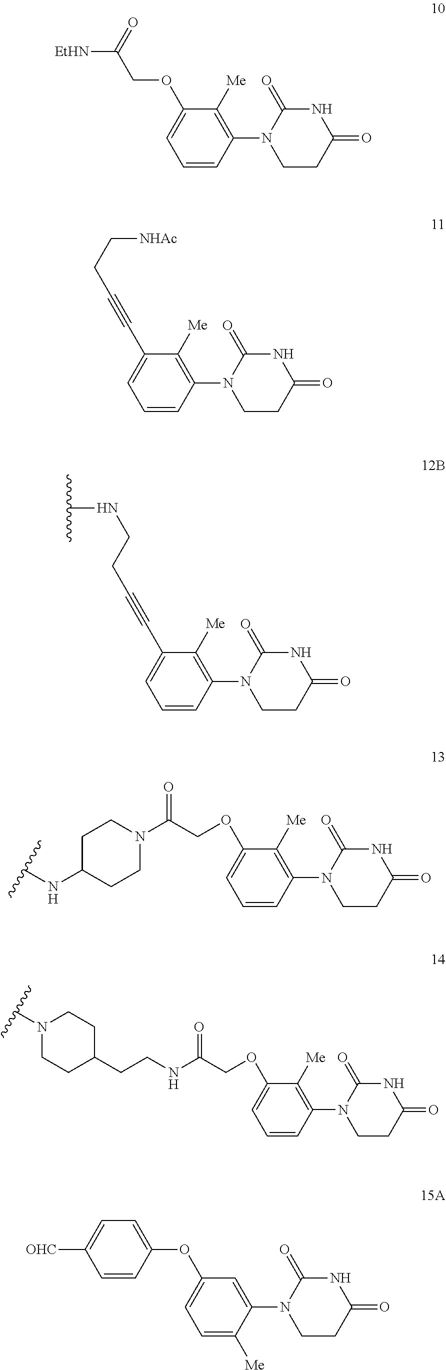

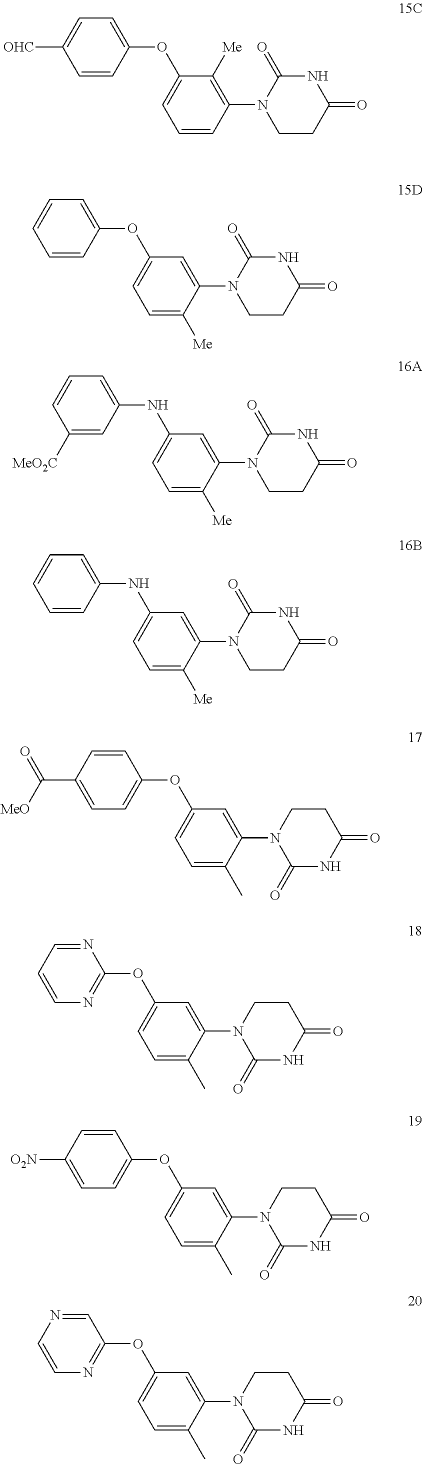

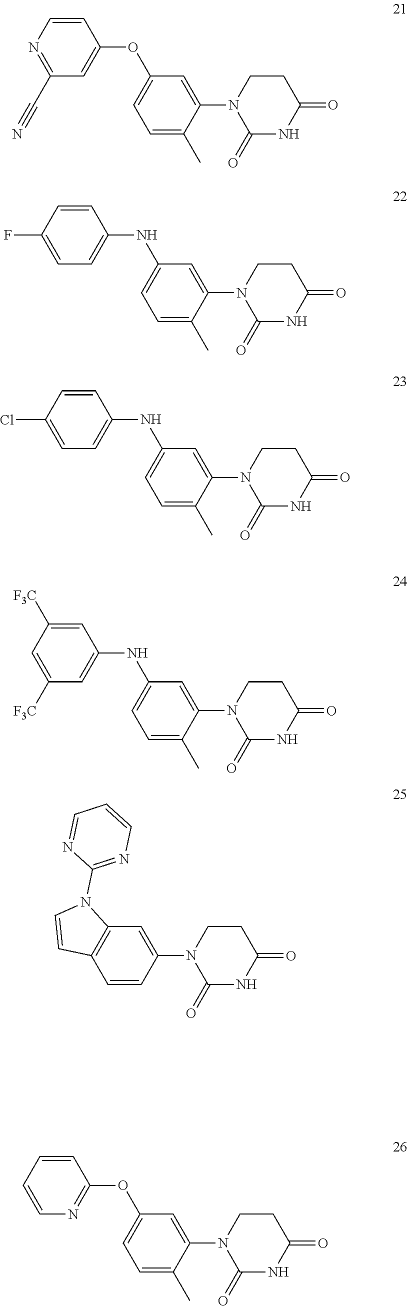 [SMILES: Cc1ccc(Oc2ccnc(C#N)c2)cc1N1CCC(=O)NC1=O ; Cc1ccc(Nc2ccc(F)cc2)cc1N1CCC(=O)NC1=O ; Cc1ccc(Nc2ccc(Cl)cc2)cc1N1CCC(=O)NC1=O ; Cc1ccc(Nc2cc(C(F)(F)F)cc(C(F)(F)F)c2)cc1N1CCC(=O)NC1=O ; O=C1CCN(c2ccc3ccn(-c4ncccn4)c3c2)C(=O)N1 ; Cc1ccc(Oc2ccccn2)cc1N1CCC(=O)NC1=O]

In some embodiments, the achiral CRBN ligand is selected from compounds having the following structures:

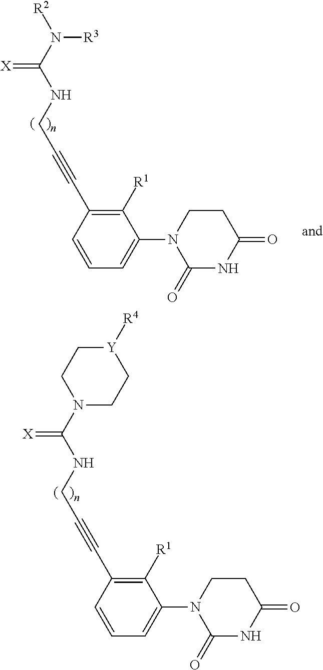

wherein R¹=small hydrophobic groups such as Me, Et, CF₃;
R² or R³═H or larger hydrophobic groups including rings;
X═O or S;
Y═N or CH;
n=1-4;
R⁴=linkers for PROTACs.

In some embodiments, the achiral CRBN ligand is selected from compounds having the following structures:

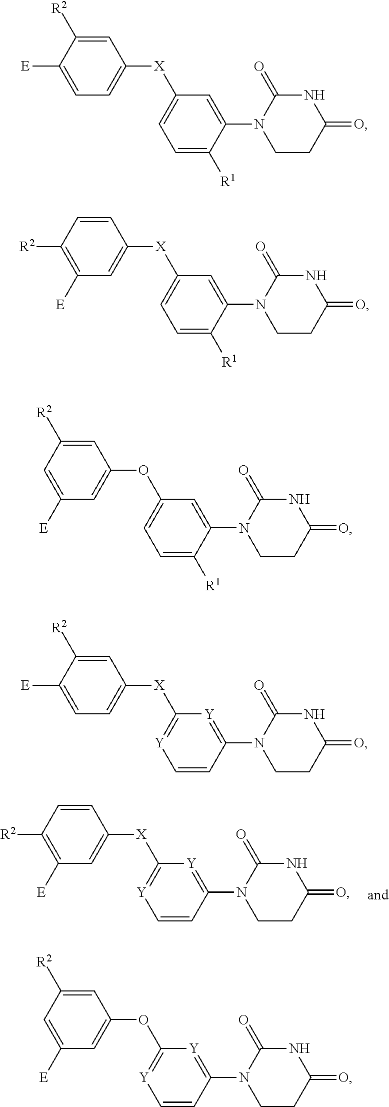

wherein R¹=small hydrophobic groups such as Me, Et, OMe, CF₃;
R²=linkers such as alkynyl or alkyl;
E=electron-withdrawing groups such as CN, ketone, ester, amide;
X═O or NH, or carbonyl;
Y═N or CH.

In some embodiments, the achiral CRBN ligand has the following structure:

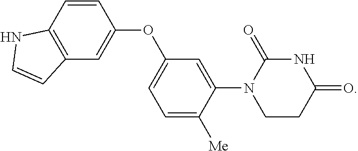 [SMILES: Cc1ccc(Oc2ccc3[nH]ccc3c2)cc1N1CCC(=O)NC1=O]

While specific embodiments disclosed herein have been shown and described in detail to illustrate the application of the principles stated above, it will be understood that the disclosure may be embodied otherwise without departing from such principles.

### Non-Racemic PROTACs

Disclosed herein are bifunctional proteolysis targeting chimera (PROTAC) molecules that include: (a) any of the achiral molecules as disclosed herein that binds CRBN; and (b) a protein binder that binds a protein of interest (POI). In some embodiments, the achiral molecule is configured to specifically bind CRBN. The protein binder is configured to specifically bind the POI. The PROTAC molecules disclosed herein finds use, e.g., for selectively targeted degradation of aberrantly expressed or mutated disease-causing proteins by engaging the ubiquitin-proteasome-system. The PROTAC binds the target protein recruiting the ubiquitin ligase (i.e., CRBN), to the target protein. The ligase adds ubiquitin to the target protein tagging it for degradation by the proteasome.

As disclosed herein, the POI is any amino acid sequence to which the protein binder of the PROTAC molecule can bind and the degradation of the POI causes a beneficial therapeutic effect in vivo.

In some embodiments, the POI is expressed from a gene that has undergone an amplification, translocation, deletion, or inversion event which causes or is caused by a medical disorder. The POI may be post-translationally modified by one, or a combination, of phosphorylation, acetylation, acylation including propionylation and crotylation, N-linked glycosylation, amidation, hydroxylation, methylation and poly-methylation, O-linked glycosylation, pyrogultamoylation, myristoylation, famesylation, geranylgeranylation, ubiquitination, sumoylation, or sulfation which causes or is caused by a medical disorder

The POI can be a non-endogenous peptide such as that from a pathogen (e.g., virus, bacteria, or fungus) or toxin. Alternatively, the POI can be an endogenous protein that mediates a disorder. The endogenous protein can be either the normal form of the protein or an aberrant form. For example, the POI can be a mutant protein found in cancer cells, or a protein, for example, where a partial, or full, gain-of-function or loss-of-function is encoded by nucleotide polymorphisms.

Nonlimiting examples of the POI include a protein that mediate an epigenetic action such as DNA methylation or covalent modification of histones (e.g., histone deacetylase), bromodomain, structural protein, receptor, enzyme, cell surface protein, a protein involved in apoptotic signaling, aromatase, helicase, mediator of a metabolic process (anabolism or catabolismantioxidant, protease, kinase, oxidoreductase, transferase, hydrolase, lyase, isomerase, ligase, enzyme regulator, signal transducer, structural molecule, binding activity (protein, lipid carbohydrate), cell motility protein, membrane fusion protein, cell communication mediator, regulator of biological processes, behavioral protein, cell adhesion protein, protein involved in cell death, protein involved in transport (including protein transporter activity, nuclear transport, ion transporter, channel transporter, carrier activity, permease, secretase or secretion mediator, electron transporter, chaperone regulator, nucleic acid binding, transcription regulator, extracellular organization and biogenesis regulator, and translation regulator). In certain embodiments, the POI is derived from a BET bromodomain-containing protein to which the protein binder is capable of binding or binds including, but not limited to, ASH1L, ATAD2, BAZ1A, BAZ1B, BAZ2A, BAZ2B, BRD1, BRD2, BRD3, BRD4, BRD5, BRD6, BRD7, BRD8, BRD9, BRD10, BRDT, BRPF1, BRPF3, BRWD3, CECR2, CREBBP, EP300, FALZ, GCN5L2, KIAA1240, LOC93349, MLL, PB1, PCAF, PHIP, PRKCBP1, SMARCA2, SMARCA4, SP100, SP110, SP140, TAF1, TAF1L, TIF1a, TRIM28, TRIM33, TRIM66, WDR9, ZMYND11, and MLL4.

In certain embodiments, a BET bromodomain-containing protein is BRD4.

As disclosed herein, the protein binder is a ligand which covalently or non-covalently binds to a POI which has been selected for proteasomal degradation by the PROTAC. The protein binder of the PROTAC may be any type of moiety capable of binding to the POI to be targeted for degradation via the ubiquitin-proteasome-system. In certain aspects, the protein binder is a polypeptide, a ligand, an aptamer, a nanoparticle, or a small molecule.

In certain embodiments, the protein binder is a human BET bromodomain targeting ligand identified in Filippakopoulos et al. (Nature 2010, 468, 1067-1073) titled “Selective Inhibition of Bet Bromodomains” such as JQ1; a ligand identified in Nicodeme et al. (Nature 2010, 468, 1119-1123) titled “Suppression of Inflammation by a Synthetic Histone Mimic”; Chung et al. (J. Med. Chem. 2011, 54, 3827-3838) titled “Discovery and Characterization of Small Molecule Inhibitors of the Bet Family Bromodomains”; a compound disclosed in Hewings et al. (J. Med. Chem. 2011, 54, 6761-6770) titled “3,5-Dimethylisoxazoles Act as Acetyl-Lysine-Mimetic Bromodomain Ligands”; a ligand identified in Dawson et al. (Nature 2011, 478, 529-533) titled “Inhibition of Bet Recruitment to Chromatin as an Effective Treatment for MLL-Fusion Leukaemia”; or a ligand identified in the following patent applications US 2015/0256700, US2015/0148342, WO 2015/074064, WO 2015/067770, WO 2015/022332, WO 2015/015318, and WO 2015/011084.

In certain versions, a linker is included in the PROTAC that covalently links the achiral CRBN ligand to the protein binder. In some embodiments, the linker can have a closed valence, and thus will contain one or more covalent bonds to ensure a complete valence, which may be to one or more hydrogen atoms, or in the case of carboxyl, sulfonyl, thiol, thiophenol, alcohol, or phenol groups can also be the deprotonated species and salts thereof, and for amines can also be the ammonium species and salts thereof. In one embodiment, the linker is a bivalent chemical group.

According to the disclosure, any desired linker can be used as long as the resulting compound is stable as part of a pharmaceutically acceptable dosage form, and itself is pharmaceutically acceptable.

In some embodiments, the linker has a chain of 2 to 14, 15, 16, 17, 18 or 20 or more carbon atoms of which one or more carbons can be replaced by a heteroatom such as O, N, S, or P. In certain embodiments the chain has 2, 3, 4, 5, 6, 7, 8, 9, 10, 11, 12, 13, 14, 15, 16, 17, 18, 19 or 20 contiguous atoms in the chain. For example, the chain may include 1 or more ethylene glycol units that can be contiguous, partially contiguous or non-contiguous (for example, 2, 3, 4, 5, 6, 7, 8, 9, 10, 11 or 12 ethylene glycol units). In certain embodiments the chain has at least 1, 2, 3, 4, 5, 6, 7, or 8 contiguous chains which can have branches which can be independently alkyl, heteroalkyl, aryl, heteroaryl, alkenyl, or alkynyl, aliphatic, heteroaliphatic, cycloalkyl or heterocyclic substituents.

In other embodiments, the linker can include or be comprised of one or more of ethylene glycol, propylene glycol, lactic acid and/or glycolic acid. In general, propylene glycol adds hydrophobicity, while propylene glycol adds hydrophilicity. Lactic acid segments tend to have a longer half-life than glycolic acid segments. Block and random lactic acid-co-glycolic acid moieties, as well as ethylene glycol and propylene glycol, are known in the art to be pharmaceutically acceptable and can be modified or arranged to obtain the desired half-life and hydrophilicity. In certain aspects, these units can be flanked or interspersed with other moieties, such as aliphatic, including alkyl, heteroaliphatic, aryl, heteroaryl, heterocyclic, cycloalkyl, etc., as desired to achieve the appropriate drug properties.

### Composition of Matter

The PROTAC as disclosed herein can be administered as the neat chemical, but are more typically administered as a pharmaceutical composition, that includes an effective amount for a host, typically a human, in need of such treatment for any of the disorders described herein. Accordingly, the disclosure provides pharmaceutical compositions comprising an effective amount of the PROTAC or pharmaceutically acceptable salt together with at least one pharmaceutically acceptable carrier for any of the uses described herein. The pharmaceutical composition may contain a PROTAC or salt as the only active agent, or, in an alternative embodiment, the PROTAC and at least one additional active agent.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the PROTAC is sufficient to provide a practical quantity of material for administration per unit dose of the PROTAC.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the PROTAC of the present disclosure.

The pharmaceutical compositions according to the present disclosure suitable for administration to an individual (e.g., suitable for human administration) are generally sterile and may further be free of detectable pyrogens or other contaminants contraindicated for administration to an individual according to a selected route of administration.

For oral preparations, the PROTAC can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The PROTAC can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### Method of Use

The PROTACs as disclosed herein can be used in an effective amount to treat a host, including a human, in need thereof, optionally in a pharmaceutically acceptable carrier to treat any of the disorders described herein.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Illustrative non-limiting disease states or conditions, including cancer, which may be treated using the PROTACs according to the present disclosure are set forth hereinabove.

The PROTACs and compositions as described herein can be used to degrade a POI which is a mediator of the disorder affecting the patient, such as a human. The control of protein level afforded by the PROTACs of the present disclosure provides treatment of a disease state or condition, which is modulated through the POI by lowering the level of that protein in the cell, e.g., cell of a patient. In certain embodiments, the method comprises administering an effective amount of the PROTAC as described herein, optionally including a pharmaceutically acceptable excipient, carrier, or adjuvant, i.e., a pharmaceutically acceptable composition, optionally in combination with another bioactive agent or combination of agents.

The term “disease state or condition” when used in connection with a PROTAC disclosed herein is meant to refer to any disease state or condition wherein protein dysregulation (i.e., the amount of protein expressed in a patient is elevated) occurs via an POI and where degradation of such protein in a patient may provide beneficial therapy or relief of symptoms to a patient in need thereof.

Nonlimiting examples of uses for the PROTACs are multiple myeloma, a hematological disorder such as myelodysplastic syndrome, cancer, tumor, abnormal cellular proliferation, HIV/AIDS, HBV, HCV, hepatitis, Crohn's disease, sarcoidosis, graft-versus-host disease, rheumatoid arthritis, Behcet's disease, tuberculosis, and myelofibrosis. Other examples include a myelo- or lymphoproliferative disorder such as B- or T-cell lymphomas, Waldenstrom's macroglobulinemia, Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, arthritis, and in particular rheumatoid arthritis, lupus, or type I diabetes; a disease of cardiologic malfunction, including hypercholesterolemia; an infectious disease, including viral and/or bacterial infection, as described generally herein; an inflammatory condition, including asthma, chronic peptic ulcers, tuberculosis, rheumatoid arthritis, periodontitis and ulcerative colitis.

An effective amount of the PROTAC (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the PROTAC or pharmaceutical composition.

### Examples

Glutarimides such as thalidomide, pomalidomide, and lenalidomide are the most frequently used ligands to recruit E3 ubiquitin ligase cereblon (CRBN) for the design of PROTACs. Due to the rapid and spontaneous racemization of glutarimides, most CRBN-recruiting PROTACs are synthesized as racemates. Since the (S)-enantiomer is primarily responsible for binding to CRBN, the existence of the largely inactive (R)-enantiomer complicates many aspects of the drug development process. In this Example, we demonstrated that substituted achiral phenyl dihydrouracils can be used as a novel class of CRBN ligands for the development of PROTACs. Although the parent PDHU 2 has minimal binding affinity to CRBN, we found that some substituted PDHUs had comparable binding affinity to lenalidomide. The PDHUs also have greater stability than the lenalidomide. Most importantly, potent non-racemic BRD4 degraders could be developed for the first time by employing achiral 1,2,3-trisubstituted PDHUs.

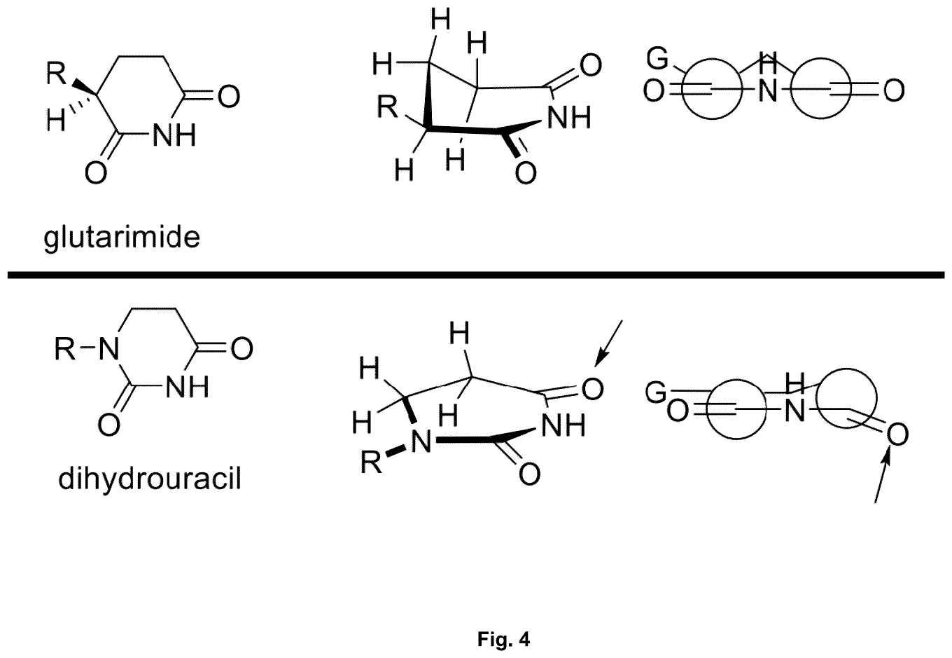

We began our investigation by examining the conformations of simple glutarimide and dihydrouracil by extracting the relevant X-ray structures 4CI1,³² 1R2Z⁴², respectively, from Protein Data Bank. Glutarimide has a half-chair conformation, where five atoms in the imide ring are in the same plane. The remaining methylene group in the ring and the “R” substituent are out of the plane. In contrast, dihydrouracil is much flatter. The R-substituent on the nitrogen is in the same plane as the imide, while the carbonyl group pointed by the arrow in FIG. 4 and its adjacent methylene group are twisted out of the plane slightly. Although the different orientation of R group and the twisted imide group significantly lowered the binding affinity of simple dihydrouracil (e.g., R=Ph) with the CRBN protein complex, structure-based analysis and judicious introduction of selected substituents recovered the binding affinity as shown in the results below. Most importantly, the achiral dihydrouracils can be used as ligands for the development of potent PROTACs.

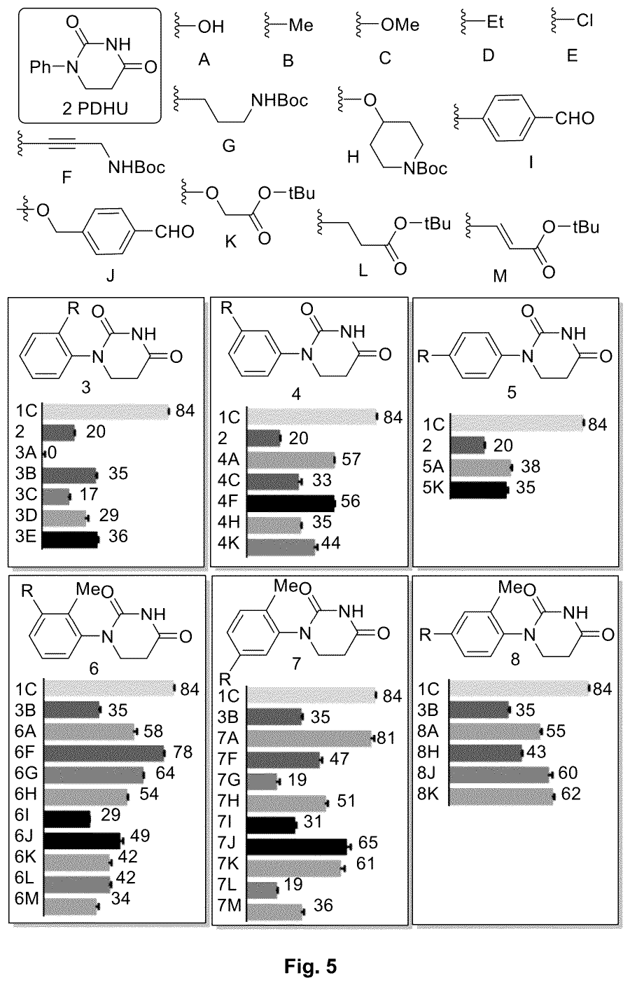

We first prepared simple phenyl dihydrouracil (PDHU) 2 (FIG. 5) and tested its binding with the CRBN-DDB1 complex using a known fluorescence polarization (FP) assay.³² Known compound lenalidomide 1C was used as the positive control. The conditions were adjusted to allow around 84% of the maximum signal for 1 μM of lenalidomide. All analogs were tested at a single concentration of 1 μM three times. As shown in FIG. 5, the binding affinity observed between 2 and CRBN-DDB1 complex was only around 20%, which is not surprising based on the conformational analysis as discussed above. To improve the binding affinity, a series of disubstituted PDHUs were designed and synthesized. Various substituents were installed at the ortho, meta, and para positions of the phenyl group in PDHUs and compared with the parent compound 2 for relative binding affinity. Interestingly, analogue 3A with an ortho hydroxyl substituent almost completely lost the binding affinity, while analogues 4A and 5A with a meta and para OH enhanced the binding to 57% and 38%, respectively. Methyl, ethyl and chloro substituents on the ortho-position (3B, 3D, 3E) improved the binding to 35%, 29% and 36% from the original 20% of the parent compound 2. Compound 3C with a methoxy substituent on the ortho-position decreased the binding, while placing the MeO on the meta-position (4C) improved the binding to 33%. Compounds 4F and 4K bearing longer substituents on the meta-position further improved the binding to 56% and 44%, respectively. Substituents on the para-positions were also tolerated, though they did not improve the binding as much as those on the meta-positions as shown in 5A and 5K.

Based on the structure-activity relationship (SAR) of the disubstituted PDHUs, we designed the second series of trisubstituted PDHUs to further enhance the binding affinity. In consideration of binding results, ligand efficiency, metabolic stability and magic methyl effect, +3.44 ortho methyl was selected to explore its synergetic effect of binding improvement with the third substituents. Various 1,2,3-, 1,2,5- and 1,2,4-trisubstituted PDHUs were designed, synthesized, and compared with the positive control lenalidomide and compound 3B with an ortho methyl substituent. We prepared more compounds with meta-substituents on the phenyl group of PDHUs since our results from the disubstituted PDHUs indicated that meta-substituents improved the binding more than the para-substituents. Among different groups, substituent A and F had the strongest synergetic effect. For example, the binding of 6F and 7A were improved to 78% and 81%, respectively. Most other trisubstituted PDHUs also showed better binding affinity than 3B.

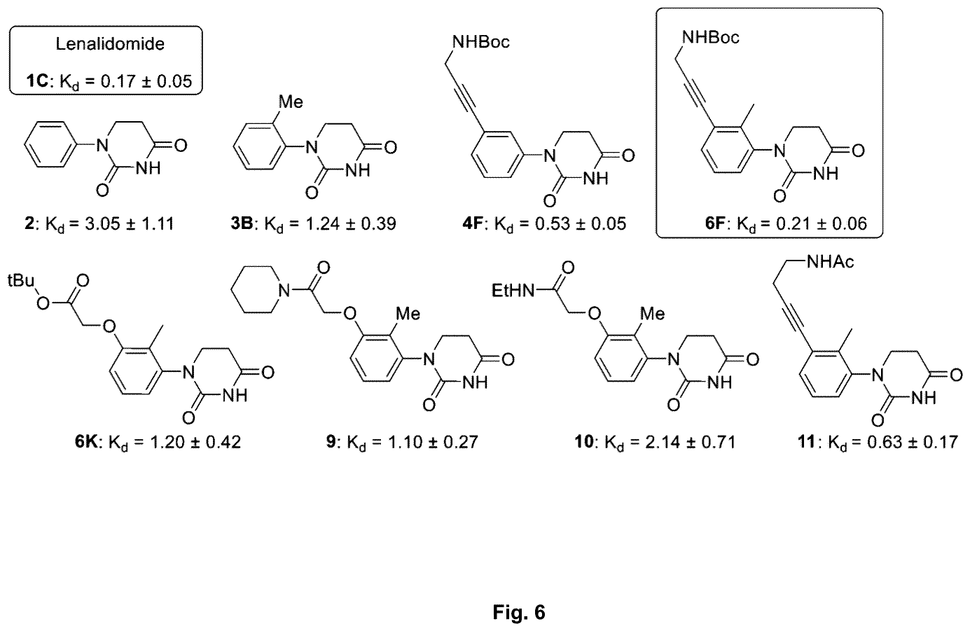

It is easiest to install building blocks F and K (FIG. 5) to the phenyl group on PDHUs. The tBu group in these two building blocks can be replaced by various linkers for the development of non-racemic PROTACs that can recruit CRBN. With these in mind, we next measured the K_{d}s for lenalidomide 1C, parent compound PDHU 2, mono-substituted PDHU 3B, disubstituted PDHU 4F, and trisubstituted PDUs 6F and 6K for further comparison (FIG. 6). The K_{d} (0.17 μM) we observed in the FP assay for lenalidomide 1C is very close to what was reported in the literature (K_{d}=0.18 μM).³² It is worth noting that parent PDHU 2 is 18 times weaker than lenalidomide 1C. The installation of an ortho-Me in 3B increased the affinity about 2.5 times. The 1,2,3-trisubstituted PDHU 6F has a comparable K_{d} to lenalidomide. The difference between 6F and 4F confirms that the ortho- and meta-substituents in 6F both contributed to the binding. The meta-substituent in PDHU 6K does not further increase the binding compared to 3B. We then prepared compounds 9A and 9B by replacing the tBu with an amide functional group to confirm this observation. Similar K_{d}s were observed for compounds 6K, 9 and 10. We also extended the propargyl amide linker in 6F to homopropargyl amide in 11. The affinity dropped three fold in this case.

We then further evaluated the stability of selected compounds (6F and 9-11) in human liver microsomes (hLM), human plasma (hP) and phosphate buffer with different pHs (Table 1). These compounds showed comparable stability to lenalidomide in hLM. In terms of compound stability in hP, after 4 h of incubation, only 62% of lenalidomide remained. By contrast, 97% of 6F and over 80% of 9-11 remained. Under acidic conditions, all compounds are very stable. However, in PB7.4 buffer, only 39% of lenalidomide was remained after 24 h of incubation at 37° C., while hydrolysis of 1,2,3-trisubstituted PDHUs 6F and 9-11 was not detected. PDHUs 6F and 9-11 are also much more stable than lenalidomide at higher pH such as PB8.8. Other than racemization, the ring opening of glutarimides by hydrolysis is another stability issue of CRBN-recruiting PROTACs.⁴⁵ Our results showed that the dihydrouracil motif has much higher stability and is less prone to hydrolysis. Based on the above data, we can conclude that the achiral CRBN ligands with 1,2,3-trisubstituted PDHUs such as 6F can have similar binding affinity and much better stability profiles compared with lenalidomide.

TABLE 1
Stability of lenalidomide and two 1,2,3-trisubstituted PDHUs

| Compound | hLM^{[a]} | hP^{[b]} | PB1.0^{[c]} | PB7.4^{[d]} | PB8.8^{[e]} |
|---|---|---|---|---|---|
| Lenalidomide | 89 | 62 | >99 | 39 | 0^{[f]}, 20^{[g]} |
| 6F | 87 | 97 | >99 | >99 | 93 |
| 9 | 85 | 81 | >99 | >99 | 95 |
| 10 | 100 | 88 | >99 | >99 | 95 |
| 11 | 90 | 102 | >99 | >99 | 90 |

^{[a]}Percent compound remaining after 0.5 h.
^{[b]}Percent compound remaining after 4.0 h.
^{[c-f]}Percent compound remaining after 24 h.
^{[g]}Percent compound remaining after 2 h.

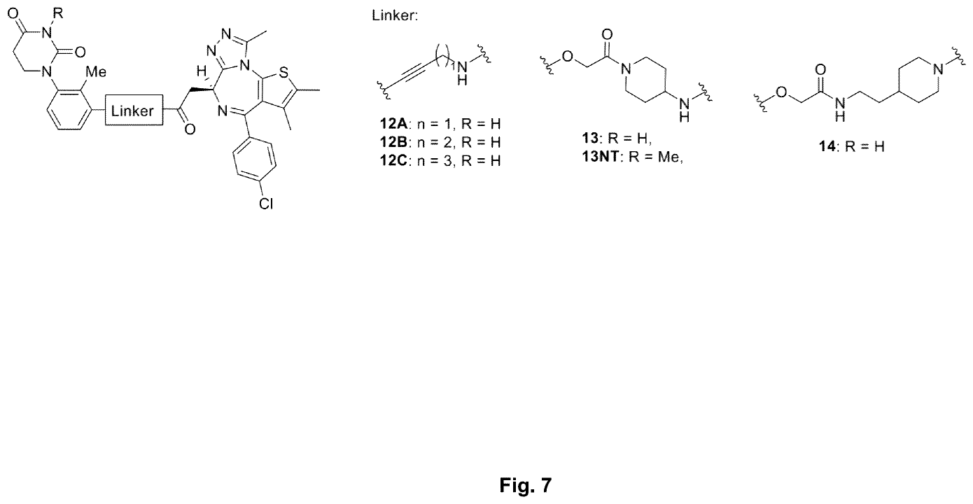

The discovery of the achiral CRBN ligands provides an opportunity to develop various non-racemic PROTACs that can recruit CRBN. To demonstrate the utility of these new CRBN ligands, (e.g., 6F and 9A/B), we attached JQ1, a well-known ligand for BRD4, to the 1,2,3-trisubstituted PDHU and prepared potential BRD4 degraders 12A-12C, 13 and 14 bearing several different linkers (FIG. 7).

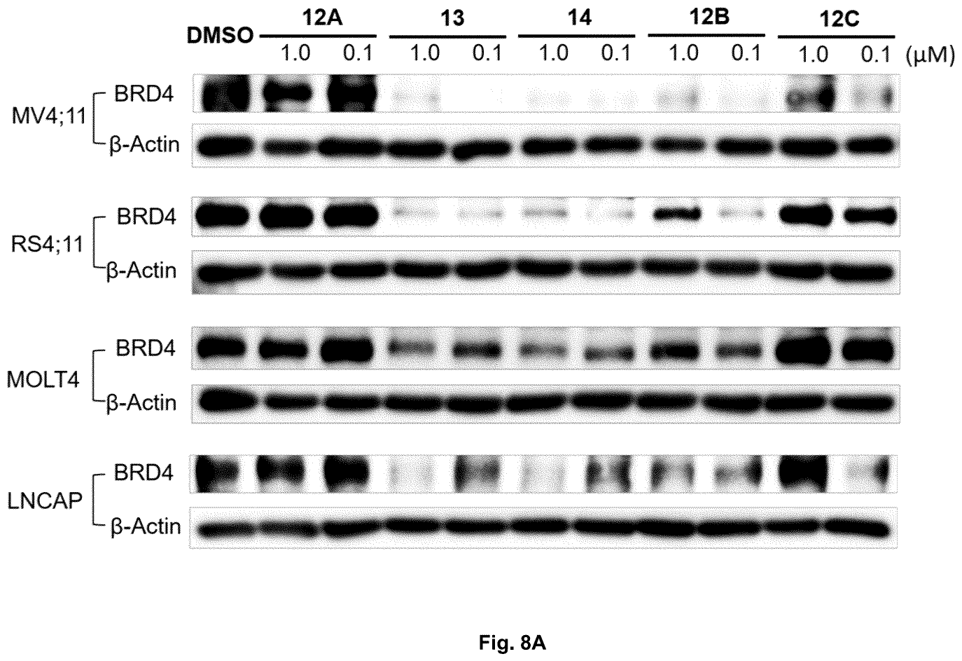

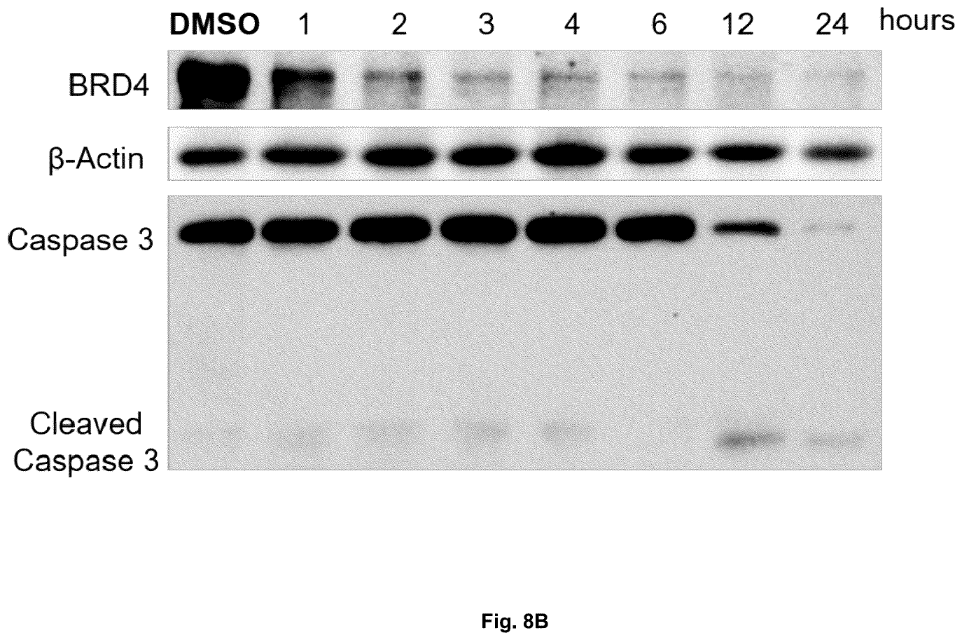

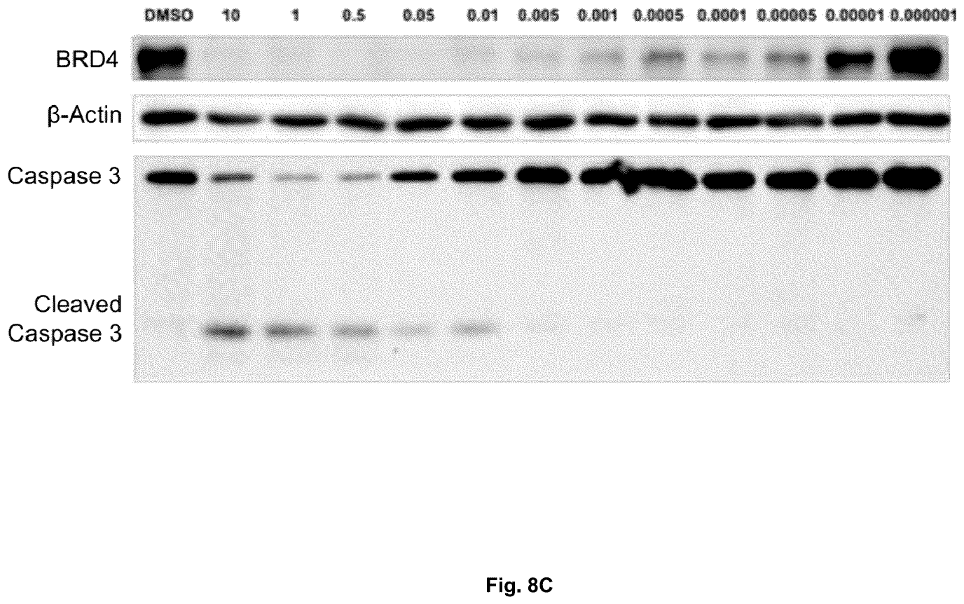

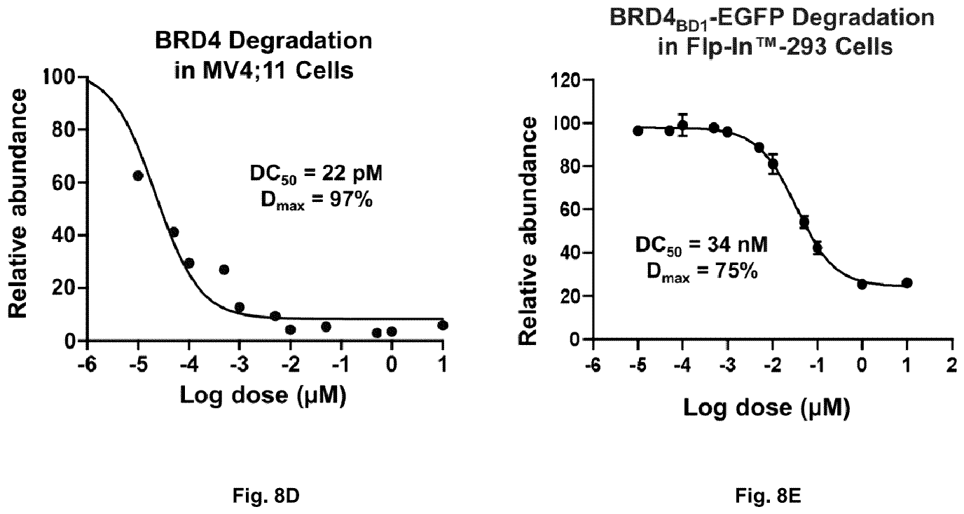

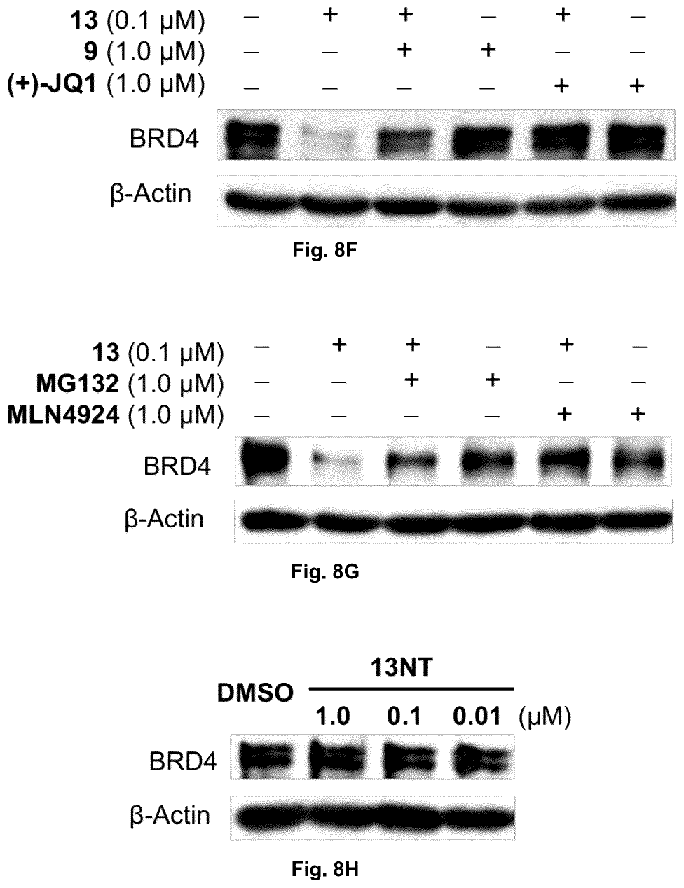

These compounds were then tested in four different cell lines at two different concentrations (1.0 and 0.1 μM) for the degradation of BRD4. Western blot analysis indicates that all compounds could induce the degradation of BRD4 protein in all four cell lines (FIG. 8A). The weaker activity observed for compounds 12A and 12C are likely due to inappropriate linker between the two ligands. While compounds 12B, 13 and 14 displayed the most activity, compound 13 appears to be slightly more potent. Our results indicate that the degradation efficiency is related to multiple parameters such as the length and type of linkers, in addition to the binding affinity of the achiral ligand to CRBN. Compound 13 was then selected for further characterization. We first studied the time course and dose response of degrader 13 (FIGS. 8B-8E). Degradation of BRD4 occurred as early as 1 h post-treatment (FIG. 8B). Degrader 13 also induced significant degradation of BRD4 at pM concentrations in MV4; 11 cells with a DC₅₀ of 22 pM and Dₘₐₓ of 97% (FIGS. 8C and 8D). We also verified the degradation effect of 13 in a Flp-In™-293 cell line that stably expresses e-GFP-tagged BRD4 bromodomain (BD1) fusion and a non-targeted mCherry endogenous control (FIG. 8E).⁴⁶ In the Flp-In™-293 cells, a DC₅₀ of 34 nM and a Dₘₐₓ of 75% at 10 μM was observed. Interestingly, no obvious “Hook effect”47 was observed with up to 10 μM of degrader 13. The degrader was successful in inducing caspase 3 cleavage, a biomarker of cell apoptosis, at 10 nm to 10 UM concentrations.

We next verified the mechanism of action of degrader 13 (FIGS. 8G-8H). Pretreatment of the cells with POI ligand JQ1, achiral CRBN ligand 9, proteasome inhibitor MG132, and neddylation inhibitor MLN492448 abolished the BRD4 degradation induced by 13, suggesting that the degradation involves the engagement of BRD4, CRBN, proteasome, and Cullin-RING E3 ligase complex. We also prepared negative control 13NT (FIG. 7), which has an additional methyl group on the imide motif that prevents the binding to CRBN.³² No degradation activity of BRD4 was observed for 13NT in MV4; 11 cells at several different concentrations, further confirming the involvement of CRBN (FIG. 8H).

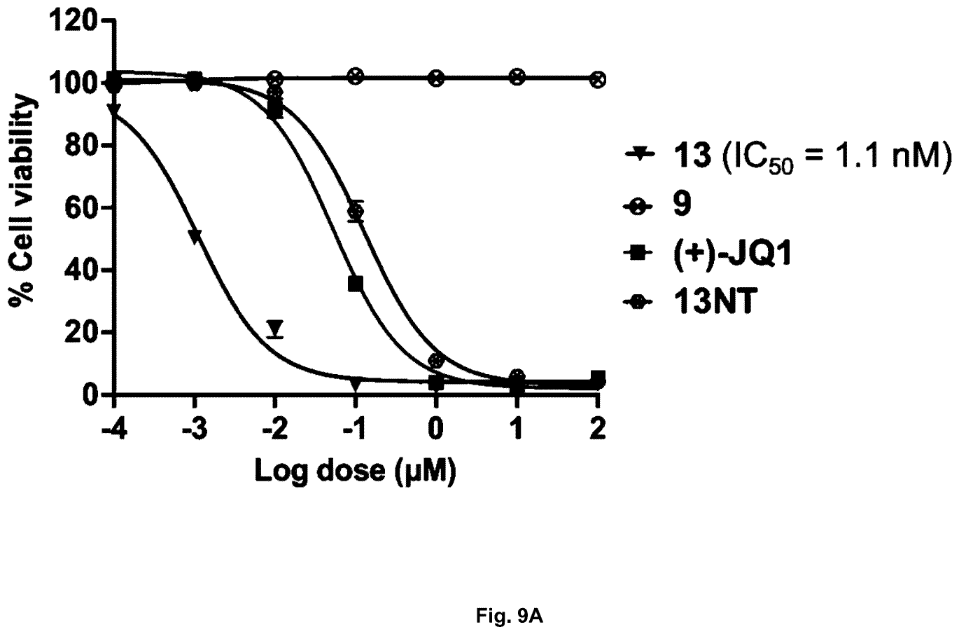

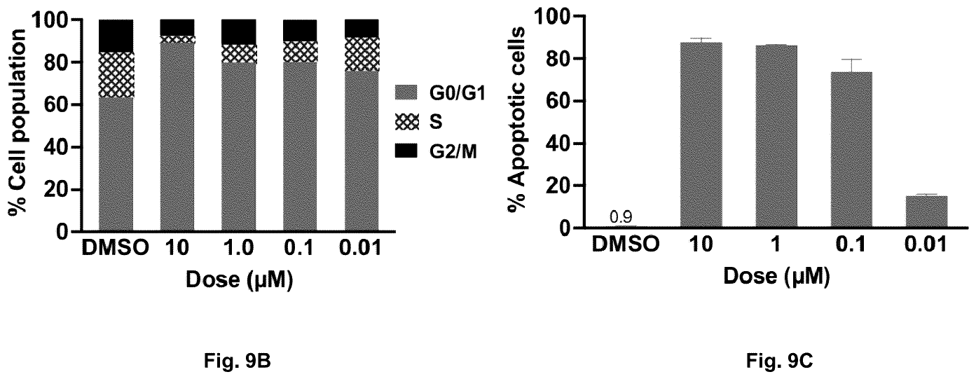

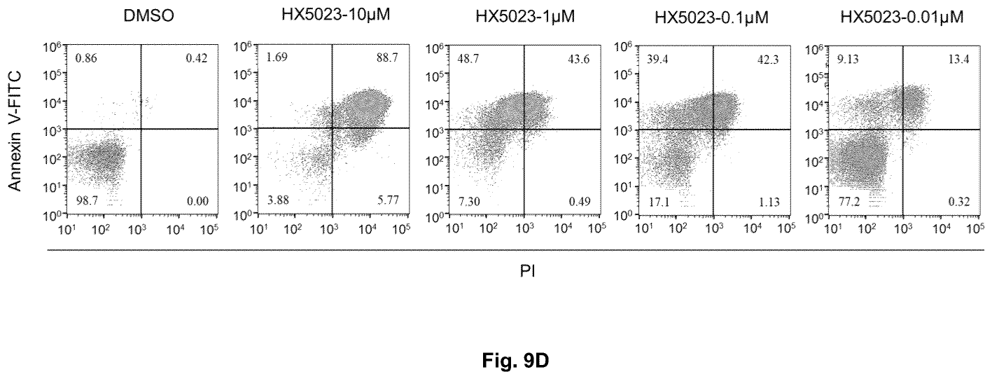

We next investigated the functional outcomes of degraders 13 bearing achiral CRBN ligands including cell proliferation, cell cycle arrest, and cell apoptosis (FIGS. 9A-9D). First, MV4; 11 cells were treated by BRD4 degraders 13, POI ligand (+)-JQ1, CRBN ligand 9, and compound 13NT for 72 h. Compound 13 showed the most potent anti-proliferation activity with an IC₅₀ value around 1.1 nM (FIG. 9A). POI ligand (+)-JQ1 showed weaker anti-proliferation activity than 13, but higher activity than compound 13NT, which has a very similar structure to 13 but without CRBN recruiting ability. Compound 9, which has the entire CRBN ligand and most parts of the linker, did not show any anti-proliferation activity at up to 100 μM. All these trends are consistent with their mechanism of action. BRD4 degraders 13 can also induce G1 phase arrest, decrease the cell population in S phase, (FIG. 9B) and induce cell apoptosis in a dose-dependent manner (FIGS. 9C and 9D) in MV4; 11 cells.

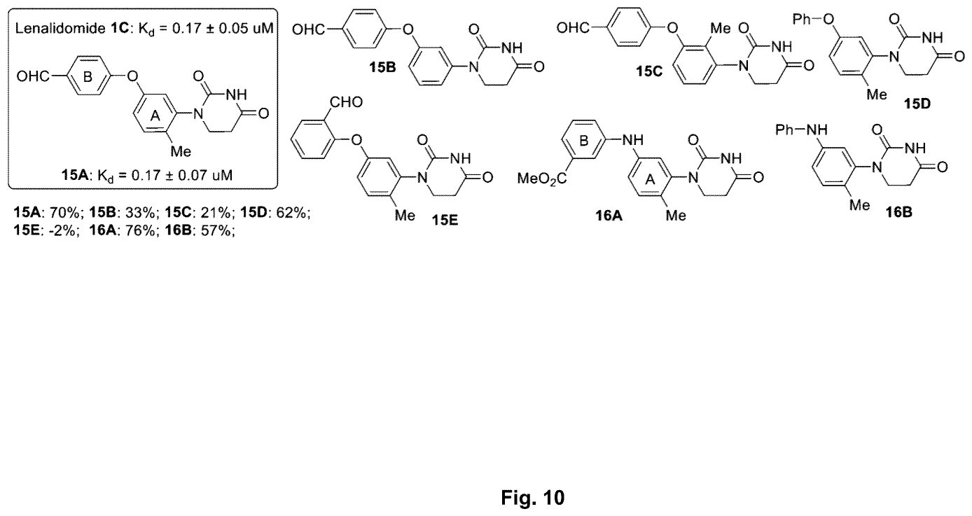

In addition to compound 6F with a K_{d} of 0.21 μM, we also identified another potent lead compound 15A with a K_{d} of 0.17 μM (FIG. 10). We also prepared several analogues with the diphenyl ether functionality (e.g., compounds 15B-E) and compared their binding affinity at single concentration under similar conditions used as FIG. 5. It is interesting to note that the presence and position of the methyl substituent on ring-A is essential (70% for 15A vs 33% for 15B and 21% for 15C). The electron-withdrawing formyl group on ring-B also contributes to the affinity as 15D is slightly weaker than 15A. The ortho-substituted formyl group in compound 15E completely abolishes the binding. We also prepared two phenyl aniline derivatives 16A and 16B. Both of them remain relatively high binding affinity and the meta-substituted methylcarboxylate in ring-B is beneficial.

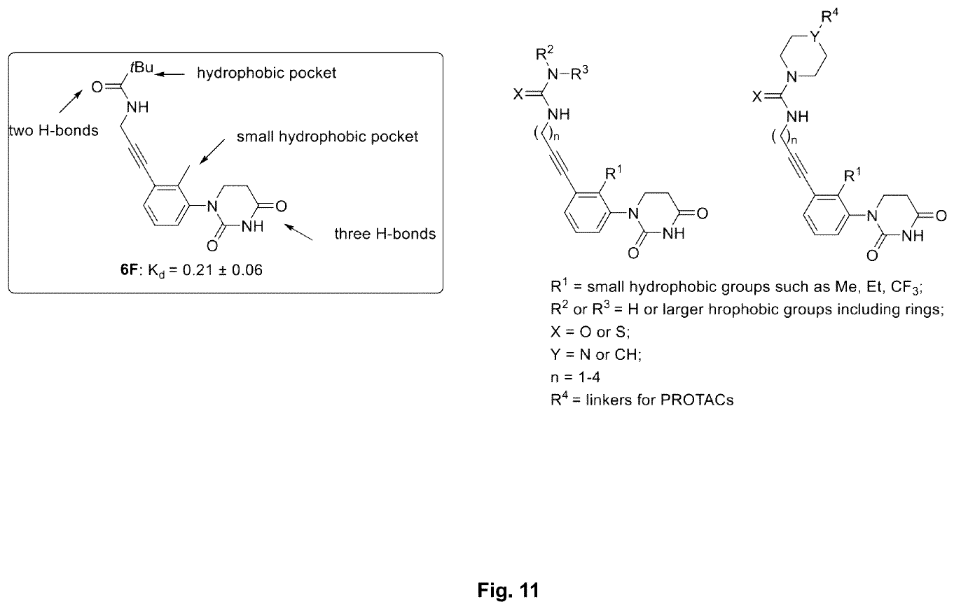

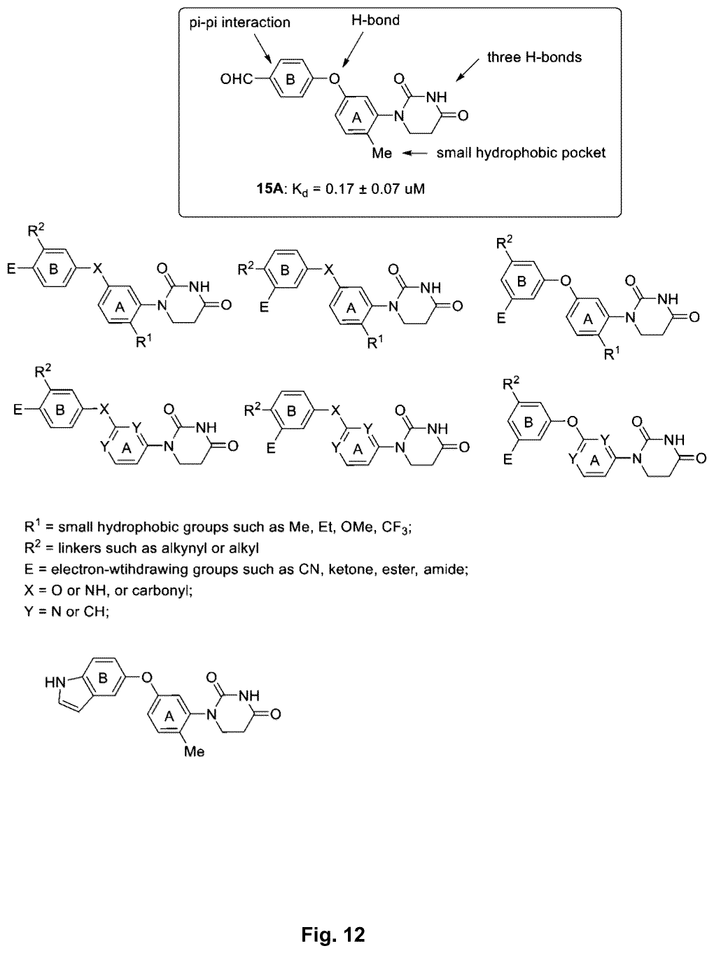

Based on our current experimental data for compounds 6F and 15A, and modeling studies, compounds as shown in FIGS. 11 and 12 will further be prepared and tested.

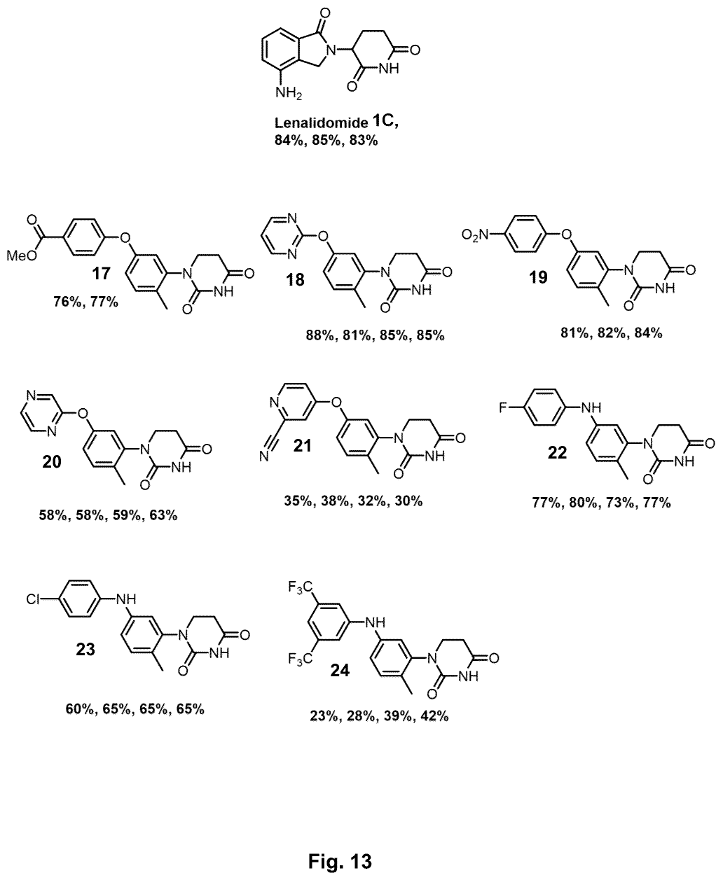

Compounds 17, 18, 19, 20, 21, 22, 23, and 24 were tested for binding with the CRBN-DDB1 complex as described above for FIG. 5. The compounds and their relative binding affinities are shown in FIG. 13.

### Materials and Methods:

Achiral CRBN ligands and BRD4 PROTACs were synthesized as shown in scheme 1. Various substituted PDHUs S2 can be prepared from the corresponding anilines S1 by adapting literature procedures through conjugate addition followed by cyclization (eq 1).³⁸ More substituents can be introduced to the phenyl group of PDHUs by Sonogashira coupling (eq 2), alkylation (eq 3 and eq 6), Suzuki cross coupling (eq 4), or Heck cross-coupling (eq 5). Further manipulations such as reduction (eq 7) and de-protection of the Boc group followed by amide formation can afford ligands 6G, 9, and 11, respectively.

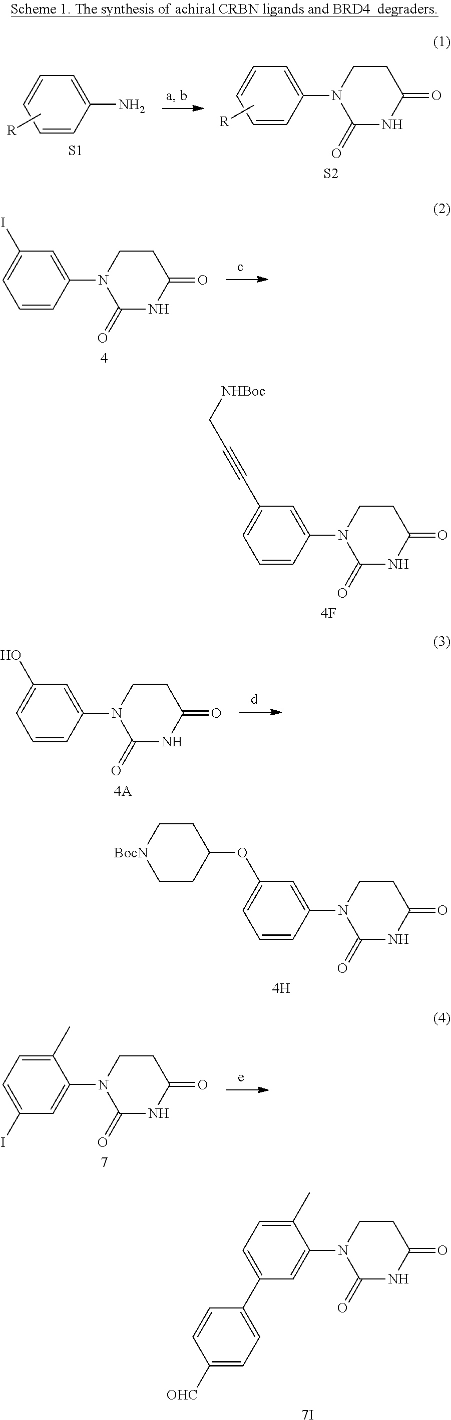

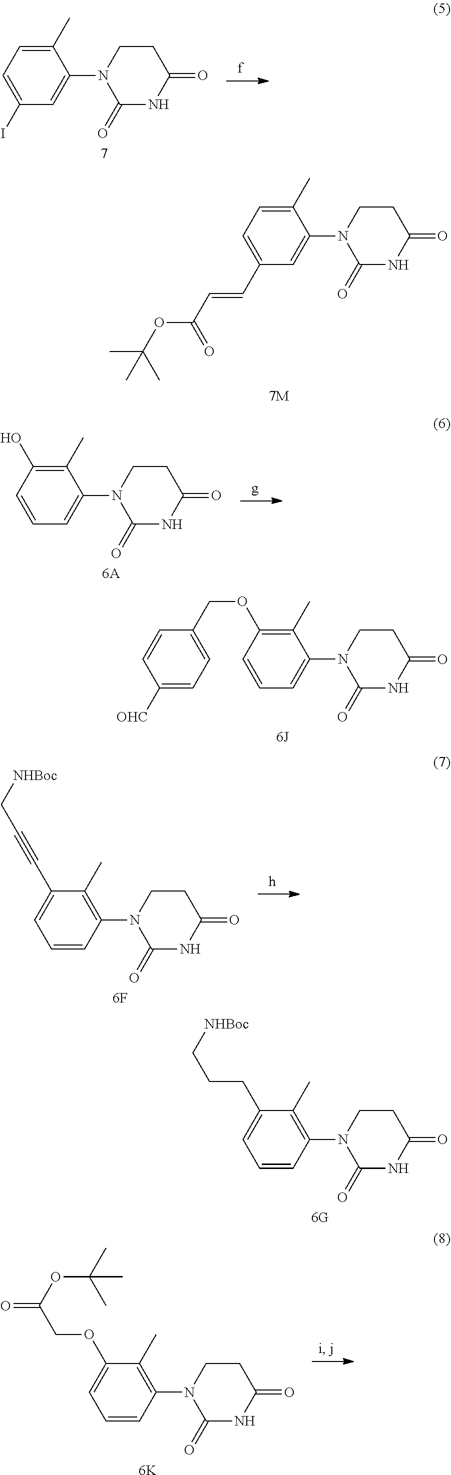

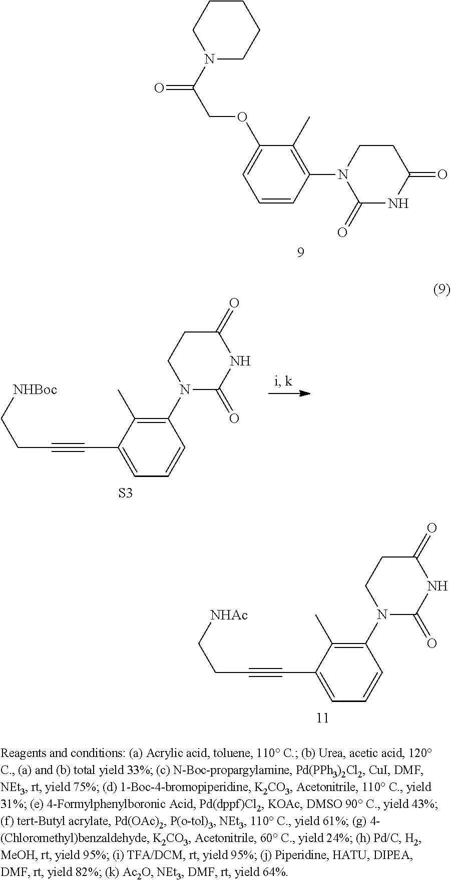

BRD4 PROTACs bearing achiral CRBN ligands were synthesized as shown in scheme 2. Removal of the Boc protecting group in intermediate S4 followed by amide coupling with JQ1 afforded PROTACs 12A-C. Removal of the tBu protecting group in intermediate S5 followed by coupling with a linker, de-protection, and reaction with JQ1 yielded PROTACs 13, 14 and the negative control 13NT.

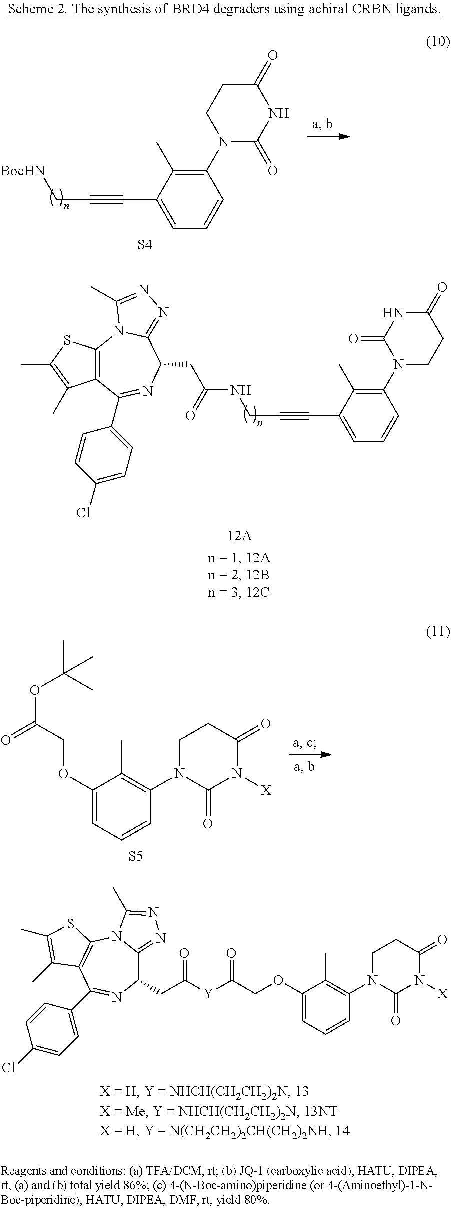

Cell Culture Three human leukemia cell lines (MV4; 11, RS4; 11, MOLT4) and one prostate cancer cell line (LNCAP) were obtained from ATCC, expanded, and frozen down in cryogenic vials. Cells were thawed and used within 20 passages in the experiments. All cells were cultured in RPMI1640 media (Corning) supplemented with 10% fetal bovine serum (FBS) and 1% Penicillin and Streptomycin in a CO₂ incubator (37° C., with humidified 5% CO₂ atmosphere).

AlamarBlue Cell Viability Cells were seeded in a 96-well plate at a density of 5000-10000 cells in 100 μl media per well and treated with DMSO control or test compounds at indicated doses. After incubated in CO₂ incubator for 72 hours, cells were treated with 10 μL AlamarBlue solution (440 mM)). Fluorescence was measured in a plate reader with an excitation wavelength at 530-560 nm and emission wavelength at 590 nm. The fluorescence was normalized to the DMSO-treated cells, and the IC₅₀ was calculated using GraphPad Prism 9 software.

Western Blot Assay Cells were treated with test compounds at indicated doses, incubated for the indicated time, and then lysed in RIPA plus protease inhibitors. The protein concentration was determined by the BCA assay. Equivalent amounts of protein were loaded and separated in 7.5% SDS-PAGE gels and transferred to PVDF membranes. Membranes were blocked in 5% BSA/TBST solution and then incubated with the appropriate primary antibodies diluted in 5% BSA/TBST in a cold room overnight. After being washed, the membranes were incubated with the appropriate HRP-conjugated secondary antibodies in 2% BSA/TBST for one h at room temperature and then washed again. Bound antibodies were visualized using ECL assay (Bio-Rad), and images were captured using the ChemidocTMMP imaging system (Bio-Rad). All antibodies were purchased from Cell signaling Technology, including Anti-Brd4 (CS #13440), anti-Caspase 3 (CS #9662), Anti-β-Actin (CS #3700), and HRP-conjugated anti-mouse IgG (CS #7076) and HRP-conjugated anti-rabbit IgG (CS #7074).

Flow Cytometry Cell apoptosis and cell cycle were detected by Attune Flow cytometer (Thermo Fisher). Cells were seeded in a 6-well plate and treated with the indicated concentrations of compounds for 48 h. For samples for cell apoptosis detection, cells were harvested and incubated in Annexin V-FITC and PI reagents each for 15 minutes (Annexin V-FITC apoptosis detection kit, Invitrogen). For the samples for cell cycle detection, cells were fixed in 70% cold ethanol overnight and stained with PI (500 μg/ml) for 15 minutes. Flow data were analyzed using Flowjo software.

Cellular Degradation in Flp-In™-293 Cell BRD4BD1 mammalian pcDNA5/FRT Vector (Ampicillin and Hygromycin B resistant) with MCS-eGFP-P2A-mCherry was obtained from Fischer's lab.³⁵ Stable cell lines expressing eGFP-protein fusion and mCherry reporter were generated using the Flp-In 293 system according to the manual from the vendor (Thermo Fisher). Plasmid (0.4 μg) and pOG44 (3.6 μg) DNA were pre-incubated in Opti-MEM media with 12 μL FuGene HD and added to Flp-In 293 cells (REF R75007, ThermoFisher) containing DMEM media per well in a 6-well plate format. Cells were propagated after 48 h and transferred into a 10 cm² plate in DMEM media containing 50 μg/ml of Hygromycin B as a selection marker. Positive colonies were split into a 60 mm dish until confluent and subsequently propagated. eGFP and mCherry signal were confirmed by flow cytometry for assay validation.

Cells were seeded at 5×10\hat{ }5 per well in 24-well plates one day prior to compound treatment. Titrated compounds were incubated with cells for 24 h followed by detachment and resuspension in PBS with 2% FBS, filtration, and transferred into 5 mL polystyrene tubes for analysis by flow cytometry (Attune, Thermo Fisher). Signal from at least 50,000 events per well was acquired, and the eGFP and mCherry fluorescence monitored. Data were analyzed using FlowJo (FlowJo, LCC). Forward and side scatter outliers were removed by gating. The eGFP signal abundance relative to mCherry was then quantified as a ten-fold amplified ratio using the formula: 10×eGFP/mCherry. The median fluorescence intensity (MFI) was calculated for eGFP and mCherry for each sample and normalized to the DMSO ratio.

General Information on Synthetic Chemistry All reactions were conducted under a positive pressure of dry argon in glassware that had been oven-dried prior to use. Anhydrous solutions of reaction mixtures were transferred via an oven-dried syringe or cannula. All solvents were dried prior to use unless noted otherwise. Thin-layer chromatography (TLC) was performed using precoated silica gel plates. Flash column chromatography was performed with the silica gel. 1H and 13C nuclear magnetic resonance (NMR) spectra were recorded on Bruker 400, 500, 600 MHz and Varian 500 MHz spectrometers. ¹H NMR spectra were reported in parts per million (ppm) referenced to 7.26 ppm of CDCl₃ or referenced to the center line of a septet at 2.50 ppm of DMSO-d₆. Signal splitting patterns were described as singlet (s), doublet (d), triplet (t), quartet (q), quintet (quint), or multiplet (m), with coupling constants (J) in hertz. High-resolution mass spectra (HRMS) were performed on an electron spray injection (ESI) TOF mass spectrometer. The liquid chromatography-mass spectrometry (LC-MS) analysis of final products was processed on an Agilent 1290 Infinity II LC system using a Poroshell 120 EC-C18 column (5 cm×2.1 mm, 1.9 μm) for chromatographic separation. Purity is >95% as determined by HPLC for all final compounds tested for biological activities.

### General Procedure for Preparation of Phenyl Dihydrouracils

In a 250 mL flask with a magnetic stirring bar, a solution of known 4-hydroxyaniline (4.37 g, 40.0 mmol) in toluene (50 mL, 0.8 M) was added acrylic acid (3.57 mL, 52.0 mmol). The reaction mixture stirred at 110° C. until the staring material disappeared as indicated by TLC, then the toluene was removed by rotavapor. Acetic acid (60 mL) and urea (7.20 g, 120.0 mmol) was then added to the flask and heated to 120° C. for 16 hours. Most of the acetic acid was removed by rotavapor. The residue was then dissolved in water (100 mL) and extracted by ethyl acetate (3×100 mL). The organic layers were combined, dried over Na₂SO₄, and concentrated under reduced pressure. The solid residue was suspended in ethyl acetate (10 mL) and stirred for 2 hours. The slurry was filtrated and the solid was wash by ethyl acetate (2×5 mL). Compound 5A (2.75 g, yield 33%) was obtain as solid.

### General Procedure for Preparation of 4F, 6F and 7F by Sonogashira Couplings

A 25 mL flask was charge with magnetic stirring bar, compound 4 (316 mg, 1.0 mmol), N-Boc-propargylamine (466 mg, 3.0 mmol), Pd(PPh₃)₂Cl₂ (35 mg, 0.05 mmol) and CuI (10 mg, 0.05 mmol), evacuated and backfilled with argon. Dimethylformamide (DMF) (2.5 mL) and Tricthylamine (NEt₃) (2.5 mL) were successively added by syringe. The reaction mixture stirred at room temperature until the staring material compound 4 disappeared as indicated by TLC. The mixture was partitioned between ethyl acetate and saturated solution of sodium bicarbonate, the organic layer was washed with brine, dried over Na₂SO₄, and concentrated in vacuum. The residue was purified by column chromatography on silica to provide 4F (260 mg, yield 75%) as white solid.

### General Procedure for Preparation of 4H, 6H, 7H and 8H

A 35 mL pressure tube was charge with magnetic stirring bar, compound 4A (412 mg, 2.0 mmol). Next 1-Boc-4-bromopiperidine (1585 mg, 6.0 mmol), K₂CO₃ (910 mg, 6.6 mmol) and acetonitrile (10 mL) was added. Then the reaction mixture was stirred at 110° C. for 18 hours. The mixture was partitioned between ethyl acetate and water, the organic layer was washed with brine, dried over Na₂SO₄, and concentrated in vacuum. The residue was purified by column chromatography on silica to provide 4H (241 mg, yield 31%) as white solid.

### General Procedure for Preparation of 61 and 71 by Suzuki Couplings

A 25 mL flask was charge with magnetic stirring bar, compound 7 (330 mg, 1.0 mmol), 4-Formylphenylboronic acid (300 mg, 2.0 mmol), Pd(dppf)Cl₂ (36 mg, 0.05 mmol) and KOAc (295 mg, 3.0 mmol), evacuated and backfilled with argon. Dimethyl sulfoxide (DMSO) (5.0 mL) was added by syringe. The reaction mixture stirred at 90° C. until the staring material compound 7 disappeared as indicated by TLC. The mixture was partitioned between ethyl acetate and saturated solution of sodium bicarbonate, the organic layer was washed with brine, dried over Na₂SO₄, and concentrated in vacuum. The residue was purified by column chromatography on silica to provide 71 (133 mg, yield 43%) as white solid.

### General Procedure for Preparation of 6J, 7J and 8J

A 35 mL pressure tube was charge with magnetic stirring bar, compound 6A (440 mg, 2.0 mmol). Next 4-(Chloromethyl)benzaldehyde (310 mg, 2.0 mmol), K₂CO₃ (608 mg, 4.4 mmol) and acetonitrile (10 mL) was added. Then the reaction mixture was stirred at 80° C. until the staring material compound 7 disappeared as indicated by TLC. The mixture was partitioned between ethyl acetate and water, the organic layer was washed with brine, dried over Na₂SO₄, and concentrated in vacuum. The residue was purified by column chromatography on silica to provide 6J (162 mg, yield 24%) as white solid.

### General Procedure for Preparation of 4K, 5K, 6K, 7K and 8K

A 35 mL pressure tube was charge with magnetic stirring bar, compound 4A (620 mg, 3.0 mmol). Next tert-Butyl bromoacetate (0.465 mL, 3.15 mmol), K₂CO₃ (910 mg, 6.6 mmol) and acetonitrile (15 mL) was added. Then the reaction mixture was stirred at 80° C. until the staring material compound 7 disappeared as indicated by TLC. The mixture was partitioned between ethyl acetate and water, the organic layer was washed with brine, dried over Na₂SO₄, and concentrated in vacuum. The residue was purified by column chromatography on silica to provide 4K (662 mg, yield 69%) as white solid.

### General Procedure for Preparation of 6M and 7M by Heck Reaction

A 25 mL flask was charge with magnetic stirring bar, compound 7 (165 mg, 0.5 mmol), Pd(OAc)₂ (11 mg, 0.05 mmol) and P(o-tol)₃ (38 mg, 0.12 mmol), evacuated and backfilled with argon. Dimethylformamide (DMF) (3.0 mL), Triethylamine (NEt₃) (0.21 mL, 1.5 mmol) and tert-Butyl acrylate (0.22 mL, 1.5 mmol) were successively added by syringe. The reaction mixture stirred at 100° C. until the staring material compound 7 disappeared as indicated by TLC. The mixture was partitioned between ethyl acetate and saturated solution of sodium bicarbonate, the organic layer was washed with brine, dried over Na₂SO₄, and concentrated in vacuum. The residue was purified by column chromatography on silica to provide 7M (101 mg, yield 61%) as white solid.

### General Procedure for Preparation of 6G, 7G, 6L and 7L

A 20 mL vial was charge with magnetic stirring bar and compound 6F (50 mg, 0.14 mmol). Next methanol (3.0 mL) and palladium on carbon (10 wt. %) (20 mg) was added. A hydrogen balloon was connected with the reaction mixture through a needle. The reaction mixture stirred at room temperature until the staring material compound 6F disappeared as indicated by TLC. The mixture was filtrated by celite and wash by methanol (3×5 mL). The methanol solution was concentrated in vacuum to provide 6G (48 mg, yield 95%) as white solid.

### General Procedure for Preparation of 9 and 10.

A 20 mL vial was charge with magnetic stirring bar and compound 6K (67 mg, 0.2 mmol).

Next dichloromethane (DCM) (0.5 mL) and trifluoroacetic acid (TFA) (0.75 mL) was added. The reaction mixture stirred at room temperature until the staring material compound 6K disappeared as indicated by TLC. The mixture was concentrated in vacuum to provide white solid. In the same vial, DMF (1.0 mL), piperidine (0.02 mL, 0.2 mmol), triethylamine (0.112 mL, 0.8 mmol) and 1-[Bis(dimethylamino)methylene]-1H-1,2,3-triazolo[4,5-b]pyridinium 3-oxide hexafluorophosphate (HATU) (91 mg, 0.24 mmol) were successively added. The reaction mixture stirred at room temperature until the staring material disappeared as indicated by TLC. The mixture was partitioned between ethyl acetate and saturated solution of sodium bicarbonate, the organic layer was washed with brine, dried over Na₂SO₄, and concentrated in vacuum. The residue was purified by column chromatography on silica to provide 9 (42 mg, yield 61%) as white solid.

### Procedure for Preparation of 11.

A 20 mL vial was charge with magnetic stirring bar and tert-butyl (4-(3-(2,4-dioxotetrahydropyrimidin-1(2H)-yl)-2-methylphenyl)but-3-yn-1-yl)carbamate (74 mg, 0.2 mmol). Next, HCl/dioxane (1.0 mL, 4M) was added by syringe. The mixture was stirred at room temperature for half-hour, TLC indicated that the reaction was completed. The reaction mixture was concentrated under high vacuum at room temperature to provide solid. The solid was dissolved by DMF (1.0 mL) in the same vial, tricthylamine (0.136 mL, 0.98 mmol) and acetic anhydride (0.021 mL, 0.22 mmol) were successively added. The reaction mixture stirred at room temperature until the staring material disappeared as indicated by TLC. The mixture was partitioned between ethyl acetate and saturated solution of sodium bicarbonate, the organic layer was washed with brine, dried over Na₂SO₄, and concentrated in vacuum. The residue was purified by column chromatography on silica to provide 11 (30 mg, yield 48%) as white solid.

### General Procedure for Preparation of 12A, 12B and 12C.

A 20 mL vial was charge with magnetic stirring bar and 6F (54 mg, 0.15 mmol). Next, HCl/dioxane (1.0 mL, 4M) was added by syringe. The mixture was stirred at room temperature for half-hour, TLC indicated that the reaction was completed. The reaction mixture was concentrated under high vacuum at room temperature to provide solid. The solid was dissolved by DMF (1.0 mL) in the same vial, N,N-diisopropylethylamine (DIPEA) (0.107 mL, 0.6 mmol), JQ-1 (carboxylic acid) (Purchase from MedChemExpress Part NO.: HY-78695) (60 mg, 0.15 mmol) and HATU (63 mg, 0.165 mmol) were successively added. The mixture was stirred at room temperature for half-hour, TLC indicated that the reaction was completed. The mixture was partitioned between ethyl acetate and saturated solution of sodium bicarbonate, the organic layer was washed with brine, dried over Na₂SO₄, and concentrated in vacuum. The residue was purified by column chromatography on silica to provide 12A (83 mg, yield 86%) as white solid.

### Procedure for Preparation of 6K-Me tert-butyl 2-(2-methyl-3-(3-methyl-2,4-dioxotetrahydropyrimidin-1(2H)-yl)phenoxy)acetate

A 20 mL vial was charge with magnetic stirring bar, compound 6K (100 mg, 0.3 mmol) and Cs₂CO₃ (108 mg, 0.33 mmol). Next, DMF (1.0 mL) and iodomethane (Mel) (0.023 mL, 0.36 mmol) was added by syringe. The mixture was stirred at room temperature until the staring material 6K disappeared as indicated by TLC. The mixture was partitioned between ethyl acetate and saturated solution of sodium bicarbonate, the organic layer was washed with brine, dried over Na₂SO₄, and concentrated in vacuum, the residue was directly used for next step reaction without further purification.

### General Procedure for Preparation of 13, 13NT and 14

A 20 mL vial was charge with magnetic stirring bar and 6K (51 mg, 0.15 mmol). Next DCM (0.5 mL) and TFA (0.75 mL) was added. The reaction mixture stirred at room temperature until the staring material compound 6K disappeared as indicated by TLC. The mixture was concentrated in vacuum to provide white solid. In the same vial, DMF (0.5 mL), 4-(N-Boc-amino)piperidine (30 mg, 0.15 mmol), DIPEA (0.107 mL, 0.6 mmol) and HATU (63 mg, 0.165 mmol) were successively added. The reaction mixture stirred at room temperature until the staring material disappeared as indicated by TLC. The mixture was partitioned between ethyl acetate and saturated solution of sodium carbonate, the organic layer was washed with brine, dried over Na₂SO₄, and concentrated in vacuum. The residue was treated by DCM (0.75 mL) and TFA (0.75 mL) at room temperature for half-hour, TLC indicated that the reaction was completed. The mixture was concentrated in high vacuum, in the same vial, DIPEA (0.107 mL, 0.6 mmol), JQ-1 (carboxylic acid) (60 mg, 0.15 mmol) and HATU (63 mg, 0.165 mmol) were successively added. The mixture was stirred at room temperature for half-hour, TLC indicated that the reaction was completed. The mixture was partitioned between ethyl acetate and saturated solution of sodium bicarbonate, the organic layer was washed with brine, dried over Na₂SO₄, and concentrated in vacuum. The residue was purified by column chromatography on silica to provide 13 (85 mg, yield 76%) as white solid.

### Compound Characterization Data

1-phenyldihydropyrimidine-2,4(1H,3H)-dione (2) ¹H NMR (400 MHZ, DMSO-d₆) δ 10.36 (s, 1H), 7.43-7.35 (m, 2H), 7.39-7.29 (m, 2H), 7.27-7.19 (m, 1H), 3.79 (t, J=6.7 Hz, 2H), 2.70 (t, J=6.7 Hz, 2H). ¹³C NMR (101 MHZ, DMSO-d₆) δ 170.6, 152.1, 142.1, 128.6, 125.8, 125.3, 44.5, 31.1. HPLC purity: 97.8%.

1-(2-hydroxyphenyl)dihydropyrimidine-2,4(1H,3H)-dione (3A) ¹H NMR (400 MHZ, DMSO-d₆) δ 7.45 (s, 1H), 7.34-7.26 (m, 2H), 7.24-7.17 (m, 1H), 7.15-7.07 (m, 1H), 6.91 (s, 1H), 4.00 (t, J=6.9 Hz, 2H), 2.53 (t, J=6.9 Hz, 3H). ¹³C NMR (101 MHz, DMSO-d₆) δ=171.6, 153.6, 141.9, 131.0, 123.8, 122.1, 109.6, 109.5, 38.5, 33.1. HRMS (ESI/[M+H]⁺) Calcd for [C₁₀H₁₀N₂O₃+H]⁺: 207.0764, found: 207.0765. HPLC purity: 98.6%.

1-(o-tolyl)dihydropyrimidine-2,4(1H,3H)-dione (3B) ¹H NMR (400 MHZ, DMSO-d₆) δ 10.33 (s, 1H), 7.33-7.18 (m, 4H), 3.82-3.71 (m, 1H), 3.55-3.45 (m, 1H), 2.86-2.60 (m, 2H), 2.19 (s, 3H). ¹³C NMR (101 MHz, DMSO-d₆) δ=170.8, 151.7, 140.9, 135.5, 130.6, 127.4, 127.2, 126.8, 44.6, 31.1, 17.5. HRMS (ESI/[M+H]⁺) Calcd for [C₁₁H₁₂N₂O₂+H]⁺: 205.0972, found: 205.0973. HPLC purity: 97.5%.

1-(2-methoxyphenyl)dihydropyrimidine-2,4(1H,3H)-dione (3C) ¹H NMR (400 MHZ, DMSO-d₆) δ 10.29 (s, 1H), 7.36-7.28 (m, 1H), 7.27-7.21 (m, 1H), 7.14-7.07 (m, 1H), 7.01-6.92 (m, 1H), 3.80 (s, 3H), 3.57 (t, J=6.7 Hz, 2H), 2.67 (t, J=6.6 Hz, 2H). ¹³C NMR (101 MHZ, DMSO-d₆) δ=170.8, 154.8, 152.1, 130.1, 129.2, 128.8, 120.5, 112.4, 55.7, 44.5, 31.1. HRMS (ESI/[M+H]⁺) Calcd for [C₁₁H₁₂N₂O₃+H]⁺: 221.0921, found: 221.0924. HPLC purity: 98.2%.

1-(2-ethylphenyl)dihydropyrimidine-2,4(1H,3H)-dione (3D) ¹H NMR (400 MHZ, DMSO-d₆) δ 10.34 (s, 1H), 7.36-7.23 (m, 4H), 3.84-3.72 (m, 1H), 3.52-3.41 (m, 1H), 2.83-2.62 (m, 2H), 2.54 (tt, J=7.6, 3.8 Hz, 2H), 1.15 (t, J=7.6 Hz, 3H). ¹³C NMR (101 MHZ, DMSO-d₆) δ 170.7, 152.2, 141.2, 140.4, 128.8, 127.8, 127.6, 126.8, 45.0, 31.1, 23.5, 14.4. HRMS (ESI/[M+H]+) Calcd for [C₁₂H₁₄N₂O₂+H]⁺: 219.1128, found: 219.1131. HPLC purity: 97.6%.

1-(2-chlorophenyl)dihydropyrimidine-2,4(1H,3H)-dione (3E) ¹H NMR (400 MHZ, DMSO-d₆) δ 10.47 (s, 1H), 7.61-7.54 (m, 1H), 7.53-7.46 (m, 1H), 7.45-7.34 (m, 2H), 3.77-3.66 (m, 1H), 3.65-3.54 (m, 1H), 2.77-2.69 (m, 2H). ¹³C NMR (101 MHz, DMSO-d₆) δ 170.6, 151.8, 139.0, 131.8, 130.1, 129.9, 129.3, 128.2, 44.3, 31.0. HRMS (ESI/[M+H]+) Calcd for [C₁₀H₉ClN₂O₂+H]⁺: 225.0425, found: 225.0425. HPLC purity: 95.8%.

1-(3-iodophenyl)dihydropyrimidine-2,4(1H,3H)-dione (4) ¹H NMR (400 MHZ, DMSO-d₆) δ 10.42 (s, 1H), 7.76-7.70 (m, 1H), 7.63-7.56 (m, 1H), 7.39-7.32 (m, 1H), 7.23-7.15 (m, 1H), 3.78 (t, J=6.6 Hz, 2H), 2.69 (t, J=6.6 Hz, 2H). ¹³C NMR (101 MHz, DMSO-d₆) δ 170.6, 152.1, 143.3, 134.4, 133.9, 130.5, 124.7, 94.0, 44.4, 31.0.

1-(3-hydroxyphenyl)dihydropyrimidine-2,4(1H,3H)-dione (4A) ¹H NMR (400 MHZ, DMSO-d₆) δ 10.31 (s, 1H), 9.54 (s, 1H), 7.20-7.12 (m, 1H), 6.76-6.69 (m, 2H), 6.67-6.60 (m, 1H), 3.73 (t, J=6.6 Hz, 2H), 2.68 (t, J=6.6 Hz, 2H). ¹³C NMR (101 MHz, DMSO-d₆) δ 170.6, 157.5, 152.0, 143.1, 129.3, 115.6, 112.9, 112.6, 44.6, 31.1. HRMS (ESI/[M+H]+) Calcd for [C₁₀H₁₀N₂O₃+H]⁺: 207.0764, found: 207.0764. HPLC purity: 95.4%.

1-(3-methoxyphenyl)dihydropyrimidine-2,4(1H,3H)-dione (4C) ¹H NMR (400 MHZ, DMSO-d₆) δ 10.35 (s, 1H), 7.33-7.24 (m, 1H), 6.94-6.86 (m, 2H), 6.86-6.78 (m, 1H), 3.77 (t, J=6.7 Hz, 2H), 3.75 (s, 3H), 2.69 (t, J=6.7 Hz, 2H). ¹³C NMR (101 MHz, DMSO-d₆) δ 170.6, 159.4, 152.1, 143.2, 129.3, 117.5, 111.4, 111.4, 55.2, 44.6, 31.1. HRMS (ESI/[M+H]⁺) Calcd for [C₁₁H₁₂N₂O₃+H]⁺: 221.0921, found: 221.0922. HPLC purity: 95.3%.

tert-butyl (3-(3-(2,4-dioxotetrahydropyrimidin-1(2H)-yl)phenyl)prop-2-yn-1-yl)carbamate (4F) ¹H NMR (400 MHZ, DMSO-d₆) δ 10.41 (s, 1H), 7.49-7.32 (m, 4H), 7.29-7.22 (m, 1H), 3.98 (d, J=5.8 Hz, 2H), 3.79 (t, J=6.6 Hz, 2H), 2.70 (t, J=6.6 Hz, 2H), 1.40 (s, 9H). ¹³C NMR (101 MHZ, DMSO-d₆) δ 170.6, 155.3, 152.1, 142.2, 129.0, 128.5, 128.0, 125.4, 122.6, 88.0, 80.9, 78.3, 44.3, 31.0, 30.1, 28.2. HRMS (ESI/[M+Na]⁺) Calcd for [C₁₈H₂₁N₃O₄+Na]⁺: 366.1424, found: 366.1420. HPLC purity: 98.9%.

tert-butyl 4-(3-(2,4-dioxotetrahydropyrimidin-1(2H)-yl)phenoxy)piperidine-1-carboxylate (4H) ¹H NMR (400 MHZ, DMSO-d₆) δ 10.35 (s, 1H), 7.32-7.23 (m, 1H), 6.98-6.93 (m, 1H), 6.92-6.82 (m, 2H), 4.60-4.49 (m, 1H), 3.77 (t, J=6.7 Hz, 2H), 3.70-3.60 (m, 2H), 3.24-3.14 (m, 2H), 2.69 (t, J=6.7 Hz, 2H), 1.98-1.85 (m, 2H), 1.57-1.47 (m, 2H), 1.40 (s, 8H). ¹³C NMR (101 MHz, DMSO-d₆) δ 170.6, 157.0, 153.9, 152.1, 143.3, 129.4, 117.7, 113.3, 113.2, 78.7, 71.9, 48.6, 44.6, 31.0, 30.3, 28.1. HRMS (ESI/[M+H]⁺) Calcd for [C₂₀H₂₇N₃O₅+H]⁺: 390.2023, found: 390.2022. HPLC purity: 95.6%.

tert-butyl 2-(3-(2,4-dioxotetrahydropyrimidin-1(2H)-yl)phenoxy)acetate (4K) ¹H NMR (400 MHZ, DMSO-d₆) δ 10.36 (s, 1H), 7.34-7.24 (m, 1H), 6.97-6.88 (m, 2H), 6.81-6.73 (m, 1H), 4.65 (s, 2H), 3.76 (t, J=6.6 Hz, 2H), 2.69 (t, J=6.6 Hz, 2H), 1.43 (s, 9H). ¹³C NMR (101 MHz, DMSO-d₆) δ 170.6, 167.7, 157.8, 152.0, 143.1, 129.3, 118.1, 112.1, 111.6, 81.4, 65.1, 44.6, 31.0, 27.7. HRMS (ESI/[M+H]⁺) Calcd for [C₁₆H₂₀N₂O₅+H]⁺: 321.1445, found: 321.1444. HPLC purity: 97.4%.

1-(4-hydroxyphenyl)dihydropyrimidine-2,4(1H,3H)-dione (5A) ¹H NMR (400 MHZ, DMSO-d₆) § 10.25 (s, 1H), 9.46 (s, 1H), 7.14-7.06 (m, 2H), 6.79-6.71 (m, 2H), 3.67 (t, J=6.7 Hz, 2H), 2.67 (t, J=6.7 Hz, 2H). ¹³C NMR (101 MHz, DMSO-d₆) δ 170.7, 155.6, 152.3, 133.5, 127.0, 115.2, 45.1, 31.1. HRMS (ESI/[M+H]+) Calcd for [C₁₀H₁₀N₂O₃+H]⁺: 207.0764, found: 207.0763. HPLC purity: 99.0%.

tert-butyl 2-(4-(2,4-dioxotetrahydropyrimidin-1(2H)-yl)phenoxy)acetate (5K) ¹H NMR (400 MHZ, DMSO-d₆) δ 10.30 (s, 1H), 7.28-7.19 (m, 2H), 6.95-6.86 (m, 2H), 4.65 (s, 2H), 3.72 (t, J=6.7 Hz, 2H), 2.69 (t, J=6.7 Hz, 2H), 1.44 (s, 9H). ¹³C NMR (101 MHz, DMSO-d₆) δ 170.6, 167.8, 155.6, 152.3, 135.4, 126.8, 114.5, 81.4, 65.1, 44.9, 31.1, 27.7. HRMS (ESI/[M+H]⁺) Calcd for [C₁₆H₂₀N₂O₅+H]⁺: 321.1445, found: 321.1448. HPLC purity: 98.8%.

1-(3-iodo-2-methylphenyl)dihydropyrimidine-2,4(1H,3H)-dione (6) ¹H NMR (400 MHZ, DMSO-d₆) δ 10.40 (s, 1H), 7.82 (dd, J=7.9, 1.2 Hz, 1H), 7.33 (dd, J=7.9, 1.2 Hz, 1H), 7.07-6.98 (m, 1H), 3.83-3.72 (m, 1H), 3.57-3.46 (m, 1H), 2.86-2.73 (m, 1H), 2.73-2.62 (m, 1H), 2.28 (s, 3H). ¹³C NMR (101 MHz, DMSO-d₆) δ=170.7, 151.8, 141.0, 138.8, 138.1, 128.6, 127.7, 102.2, 44.6, 31.0, 23.5.

1-(3-hydroxy-2-methylphenyl)dihydropyrimidine-2,4(1H,3H)-dione (6A) ¹H NMR (400 MHz, DMSO-d₆) δ 10.28 (s, 1H), 9.49 (s, 1H), 7.06-6.98 (m, 1H), 6.81-6.74 (m, 1H), 6.73-6.67 (m, 1H), 3.77-3.66 (m, 1H), 3.52-3.42 (m, 1H), 2.81-2.70 (m, 1H), 2.70-2.59 (m, 1H), 1.97 (s, 3H). ¹³C NMR (101 MHz, DMSO-d₆) δ 170.8, 156.0, 151.7, 141.9, 126.3, 122.3, 117.6, 113.7, 44.7, 31.1, 10.7. HRMS (ESI/[M+H]⁺) Calcd for [C₁₁H₁₂N₂O₃+H]⁺: 221.0921, found: 221.0922. HPLC purity: 98.0%.

tert-butyl (3-(3-(2,4-dioxotetrahydropyrimidin-1(2H)-yl)-2-methylphenyl)prop-2-yn-1-yl)carbamate (6F) ¹H NMR (400 MHZ, DMSO-d₆) δ 10.37 (s, 1H), 7.46-7.36 (m, 1H), 7.36-7.18 (m, 3H), 4.00 (d, J=5.8 Hz, 2H), 3.85-3.69 (m, 1H), 3.58-3.44 (m, 1H), 2.87-2.58 (m, 2H), 2.24 (s, 3H), 1.40 (s, 9H). ¹³C NMR (101 MHz, DMSO-d₆) δ 170.7, 155.4, 151.8, 141.3, 137.7, 130.7, 127.6, 126.7, 123.6, 91.9, 79.9, 78.2, 44.5, 31.1, 30.3, 28.2, 15.6. HRMS (ESI/[M+Na]⁺) Calcd for [C₁₉H₂₃N₃O₄+Na]⁺: 380.1581, found: 380.1575. HPLC purity: 98.6%.

tert-butyl-(3-(3-(2,4-dioxotetrahydropyrimidin-1(2H)-yl)-2-methylphenyl)propyl)carbamate (6G) ¹H NMR (400 MHZ, DMSO-d₆) δ 10.30 (s, 1H), 7.19-7.08 (m, 3H), 6.95-6.87 (m, 1H), 3.79-3.67 (m, 1H), 3.53-3.42 (m, 1H), 3.03-2.94 (m, 2H), 2.84-2.72 (m, 1H), 2.72-2.62 (m, 1H), 2.62-2.54 (m, 2H), 2.09 (s, 3H), 1.62 (p, J=7.0 Hz, 2H), 1.38 (s, 9H). ¹³C NMR (101 MHz, DMSO-d₆) δ 170.8, 155.7, 151.9, 141.4, 141.2, 133.6, 128.0, 126.2, 124.9, 77.4, 44.8, 31.1, 30.5, 30.1, 28.3, 13.2. HRMS (ESI/[M+H]⁺) Calcd for [C₁₉H₂₇N₃O₄+H]⁺: 362.2074, found: 362.2080. HPLC purity: 98.3%.

tert-butyl-4-(3-(2,4-dioxotetrahydropyrimidin-1(2H)-yl)-2-methylphenoxy)piperidine-1-carboxylate (6H) ¹H NMR (400 MHZ, DMSO-d₆) δ ¹H NMR (400 MHZ, DMSO) δ 10.32 (s, 1H), 7.23-7.14 (m, 1H), 7.04-6.97 (m, 1H), 6.90-6.84 (m, 1H), 4.59 (tt, J=7.3, 3.5 Hz, 1H), 3.80-3.69 (m, 1H), 3.66-3.54 (m, 2H), 3.54-3.44 (m, 1H), 3.33-3.22 (m, 2H), 2.83-2.61 (m, 2H), 2.02 (s, 3H), 1.96-1.84 (m, 2H), 1.66-1.52 (m, 2H), 1.42 (s, 9H). ¹³C NMR (101 MHZ, DMSO-d₆) δ 170.7, 155.4, 153.9, 151.7, 142.0, 126.6, 125.1, 119.3, 112.1, 78.7, 71.9, 44.7, 31.1, 30.4, 28.1, 10.8. HRMS (ESI/[M+H]⁺) Calcd for [C₂₁H₂₉N₃O₅+H]⁺: 404.2180, found: 404.2182. HPLC purity: 96.3%.

3′-(2,4-dioxotetrahydropyrimidin-1(2H)-yl)-2′-methyl-[1,1′-biphenyl]-4-carbaldehyde (61) ¹H NMR (400 MHZ, DMSO-d₆) δ 10.38 (s, 1H), 10.08 (s, 1H), 8.04-7.97 (m, 2H), 7.62-7.55 (m, 2H), 7.41-7.35 (m, 2H), 7.29-7.20 (m, 1H), 3.89-3.77 (m, 1H), 3.62-3.52 (m, 1H), 2.87-2.75 (m, 1H), 2.75-2.65 (m, 1H), 2.09 (s, 3H). ¹³C NMR (101 MHz, DMSO-d₆) δ 192.8, 170.7, 151.9, 147.0, 141.7, 141.5, 134.9, 133.1, 130.0, 129.5, 128.6, 127.2, 126.7, 44.7, 31.1, 15.3. HRMS (ESI/[M+H]⁺) Calcd for [C₁₈H₁₆N₂O₃+H]⁺: 309.1234, found: 309.1239. HPLC purity: 95.5%.

4-((3-(2,4-dioxotetrahydropyrimidin-1(2H)-yl)-2-methylphenoxy)methyl)benzaldehyde (6J) ¹H NMR (400 MHZ, DMSO-d₆) δ 10.34 (s, 1H), 10.02 (s, 1H), 7.99-7.92 (m, 2H), 7.73-7.67 (m, 2H), 7.25-7.16 (m, 1H), 7.04-6.97 (m, 1H), 6.94-6.88 (m, 1H), 5.26 (s, 2H), 3.81-3.70 (m, 1H), 3.55-3.45 (m, 1H), 2.85-2.73 (m, 1H), 2.73-2.62 (m, 1H), 2.09 (s, 3H). 13C NMR (101 MHz, DMSO-d₆) δ 192.8, 170.7, 156.5, 151.8, 144.2, 141.8, 135.6, 129.8, 127.6, 126.7, 124.3, 119.6, 110.8, 69.0, 44.7, 31.1, 10.8. HRMS (ESI/[M+H]⁺) Calcd for [C₁₉H₁₈N₂O₄+H]⁺: 339.1339, found: 339.1336. HPLC purity: 97.8%.

tert-butyl 2-(3-(2,4-dioxotetrahydropyrimidin-1(2H)-yl)-2-methylphenoxy)acetate (6K) ¹H NMR (400 MHZ, DMSO-d₆) δ 10.33 (s, 1H), 7.21-7.13 (m, 1H), 6.93-6.87 (m, 1H), 6.83-6.76 (m, 1H), 4.70 (s, 2H), 3.80-3.69 (m, 1H), 3.54-3.43 (m, 1H), 2.84-2.72 (m, 1H), 2.72-2.61 (m, 1H), 2.05 (s, 3H). ¹³C NMR (101 MHz, DMSO-d₆) δ 170.7, 167.8, 156.3, 151.7, 141.8, 126.5, 124.4, 119.8, 110.5, 81.4, 65.6, 44.7, 31.1, 27.7, 10.7. HRMS (ESI/[M+H]⁺) Calcd for [C₁₇H₂₂N₂O₅+H]⁺: 335.1601, found: 335.1604. HPLC purity: 96.4%.

tert-butyl-3-(3-(2,4-dioxotetrahydropyrimidin-1(2H)-yl)-2-methylphenyl)propanoate (6L) ¹H NMR (400 MHZ, Acetone-d₆) δ 7.22-7.11 (m, 3H), 3.92-3.81 (m, 1H), 3.70-3.59 (m, 1H), 2.98-2.91 (m, 2H), 2.90-2.73 (m, 2H), 2.57-2.46 (m, 2H), 2.22 (s, 3H), 1.42 (s, 9H). ¹³C NMR (101 MHZ, Acetone-d₆) δ=172.4, 170.9, 152.6, 142.4, 141.4, 135.0, 128.9, 127.2, 126.2, 80.4, 46.1, 36.2, 32.1, 29.5, 28.2, 13.8. HRMS (ESI/[M+H]⁺) Calcd for [C₁₈H₂₄N₂O₄+H]⁺: 333.1809, found: 333.1813. HPLC purity: 97.9%.

tert-butyl-(E)-3-(3-(2,4-dioxotetrahydropyrimidin-1(2H)-yl)-2-methylphenyl)acrylate (6M) ¹H NMR (400 MHZ, DMSO-d₆) δ 10.37 (s, 1H), 7.83 (d, J=15.8 Hz, 1H), 7.71-7.65 (m, 1H), 7.38-7.33 (m, 1H), 7.33-7.24 (m, 1H), 6.42 (d, J=15.8 Hz, 1H), 3.83-3.71 (m, 1H), 3.55-3.44 (m, 1H), 2.87-2.76 (m, 1H), 2.72-2.63 (m, 1H), 2.22 (s, 3H), 1.49 (s, 9H). ¹³C NMR (101 MHz, DMSO-d₆) δ 170.7, 165.4, 152.0, 141.6, 140.9, 135.1, 134.3, 129.0, 126.8, 125.9, 121.8, 80.2, 44.6, 31.0, 27.8, 13.7. HRMS (ESI/[M+H]⁺) Calcd for [C₁₈H₂₂N₂O₄+H]⁺: 331.1652, found: 331.1665. HPLC purity: 99.6%.

1-(5-iodo-2-methylphenyl)dihydropyrimidine-2,4(1H,3H)-dione (7) ¹H NMR (400 MHZ, DMSO-d₆) δ 10.42 (s, 1H), 7.67 (d, J=1.9 Hz, 1H), 7.58 (dd, J=8.0, 1.9 Hz, 1H), 7.09 (d, J=8.1 Hz, 1H), 3.82-3.71 (m, 1H), 3.55-3.44 (m, 1H), 2.84-2.60 (m, 2H), 2.13 (s, 3H). ¹³C NMR (101 MHz, DMSO-d₆) δ 170.8, 151.7, 142.4, 136.1, 135.8, 135.7, 132.6, 90.7, 44.4, 31.1, 17.1.

1-(5-hydroxy-2-methylphenyl)dihydropyrimidine-2,4(1H,3H)-dione (7A) ¹H NMR (400 MHz, DMSO-d₆) δ 10.28 (s, 1H), 9.35 (s, 1H), 7.07-7.01 (m, 1H), 6.68-6.60 (m, 2H), 3.77-3.66 (m, 1H), 3.52-3.41 (m, 1H), 2.87-2.55 (m, 2H), 2.05 (s, 3H). ¹³C NMR (101 MHz, DMSO-d₆) δ 170.8, 156.0, 151.6, 141.4, 131.0, 125.2, 114.5, 113.9, 44.5, 31.1, 16.6. HRMS (ESI/[M+H]⁺) Calcd for [C₁₁H₁₂N₂O₃+H]⁺: 221.0921, found: 221.0916. HPLC purity: 99.2%.

tert-butyl (3-(3-(2,4-dioxotetrahydropyrimidin-1(2H)-yl)-4-methylphenyl)prop-2-yn-1-yl)carbamate (7F) ¹H NMR (400 MHZ, DMSO-d₆) δ 10.30 (s, 1H), 7.51-7.10 (m, 4H), 3.97 (d, J=5.6 Hz, 2H), 3.82-3.71 (m, 1H), 3.54-3.43 (m, 1H), 2.80-2.60 (m, 2H), 2.18 (s, 3H), 1.39 (s, 9H). ¹³C NMR (101 MHz, DMSO-d₆) δ 171.1, 155.3, 152.1, 141.3, 136.5, 130.9, 130.2, 130.1, 120.7, 87.6, 80.7, 78.3, 44.5, 31.2, 30.1, 28.2, 17.4. HRMS (ESI/[M+Na]⁺) Calcd for [C₁₉H₂₃N₃O₄+Na]⁺: 380.1581, found: 380.1598. HPLC purity: 99.1%.

tert-butyl-(3-(3-(2,4-dioxotetrahydropyrimidin-1(2H)-yl)-4-methylphenyl)propyl)carbamate (7G) ¹H NMR (400 MHZ, DMSO-d₆) δ 7.17 (d, J=7.9 Hz, 1H), 7.05 (dd, J=7.8, 1.8 Hz, 1H), 6.98 (d, J=1.8 Hz, 1H), 5.97 (s, 1H), 4.68 (s, 1H), 3.82-3.71 (m, 1H), 3.63-3.53 (m, 1H), 3.17-3.07 (m, 2H), 2.77 (t, J=6.8 Hz, 2H), 2.59 (t, J=7.7 Hz, 2H), 2.20 (s, 3H), 1.77 (p, J=7.3 Hz, 2H), 1.43 (s, 9H). ¹³C NMR (101 MHz, DMSO-d₆) δ 171.3, 156.1, 152.9, 140.9, 140.2, 133.0, 131.3, 128.3, 126.9, 79.3, 77.4, 45.4, 40.0, 32.4, 31.6, 28.6, 17.6. HRMS (ESI/[M+H]⁺) Calcd for [C₁₉H₂₇N₃O₄+H]⁺: 362.2074, found: 362.2065. HPLC purity: 96.1%.

tert-butyl-4-(3-(2,4-dioxotetrahydropyrimidin-1(2H)-yl)-4-methylphenoxy)piperidine-1-carboxylate (7H) ¹H NMR (400 MHZ, DMSO-d₆) δ 10.32 (s, 1H), 7.16 (d, J=8.4 Hz, 1H), 6.91 (d, J=2.6 Hz, 1H), 6.86 (dd, J=8.4, 2.6 Hz, 1H), 4.50 (tt, J=7.8, 3.6 Hz, 1H), 3.81-3.70 (m, 1H), 3.70-3.60 (m, 2H), 3.54-3.43 (m, 1H), 3.22-3.12 (m, 2H), 2.81-2.63 (m, 2H), 2.09 (s, 3H), 1.93-1.84 (m, 2H), 1.58-1.45 (m, 2H), 1.40 (s, 9H). ¹³C NMR (101 MHz, DMSO-d₆) δ 170.8, 155.5, 153.9, 151.6, 141.7, 131.1, 127.4, 114.9, 114.8, 78.7, 72.0, 44.5, 31.1, 30.3, 28.1, 16.6. HRMS (ESI/[M+H]⁺) Calcd for [C₂₁H₂₉N₃O₅+H]⁺: 404.2180, found: 404.2196. HPLC purity: 96.4%.

3′-(2,4-dioxotetrahydropyrimidin-1(2H)-yl)-4′-methyl-[1,1′-biphenyl]-4-carbaldehyde (71) ¹H NMR (400 MHZ, DMSO-d₆) δ 10.40 (s, 1H), 10.05 (s, 1H), 8.03-7.96 (m, 2H), 7.96-7.89 (m, 2H), 7.75 (d, J=2.0 Hz, 1H), 7.67 (dd, J=7.9, 2.1 Hz, 1H), 7.42 (d, J=8.1 Hz, 1H), 3.93-3.82 (m, 1H), 3.67-3.54 (m, 1H), 2.87-2.67 (m, 2H), 2.24 (s, 3H). ¹³C NMR (101 MHZ, DMSO-d₆) δ 192.7, 170.8, 151.8, 144.9, 141.7, 137.4, 136.2, 135.1, 131.4, 130.2, 127.1, 126.0, 125.9, 44.6, 31.2, 17.3. HRMS (ESI/[M+H]⁺) Calcd for [C₁₈H₁₆N₂O₃+H]⁺: 309.1234, found: 309.1239. HPLC purity: 95.2%.

4-((3-(2,4-dioxotetrahydropyrimidin-1(2H)-yl)-4-methylphenoxy)methyl)benzaldehyde (7J) ¹H NMR (400 MHZ, DMSO-d₆) δ 10.34 (s, 1H), 10.01 (s, 1H), 7.97-7.90 (m, 2H), 7.70-7.63 (m, 2H), 7.18 (d, J=8.5 Hz, 1H), 7.01 (d, J=2.7 Hz, 1H), 6.92 (dd, J=8.4, 2.7 Hz, 1H), 5.20 (s, 2H), 3.82-3.71 (m, 1H), 3.54-3.44 (m, 1H), 2.82-2.62 (m, 2H), 2.10 (s, 3H). ¹³C NMR (101 MHz, DMSO-d₆) δ 192.8, 170.8, 156.8, 151.6, 143.9, 141.6, 135.6, 131.1, 129.7, 127.8, 127.8, 113.8, 113.8, 68.7, 44.5, 31.1, 16.6. HRMS (ESI/[M+H]⁺) Calcd for [C₁₉H₁₈N₂O₄+H]⁺: 339.1339, found: 339.1334. HPLC purity: 98.4%.

tert-butyl-2-(3-(2,4-dioxotetrahydropyrimidin-1(2H)-yl)-4-methylphenoxy)acetate (7K) ¹H NMR (400 MHZ, DMSO-d₆) δ 10.32 (s, 1H), 7.17 (d, J=8.4 Hz, 1H), 6.87 (d, J=2.7 Hz, 1H), 6.78 (dd, J=8.4, 2.7 Hz, 1H), 4.62 (s, 2H), 3.81-3.70 (m, 1H), 3.53-3.43 (m, 1H), 2.86-2.57 (m, 2H), 2.10 (s, 3H), 1.43 (s, 9H). ¹³C NMR (101 MHz, DMSO-d₆) δ 170.7, 167.8, 156.3, 151.6, 141.5, 131.0, 127.9, 113.5, 113.4, 81.4, 65.2, 44.5, 31.1, 27.7, 16.6. HRMS (ESI/[M+H]⁺) Calcd for [C₁₇H₂₂N₂O₅+H]⁺: 335.1601, found: 335.1592. HPLC purity: 97.5%.

tert-butyl-3-(3-(2,4-dioxotetrahydropyrimidin-1(2H)-yl)-4-methylphenyl)propanoate (7L) ¹H NMR (400 MHZ, CDCl₃) δ 7.43 (s, 1H), 7.21 (d, J=7.8 Hz, 1H), 7.11 (dd, J=7.8, 1.8 Hz, 1H), 7.02 (d, J=1.8 Hz, 1H), 3.86-3.74 (m, 1H), 3.68-3.57 (m, 1H), 2.93-2.79 (m, 4H), 2.53 (t, J=7.1 Hz, 2H), 2.23 (s, 3H), 1.42 (s, 9H). ¹³C NMR (101 MHz, DMSO-d₆) δ 171.5, 170.8, 151.6, 140.7, 139.3, 133.0, 130.4, 127.3, 126.8, 79.7, 44.6, 36.1, 31.2, 29.8, 27.7, 17.1. HRMS (ESI/[M+H]⁺) Calcd for [C₁₈H₂₄N₂O₄+H]⁺: 333.1809, found: 333.1802. HPLC purity: 97.9%.

tert-butyl-(E)-3-(3-(2,4-dioxotetrahydropyrimidin-1(2H)-yl)-4-methylphenyl)acrylate (7M) ¹H NMR (400 MHZ, DMSO-d₆) δ 10.38 (s, 1H), 7.69 (d, J=1.8 Hz, 1H), 7.57-7.47 (m, 2H), 7.31 (d, J=8.0 Hz, 1H), 6.52 (d, J=15.9 Hz, 1H), 3.86-3.75 (m, 1H), 3.58-3.48 (m, 1H), 2.84-2.66 (m, 2H), 2.20 (s, 3H), 1.48 (s, 9H). ¹³C NMR (101 MHZ, DMSO-d₆) δ 170.8, 165.5, 151.7, 142.7, 141.5, 138.1, 133.1, 131.1, 127.5, 126.7, 119.7, 79.9, 44.6, 31.1, 27.8, 17.5. HRMS (ESI/[M+H]⁺) Calcd for [C₁₈H₂₂N₂O₄+H]⁺: 331.1652, found: 331.1647. HPLC purity: 98.5%.

1-(4-hydroxy-2-methylphenyl)dihydropyrimidine-2,4(1H,3H)-dione (8A) ¹H NMR (400 MHz, DMSO-d₆) δ ¹H NMR (400 MHZ, DMSO) δ 10.23 (s, 1H), 9.40 (s, 1H), 7.01 (d, J=8.5 Hz, 1H), 6.64 (d, J=2.8 Hz, 1H), 6.60 (dd, J=8.4, 2.8 Hz, 1H), 3.71-3.60 (m, 1H), 3.50-3.39 (m, 1H), 2.79-2.59 (m, 2H), 2.08 (s, 3H). ¹³C NMR (101 MHZ, DMSO-d₆) δ 170.8, 156.4, 152.0, 136.5, 132.3, 128.2, 116.8, 113.3, 44.9, 31.2, 17.5. HRMS (ESI/[M+H]⁺) Calcd for [C₁₁H₁₂N₂O₃+H]⁺: 221.0921, found: 221.0916. HPLC purity: 97.9%.

tert-butyl-4-(4-(2,4-dioxotetrahydropyrimidin-1(2H)-yl)-3-methylphenoxy)piperidine-1-carboxylate (8H) ¹H NMR (400 MHZ, DMSO-d₆) δ 10.28 (s, 1H), 7.14 (d, J=8.6 Hz, 1H), 6.88 (d, J=2.9 Hz, 1H), 6.82 (dd, J=8.6, 2.9 Hz, 1H), 4.54 (tt, J=7.8, 3.6 Hz, 1H), 3.73-3.60 (m, 3H), 3.52-3.42 (m, 1H), 3.23-3.14 (m, 2H), 2.80-2.60 (m, 2H), 2.14 (s, 3H), 1.94-1.84 (m, 2H), 1.58-1.45 (m, 2H), 1.41 (s, 9H). ¹³C NMR (101 MHZ, DMSO-d₆) δ 170.8, 155.8, 153.9, 151.9, 136.9, 133.9, 128.3, 117.5, 113.7, 78.7, 71.9, 44.8, 31.1, 30.4, 28.1, 17.6. HRMS (ESI/[M+H]⁺) Calcd for [C₂₁H₂₉N₃O₅+H]⁺: 404.2180, found: 404.2169. HPLC purity: 96.3%.

4-((4-(2,4-dioxotetrahydropyrimidin-1(2H)-yl)-3-methylphenoxy)methyl)benzaldehyde (8J) ¹H NMR (400 MHZ, DMSO-d₆) δ 10.29 (s, 1H), 10.01 (s, 1H), 7.97-7.91 (m, 2H), 7.70-7.64 (m, 2H), 7.17 (d, J=8.6 Hz, 1H), 6.96 (d, J=2.9 Hz, 1H), 6.88 (dd, J=8.6, 3.0 Hz, 1H), 5.23 (s, 2H), 3.75-3.64 (m, 1H), 3.52-3.41 (m, 1H), 2.81-2.60 (m, 2H), 2.15 (s, 3H). ¹³C NMR (101 MHZ, DMSO-d₆) δ 192.8, 170.8, 156.9, 151.9, 144.1, 136.9, 135.6, 134.2, 129.7, 128.4, 127.7, 116.5, 112.8, 68.6, 44.8, 31.1, 17.6. HRMS (ESI/[M+H]⁺) Calcd for [C₁₉H₁₈N₂O₄+H]⁺: 339.1339, found: 339.1335. HPLC purity: 98.5%.

tert-butyl-2-(4-(2,4-dioxotetrahydropyrimidin-1(2H)-yl)-3-methylphenoxy)acetate (8K) ¹H NMR (400 MHZ, DMSO-d₆) δ 10.28 (s, 1H), 7.16 (d, J=8.6 Hz, 1H), 6.81 (d, J=3.0 Hz, 1H), 6.75 (dd, J=8.6, 3.0 Hz, 1H), 4.64 (s, 2H), 3.75-3.64 (m, 1H), 3.52-3.42 (m, 1H), 2.81-2.60 (m, 2H), 2.14 (s, 3H), 1.44 (s, 9H). ¹³C NMR (101 MHZ, DMSO-d₆) δ170.8, 167.8, 156.5, 151.9, 136.8, 134.3, 128.2, 116.2, 112.4, 81.4, 65.0, 44.8, 31.1, 27.7, 17.6. HRMS (ESI/[M+H]⁺) Calcd for [C₁₇H₂₂N₂O₅+H]⁺: 335.1601, found: 335.1593. HPLC purity: 95.8%.

1-(2-methyl-3-(2-oxo-2-(piperidin-1-yl)ethoxy)phenyl)dihydropyrimidine-2,4(1H,3H)-dione (9) ¹H NMR (400 MHZ, DMSO-d₆) δ 10.32 (s, 1H), 7.20-7.12 (m, 1H), 6.91-6.80 (m, 2H), 3.80-3.69 (m, 1H), 3.53-3.35 (m, 5H), 2.84-2.61 (m, 2H), 2.04 (s, 3H), 1.64-1.39 (m, 6H). ¹³C NMR (101 MHz, DMSO-d₆) 170.7, 165.4, 156.6, 151.7, 141.7, 126.5, 124.1, 119.4, 110.6, 66.7, 45.2, 44.7, 42.2, 31.1, 26.0, 25.3, 24.0, 10.8. HRMS (ESI/[M+H]⁺) Calcd for [C₁₈H₂₃N₃O₄+H]⁺: 346.1761, found: 346.1760. HPLC purity: 98.9%.

2-(3-(2,4-dioxotetrahydropyrimidin-1(2H)-yl)-2-methylphenoxy)-N-ethylacetamide (10) ¹H NMR (400 MHz, DMSO-d₆) δ 10.33 (s, 1H), 8.08-7.91 (m, 1H), 7.23-7.14 (m, 1H), 6.95-6.88 (m, 1H), 6.86-6.79 (m, 1H), 4.48 (s, 2H), 3.80-3.69 (m, 1H), 3.54-3.43 (m, 1H), 3.23-3.12 (m, 2H), 2.84-2.62 (m, 2H), 2.09 (s, 3H), 1.05 (t, J=7.2 Hz, 3H). ¹³C NMR (101 MHZ, DMSO-d₆) δ 170.7, 167.3, 156.5, 151.8, 141.8, 126.6, 124.6, 119.9, 110.8, 67.7, 44.7, 33.3, 31.1, 14.8, 10.9. HRMS (ESI/[M+H]⁺) Calcd for [C₁₅H₁₉N₃O₄+H]⁺: 306.1448, found: 306.1444. HPLC purity: 99.1%.

N-(4-(3-(2,4-dioxotetrahydropyrimidin-1(2H)-yl)-2-methylphenyl)but-3-yn-1-yl)acetamide (11) ¹H NMR (400 MHZ, DMSO-d₆) δ 10.36 (s, 1H), 8.11-8.04 (m, 1H), 7.38-7.32 (m, 1H), 7.30-7.18 (m, 2H), 3.82-3.71 (m, 1H), 3.55-3.45 (m, 1H), 3.25 (t, J=6.9 Hz, 2H), 2.85-2.72 (m, 1H), 2.75-2.62 (m, 1H), 2.59 (t, J=6.9 Hz, 2H), 2.24 (s, 3H), 1.82 (s, 3H). ¹³C NMR (101 MHZ, DMSO-d₆) δ 170.7, 169.3, 151.8, 141.2, 137.5, 130.8, 127.3, 126.6, 124.2, 92.7, 79.7, 44.5, 37.9, 31.1, 22.6, 19.9, 15.7. HRMS (ESI/[M+H]⁺) Calcd for [C₁₇H₁₉N₃O₃+H]⁺: 314.1499, found: 314.1491. HPLC purity: 95.4%.

(S)-2-(4-(4-chlorophenyl)-2,3,9-trimethyl-6H-thieno[3,2-f][1,2,4]triazolo[4,3-a][1,4]diazepin-6-yl)-N-(3-(3-(2,4-dioxotetrahydropyrimidin-1(2H)-yl)-2-methylphenyl)prop-2-yn-1-yl)acetamide (12A) ¹H NMR (400 MHZ, CDCl₃) δ 8.07 (d, J=5.3 Hz, 1H), 7.42-7.36 (m, 3H), 7.35-7.30 (m, 1H), 7.25-7.13 (m, 4H), 4.68-4.60 (m, 1H), 4.50-4.38 (m, 1H), 4.29-4.17 (m, 1H), 3.81-3.68 (m, 1H), 3.63-3.40 (m, 3H), 2.84-2.75 (m, 2H), 2.65 (d, J=1.5 Hz, 3H), 2.39 (s, 3H), 2.28 (d, J=3.7 Hz, 3H), 1.68-1.62 (m, 3H). ¹³C NMR (101 MHZ, CDCl₃) δ 13C NMR (101 MHZ, CDCl₃) δ 170.4, 169.7, 164.2, 155.6, 151.6, 150.1, 140.1, 138.2, 136.8, 136.7, 132.4, 132.3, 131.0, 131.0, 130.5, 130.0, 128.8, 127.4, 126.9, 124.9, 90.1, 81.4, 54.5, 45.3, 39.1, 31.5, 30.2, 16.2, 14.5, 13.2, 11.9. HRMS (ESI/[M+H]⁺) Calcd for [C₃₃H₃₀ClN₇O₃S+H]⁺: 640.1892, found: 640.1883. HPLC purity: 96.0%.

(S)-2-(4-(4-chlorophenyl)-2,3,9-trimethyl-6H-thieno[3,2-f][1,2,4]triazolo[4,3-a][1,4]diazepin-6-yl)-N-(4-(3-(2,4-dioxotetrahydropyrimidin-1(2H)-yl)-2-methylphenyl)but-3-yn-1-yl)acetamide (12B) ¹H NMR (400 MHZ, MeOD-d₄) δ 7.50-7.41 (m, 2H), 7.39-7.27 (m, 3H), 7.26-7.13 (m, 2H), 4.69 (dd, J=8.8, 5.2 Hz, 1H), 3.86-3.69 (m, 1H), 3.65-3.42 (m, 4H), 3.38-3.32 (m, 2H), 2.91-2.73 (m, 3H), 2.71 (s, 3H), 2.43 (s, 3H), 2.31 (s, 3H), 1.73-1.63 (m, 3H). ¹³C NMR (101 MHZ, MeOD-d₄) δ 172.8, 172.8, 166.5, 156.9, 154.1, 152.4, 141.9, 139.2, 138.2, 137.8, 133.7, 133.3, 132.9, 132.2, 132.0, 131.4, 129.8, 128.2, 127.8, 126.6, 93.2, 81.0, 55.0, 46.3, 39.9, 38.5, 32.1, 21.0, 16.3, 14.4, 13.0, 11.6. HRMS (ESI/[M+H]⁺) Calcd for [C₃₄H₃₂ClN₇O₃S+H]⁺: 654.2049, found: 654.2031. HPLC purity: 95.6%.

(S)-2-(4-(4-chlorophenyl)-2,3,9-trimethyl-6H-thieno[3,2-f][1,2,4]triazolo[4,3-a][1,4]diazepin-6-yl)-N-(5-(3-(2,4-dioxotetrahydropyrimidin-1(2H)-yl)-2-methylphenyl)pent-4-yn-1-yl)acetamide (12C) ¹H NMR (101 MHZ, MeOD-d₄) δ 7.51-7.44 (m, 2H), 7.44-7.29 (m, 3H), 7.25-7.15 (m, 2H), 4.69 (dd, J=9.0, 5.3 Hz, 1H), 3.87-3.76 (m, 1H), 3.67-3.57 (m, 1H), 3.54-3.37 (m, 3H), 3.36-3.26 (m, 1H), 2.93-2.70 (m, 2H), 2.73 (s, 3H), 2.59 (t, J=7.1 Hz, 2H), 2.45 (s, 3H), 2.32 (d, J=11.6 Hz, 3H), 1.90 (p, J=6.9 Hz, 2H), 1.73-1.67 (m, 3H). 13C NMR (101 MHZ, MeOD-d₄) δ 172.9, 172.7, 166.6, 156.9, 154.2, 152.5, 141.9, 139.0, 138.3, 137.7, 133.8, 133.4, 132.8, 132.2, 132.0, 131.5, 129.9, 128.0, 127.8, 126.8, 95.1, 80.5, 55.1, 46.3, 39.6, 38.5, 32.1, 29.7, 17.7, 16.3, 14.4, 12.9, 11.5. HRMS (ESI/[M+H]⁺) Calcd for [C₃₅H₃₄ClN₇O₃S+H]⁺: 668.2205, found: 668.2195. HPLC purity: 96.7%.

(S)-2-(4-(4-chlorophenyl)-2,3,9-trimethyl-6H-thieno[3,2-f][1,2,4]triazolo[4,3-a][1,4]diazepin-6-yl)-N-(1-(2-(3-(2,4-dioxotetrahydropyrimidin-1(2H)-yl)-2-methylphenoxy)acetyl)piperidin-4-yl)acetamide (13) ¹H NMR (400 MHZ, Acetone-d₆) δ 7.53-7.38 (m, 4H), 7.22-7.10 (m, 1H), 7.04-6.85 (m, 2H), 4.98-4.76 (m, 2H), 4.68-4.57 (m, 1H), 4.41-4.20 (m, 1H), 4.12-3.79 (m, 3H), 3.77-3.59 (m, 1H), 3.43-3.16 (m, 3H), 2.89-2.72 (m, 3H), 2.61 (s, 3H), 2.45 (s, 3H), 2.15 (s, 3H), 2.01-1.78 (m, 2H), 1.70 (s, 3H), 1.62-1.34 (m, 2H). ¹³C NMR (101 MHZ, Acetone-d₆) δ 171.0, 170.1, 166.6, 164.2, 157.9, 156.5, 152.5, 150.6, 142.9, 138.3, 136.7, 133.6, 131.7, 131.2, 131.1, 131.0, 129.3, 127.5, 125.8, 120.5, 111.6, 68.4, 55.2, 47.1, 46.0, 44.5, 41.4, 39.1, 33.1, 32.6, 32.1, 14.5, 13.0, 11.8, 11.3. HRMS (ESI/[M+H]⁺) Calcd for [C₃₇H₃₉ClN₈O₅S+H]⁺: 743.2525, found: 743.2504. HPLC purity: 96.0%.

(S)-2-(4-(4-chlorophenyl)-2,3,9-trimethyl-6H-thieno[3,2-f][1,2,4]triazolo[4,3-a][1,4]diazepin-6-yl)-N-(1-(2-(2-methyl-3-(3-methyl-2,4-dioxotetrahydropyrimidin-1(2H)-yl)phenoxy)acetyl)piperidin-4-yl)acetamide (13NT) ¹H NMR (400 MHZ, Acetone-d₆) δ 7.52-7.45 (m, 2H), 7.44-7.38 (m, 2H), 7.21-7.13 (m, 1H), 6.98-6.88 (m, 2H), 5.02-4.73 (m, 2H), 4.69-4.57 (m, 1H), 4.31 (d, J=11.3 Hz, 1H), 4.10-3.92 (m, 2H), 3.90-3.76 (m, 1H), 3.67-3.56 (m, 1H), 3.45-3.18 (m, 3H), 3.10 (s, 3H), 2.98-2.75 (m, 3H), 2.61 (s, 3H), 2.44 (s, 3H), 2.15 (s, 3H), 2.02-1.78 (m, 2H), 1.70 (s, 3H), 1.64-1.38 (m, 2H). ¹H NMR (400 MHZ, Acetone-d₆) δ 170.3, 170.1, 166.6, 164.2, 157.9, 156.5, 153.6, 150.6, 143.6, 138.2, 136.7, 133.6, 131.7, 131.2, 131.1, 131.0, 129.3, 127.5, 125.7, 120.5, 111.6, 68.5, 55.2, 47.0, 44.8, 44.6, 41.5, 39.2, 32.9, 32.5, 32.2, 27.6, 14.5, 13.0, 11.8, 11.3. HRMS (ESI/[M+H]⁺) Calcd for [C₃₈H₄₁ClN₈O₅S+H]⁺: 757.2682, found: 757.2662. HPLC purity: 97.2%.

(S)—N-(2-(1-(2-(4-(4-chlorophenyl)-2,3,9-trimethyl-6H-thieno[3,2-f][1,2,4]triazolo[4,3-a][1,4]diazepin-6-yl)acetyl)piperidin-4-yl)ethyl)-2-(3-(2,4-dioxotetrahydropyrimidin-1(2H)-yl)-2-methylphenoxy)acetamide (14) ¹H NMR (400 MHZ, Acetone-d₆) δ 9.19 (t, J=4.3 Hz, 1H), 7.53-7.45 (m, 3H), 7.45-7.38 (m, 2H), 7.27-7.18 (m, 1H), 7.01-6.95 (m, 1H), 6.92-6.86 (m, 1H), 4.71 (t, J=6.6 Hz, 1H), 4.56-4.45 (m, 3H), 4.23-4.15 (m, 1H), 3.95-3.83 (m, 1H), 3.70-3.55 (m, 2H), 3.48-3.37 (m, 1H), 3.40-3.31 (m, 2H), 3.16-3.03 (m, 1H), 2.89-2.69 (m, 2H), 2.61 (s, 3H), 2.58-2.47 (m, 1H), 2.44 (s, 3H), 2.16 (s, 3H), 1.92-1.41 (m, 8H), 1.23 (s, 1H), 1.13-0.93 (m, 1H). ¹³C NMR (101 MHZ, Acetone-d₆) δ 170.9, 168.9, 168.5, 163.9, 157.5, 156.7, 152.5, 150.4, 143.1, 138.4, 136.6, 133.6, 131.6, 131.2, 131.1, 131.0, 129.3, 127.7, 126.0, 121.0, 111.6, 68.9, 55.6, 46.5, 46.0, 42.6, 36.9, 36.9, 36.0, 34.5, 33.5, 32.7, 32.1, 14.5, 13.0, 11.7, 11.3. HRMS (ESI/[M+H]⁺) Calcd for [C₃₉H₄₃ClN₈O₅S+H]⁺: 771.2838, found: 771.2822. HPLC purity: 95.4%.

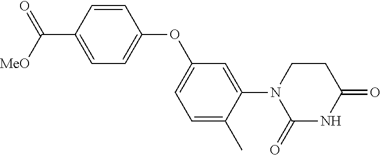 [SMILES: COC(=O)c1ccc(Oc2ccc(C)c(N3CCC(=O)NC3=O)c2)cc1]

(17) ¹H NMR (400 MHZ, CDCl₃) § 8.01 (d, J=8.9 Hz, 2H), 7.44 (s, 1H), 7.30 (d, J=8.4 Hz, 1H), 7.05-6.95 (m, 3H), 6.91 (d, J=2.5 Hz, 1H), 3.90 (s, 3H), 3.85-3.60 (m, 1H), 2.94-2.71 (m, 1H), 2.83 (m, 2H), 2.27 (s, 3H); ¹³C NMR (101 MHZ, CDCl₃) δ 169.2, 166.5, 161.3, 154.6, 151.0, 140.8, 132.5, 131.8, 131.6, 124.9, 119.8, 118.3, 117.5, 52.1, 45.1, 31.5, 17.4. calcd for [C₁₉H₁₈N₂O₅+H]⁺, 355.1; found, 355.1.

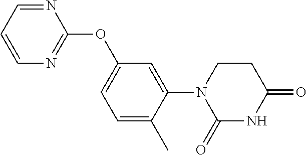 [SMILES: Cc1ccc(Oc2ncccn2)cc1N1CCC(=O)NC1=O]

(18) ¹H NMR (400 MHZ, CDCl₃) δ 8.55 (d, J=4.8 Hz, 2H), 7.53 (s, 1H), 7.35 (d, J=8.4 Hz, 1H), 7.15 (dd, J=8.3, 2.4 Hz, 1H), 7.08 (d, J=2.4 Hz, 1H), 7.05 (t, J=4.8 Hz, 1H), 3.90-3.79 (m, 1H), 3.74-3.65 (m, 1H), 2.88-2.78 (m, 2H), 2.29 (s, 3H); ¹³C NMR (101 MHz, CDCl₃) δ 169.4, 165.2, 159.8, 151.5, 151.0, 140.3, 132.7, 132.0, 121.8, 120.19, 116.4, 45.2, 31.5, 17.5. calcd for [C₁₅H₁₄N₄O₃+H]⁺, 299.1; found, 299.1.

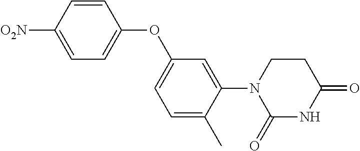 [SMILES: Cc1ccc(Oc2ccc([N+](=O)[O-])cc2)cc1N1CCC(=O)NC1=O]

(19) ¹H NMR (400 MHZ, CDCl₃) δ 8.29-8.08 (m, 2H), 7.65 (s, 1H), 7.35 (d, J=8.4 Hz, 1H), 7.12-6.99 (m, 3H), 6.96 (d, J=2.5 Hz, 1H), 3.87-3.77 (m, 1H), 3.67 (m, 1H), 2.85 (q, J=7.0 Hz, 2H), 2.29 (s, 3H). ¹³C NMR (101 MHZ, CDCl₃) δ 169.2, 162.9, 153.5, 151.1, 142.9, 141.0, 132.8, 132.7, 126.0, 120.3, 119.0, 117.3, 45.1, 31.5, 17.5. calcd for [C₁₇H₁₅N₃O₅+H]⁺, 342.1; found, 342.1.

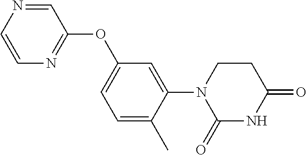 [SMILES: Cc1ccc(Oc2cnccn2)cc1N1CCC(=O)NC1=O]

(20) ¹H NMR (400 MHZ, CDCl₃) δ 8.44 (d, J=1.4 Hz, 1H), 8.28 (d, J=2.7 Hz, 1H), 8.09 (dd, J=2.7, 1.4 Hz, 1H), 7.73 (s, 1H), 7.34 (d, J=8.4 Hz, 1H), 7.10 (dd, J=8.4, 2.5 Hz, 1H), 7.05 (d, J=2.5 Hz, 1H), 3.94-3.77 (m, 1H), 3.68 (m, 1H), 2.92-2.76 (m, 2H), 2.28 (s, 3H). ¹³C NMR (101 MHz, CDCl₃) δ 169.4, 159.9, 151.6, 151.1, 141.0, 140.5, 138.8, 135.9, 132.7, 132.2, 121.3, 119.8, 45.1, 31.5, 17.5. calcd for [C₁₅H₁₄N₄O₃+H]⁺, 299.1; found, 299.1.

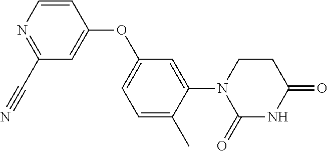 [SMILES: Cc1ccc(Oc2ccnc(C#N)c2)cc1N1CCC(=O)NC1=O]

(21) ¹H NMR (400 MHZ, CDCl₃) δ 8.54 (d, J=5.8 Hz, 1H), 7.62 (s, 1H), 7.40 (d, J=8.4 Hz, 1H), 7.24 (d, J=2.5 Hz, 1H), 7.07-7.01 (m, 2H), 6.98 (d, J=2.5 Hz, 1H), 3.87-3.76 (m, 1H), 3.69 (m, 1H), 2.86 (q, J=6.9 Hz, 2H), 2.32 (s, 3H). ¹³C NMR (101 MHZ, CDCl₃) δ 169.1, 165.0, 152.8, 151.6, 151.1, 141.3, 135.4, 134.0, 133.1, 120.6, 119.53, 117.0, 116.8, 114.7, 45.1, 31.4, 17.6. calcd for [C₁₇H₁₄N₄O₃+H]⁺, 323.1; found, 323.1.

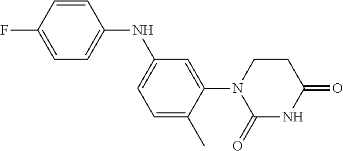 [SMILES: Cc1ccc(Nc2ccc(F)cc2)cc1N1CCC(=O)NC1=O]

(22) ¹H NMR (400 MHZ, CDCl₃) δ 7.43 (s, 1H), 7.15 (d, J=8.3 Hz, 1H), 7.08-6.93 (m, 4H), 6.88 (dd, J=8.3, 2.5 Hz, 1H), 6.79 (d, J=2.4 Hz, 1H), 5.56 (s, 1H), 3.77 (m, 1H), 3.65 (m, 1H), 2.90-2.73 (m, 2H), 2.19 (s, 3H). ¹³C NMR (101 MHz, CDCl₃) δ 169.5, 158.2 (d, J=240.6 Hz), 151.3, 143.3, 140.3, 138.5 (d, J=2.5 Hz), 132.0, 126.9, 120.7 (d, J=8.0 Hz), 116.9, 116.1 (d, J=22.6 Hz), 115.0, 45.1, 31.5, 17.1. calcd for [C₁₇H₁₆FN₃O₂+H]⁺, 314.1; found, 314.1.

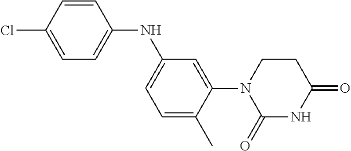 [SMILES: Cc1ccc(Nc2ccc(Cl)cc2)cc1N1CCC(=O)NC1=O]

(23) ¹H NMR (400 MHZ, CDCl₃) δ 7.42 (s, 1H), 7.24-7.15 (m, 3H), 6.99-6.93 (m, 3H), 6.87 (d, J=2.4 Hz, 1H), 5.65 (s, 1H), 3.77 (m, 1H), 3.65 (m, 1H), 2.83 (q, J=6.5 Hz, 2H), 2.21 (s, 3H). ¹³C NMR (101 MHz, DMSO) δ 171.2, 152.2, 143.2, 141.9, 141.9, 131.6, 129.4, 127.5, 122.9, 117.9, 117.4, 116.8, 44.9, 31.6, 17.2. calcd for [C₁₇H₁₆ClN₃O₂+H]⁺, 330.1; found, 330.1.

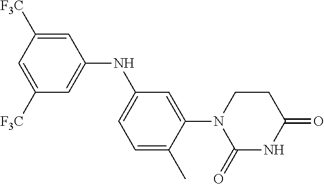 [SMILES: Cc1ccc(Nc2cc(C(F)(F)F)cc(C(F)(F)F)c2)cc1N1CCC(=O)NC1=O]

(24) ¹H NMR (400 MHZ, CDCl₃) δ 7.48 (s, 1H), 7.30 (m, 4H), 7.07 (dd, J=8.2, 2.4 Hz, 1H), 6.94 (d, J=2.3 Hz, 1H), 6.07 (s, 1H), 3.95-3.75 (m, 1H), 3.68 (q, J=6.3 Hz, 1H), 2.91-2.78 (m, 2H), 2.26 (s, 3H). ¹³C NMR (101 MHZ, DMSO) δ 171.2, 152.3, 146.9, 142.1, 139.5, 132.1, 131.8 (q, J=32.5), 130.5, 125.2, 122.5, 119.9, 114.2, 110.9, 44.9, 31.6, 17.4. calcd for [C₁₉H₁₅F₆N₃O₂+H]⁺, 432.1; found, 432.1.

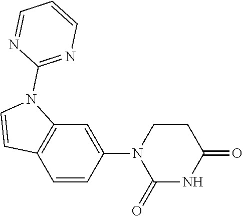 [SMILES: O=C1CCN(c2ccc3ccn(-c4ncccn4)c3c2)C(=O)N1]

(25) ¹H NMR (400 MHZ, CDCl₃) δ 8.80 (d, J=2.0 Hz, 1H), 8.71 (d, J=4.8 Hz, 2H), 8.33 (d, J=3.7 Hz, 1H), 7.55 (s, 1H), 7.26 (s, 1H), 7.17 (dd, J=8.3, 2.0 Hz, 1H), 7.08 (t, J=4.8 Hz, 1H), 6.70 (d, J=3.6 Hz, 1H), 3.97 (t, J=6.7 Hz, 2H), 2.89 (t, J=6.7 Hz, 2H). ¹³C NMR (101 MHz, CDCl₃) δ 169.7, 158.2, 157.6, 152.1, 137.0, 135.3, 130.5, 127.2, 121.3, 120.0, 116.5, 114.1, 106.7, 46.1, 31.6. calcd for [C₁₆H₁₃N₅O₂+H]⁺, 308.1; found, 308.1.

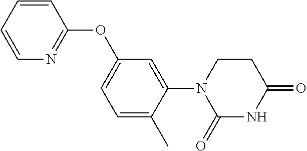 [SMILES: Cc1ccc(Oc2ccccn2)cc1N1CCC(=O)NC1=O]

(26) ¹H NMR (400 MHZ, CDCl₃) δ 8.18 (ddd, J=5.0, 2.0, 0.8 Hz, 1H), 7.69 (ddd, J=8.3, 7.2, 2.0 Hz, 1H), 7.57 (s, 1H), 7.31 (d, J=8.4 Hz, 1H), 7.09-7.04 (m, 1H), 7.03-6.97 (m, 2H), 6.92 (d, J=8.3 Hz, 1H), 3.87-3.75 (m, 1H), 3.68 (q, J=6.2 Hz, 1H), 2.82 (dd, J=6.9, 4.9 Hz, 2H), 2.26 (s, 3H). ¹³C NMR (101 MHZ, CDCl₃) δ 169.4, 163.4, 152.8, 151.1, 147.7, 140.3, 139.6, 132.1, 131.7, 121.2, 119.6, 118.8, 111.7, 45.1, 31.5, 17.4. calcd for [C₁₆H₁₅N₃O₃+H]⁺, 298.1; found, 298.1.

### REFERENCES

(1) Lai, A. C.; Crews, C. M. “Induced Protein Degradation: An Emerging Drug Discovery Paradigm” Nat. Rev. Drug Discovery 2017, 16, 101.
(2) Salami, J.; Crews, C. M. “Waste Disposal—an Attractive Strategy for Cancer Therapy” Science 2017, 355, 1163.
(3) Cromm, P. M.; Crews, C. M. “Targeted Protein Degradation: From Chemical Biology to Drug Discovery” Cell Chem. Biol. 2017, 24, 1181.
(4) Ottis, P.; Crews, C. M. “Proteolysis-Targeting Chimeras: Induced Protein Degradation as a Therapeutic Strategy” ACS Chem. Biol. 2017, 12, 892.
(5) Sakamoto, K. M.; Kim, K. B.; Kumagai, A.; Mercurio, F.; Crews, C. M.; Deshaies, R. J. “Protacs: Chimeric Molecules That Target Proteins to the Skp1-Cullin-F Box Complex for Ubiquitination and Degradation” Proc. Natl. Acad. Sci. USA 2001, 98, 8554.
(6) Sakamoto, K. M.; Kim, K. B.; Verma, R.; Ransick, A.; Stein, B.; Crews, C. M.; Deshaies, R. J. “Development of Protacs to Target Cancer-Promoting Proteins for Ubiquitination and Degradation” Mol. Cell. Proteomics 2003, 2, 1350.
(7) Zhang, D.; Baek, S. H.; Ho, A.; Kim, K. “Degradation of Target Protein in Living Cells by Small-Molecule Proteolysis Inducer” Bioorg. Med. Chem. Lett. 2004, 14, 645.
(8) Winter, G. E.; Buckley, D. L.; Paulk, J.; Roberts, J. M.; Souza, A.; Dhe-Paganon, S.; Bradner, J. E. “Phthalimide Conjugation as a Strategy for in Vivo Target Protein Degradation” Science 2015, 348, 1376.
(9) Lu, J.; Qian, Y. M.; Altieri, M.; Dong, H. Q.; Wang, J.; Raina, K.; Hines, J.; Winkler, J. D.; Crew, A. P.; Coleman, K.; Crews, C. M. “Hijacking the E3 Ubiquitin Ligase Cereblon to Efficiently Target Brd4” Chem. Biol. 2015, 22, 755.
(10) Wang, C.; Zhang, Y. J.; Wu, Y. D.; Xing, D. M. “Developments of Crbn-Based Protacs as Potential Therapeutic Agents” Eur. J. Med. Chem. 2021, 225, 11379.
(11) Bondeson, D. P.; Mares, A.; Smith, I. E. D.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faclth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. “Catalytic in Vivo Protein Knockdown by Small-Molecule Protacs” Nat. Chem. Biol. 2015, 11, 611.
(12) Zengerle, M.; Chan, K. H.; Ciulli, A. “Selective Small Molecule Induced Degradation of the Bet Bromodomain Protein Brd4” ACS Chem. Biol. 2015, 10, 1770.
(13) Wang, C.; Zhang, Y. J.; Wang, J.; Xing, D. M. “Vhl-Based Protacs as Potential Therapeutic Agents: Recent Progress and Perspectives” Eur. J. Med. Chem. 2022, 227, 113906.
(14) Ohoka, N.; Okuhira, K.; Ito, M.; Nagai, K.; Shibata, N.; Hattori, T.; Ujikawa, O.; Shimokawa, K.; Sano, O.; Koyama, R.; Fujita, H.; Teratani, M.; Matsumoto, H.; Imacda, Y.; Nara, H.; Cho, N.; Naito, M. “In Vivo Knockdown of Pathogenic Proteins Via Specific and Nongenetic Inhibitor of Apoptosis Protein (Iap)-Dependent Protein Erasers (Snipers)” J. Biol. Chem. 2017, 292, 4556.
(15) Ma, Z. H.; Ji, Y.; Yu, Y. F.; Liang, D. L. “Specific Non-Genetic Iap-Based Protein Erasers (Snipers) as a Potential Therapeutic Strategy” Eur. J. Med. Chem. 2021, 216, 113247.
(16) Itoh, Y.; Ishikawa, M.; Naito, M.; Hashimoto, Y. “Protein Knockdown Using Methyl Bestatin-Ligand Hybrid Molecules: Design and Synthesis of Inducers of Ubiquitination-Mediated Degradation of Cellular Retinoic Acid-Binding Proteins” J. Am. Chem. Soc. 2010, 132, 5820.
(17) Samarasinghe, K. T. G.; Crews, C. M. “Targeted Protein Degradation: A Promise for Undruggable Proteins” Cell Chem. Biol. 2021, 28, 934.
(18) Burslem, G. M.; Crews, C. M. “Proteolysis-Targeting Chimeras as Therapeutics and Tools for Biological Discovery” Cell 2020, 181, 102.
(19) Burslem, G. M.; Smith, B. E.; Lai, A. C.; Jaime-Figueroa, S.; McQuaid, D. C.; Bondeson, D. P.; Toure, M.; Dong, H. Q.; Qian, Y. M.; Wang, J.; Crew, A. P.; Hines, J.; Crews, C. M. “The Advantages of Targeted Protein Degradation over Inhibition: An Rtk Case Study” Cell Chem. Biol. 2018, 25, 67.
(20) Bondeson, D. P.; Smith, B. E.; Burslem, G. M.; Buhimschi, A. D.; Hines, J.; Jaime-Figueroa, S.; Wang, J.; Hamman, B. D.; Ishchenko, A.; Crews, C. M. “Lessons in Protac Design from Selective Degradation with a Promiscuous Warhead” Cell Chem. Biol. 2018, 25, 78.
(21) Guenette, R. G.; Yang, S. W.; Min, J.; Pei, B.; Potts, P. R. “Target and Tissue Selectivity of Protac Degraders” Chem. Soc. Rev. 2022, in press, DOI: 10.1039/d2cs00200k.
(22) Yang, K.; Song, Y.; Xic, H.; Wu, H.; Wu, Y.-T.; Leisten, E. D.; Tang, W. “Development of the First Small Molecule Histone Deacetylase 6 (Hdac6) Degraders” Bioorg. Med. Chem. Lett. 2018, 28, 2493. (Not supported by NIH)
(23) Mullard, A. “Targeted Protein Degraders Crowd into the Clinic” Nat. Rev. Drug Discovery 2021, 20, 247.
(24) Schapira, M.; Calabrese, M. F.; and, A. N. B.; Crews, C. M. “Targeted Protein Degradation: Expanding the Toolbox” Nat. Rev. Drug Discovery 2019, 18, 949.
(25) Weng, G. Q.; Shen, C.; Cao, D. S.; Gao, J. B.; Dong, X. W.; He, Q. J.; Yang, B.; Li, D.; Wu, J.; Hou, T. J. “Protac-db: An Online Database of Protacs” Nucleic Acids Res. 2021, 49, D1381.
(26) Cecchini, C.; Pannilunghi, S.; Tardy, S.; Scapozza, L. “From Conception to Development: Investigating Protacs Features for Improved Cell Permeability and Successful Protein Degradation” Frontiers in Chemistry 2021, 9, 672267.
(27) Leeson, P. D.; Springthorpe, B. “The Influence of Drug-Like Concepts on Decision-Making in Medicinal Chemistry” Nat. Rev. Drug Discovery 2007, 6, 881.
(28) Doak, B. C.; Over, B.; Giordanetto, F.; Kihlberg, J. “Oral Druggable Space Beyond the Rule of 5: Insights from Drugs and Clinical Candidates” Chem. Biol. 2014, 21, 1115.
(29) Eriksson, T.; Bjorkman, S.; Roth, B.; Fyge, A.; Hoglund, P. “Stereospecific Determination, Chiral Inversion in-Vitro and Pharmacokinetics in Humans of the Enantiomers of Thalidomide” Chirality 1995, 7, 44.
(30) Chamberlain, P. P.; Lopez-Girona, A.; Miller, K.; Carmel, G.; Pagarigan, B.; Chie-Leon, B.; Rychak, E.; Corral, L. G.; Ren, Y. J.; Wang, M.; Riley, M.; Delker, S. L.; Ito, T.; Ando, H.; Mori, T.; Hirano, Y.; Handa, H.; Hakoshima, T.; Daniel, T. O.; Cathers, B. E. “Structure of the Human Cereblon-Ddb1-Lenalidomide Complex Reveals Basis for Responsiveness to Thalidomide Analogs” Nat. Struct. Mol. Biol. 2014, 21, 803.
(31) Mori, T.; Ito, T.; Liu, S. J.; Ando, H.; Sakamoto, S.; Yamaguchi, Y.; Tokunaga, E.; Shibata, N.; Handa, H.; Hakoshima, T. “Structural Basis of Thalidomide Enantiomer Binding to Cereblon” Sci. Rep. 2018, 8.
(32) Fischer, E. S.; Bohm, K.; Lydcard, J. R.; Yang, H. D.; Stadler, M. B.; Cavadini, S.; Nagel, J.; Serluca, F.; Acker, V.; Lingaraju, G. M.; Tichkule, R. B.; Schebesta, M.; Forrester, W. C.; Schirle, M.; Hassiepen, U.; Ottl, J.; Hild, M.; Beckwith, R. E. J.; Harper, J. W.; Jenkins, J. L.; Thoma, N. H. “Structure of the Ddbi-Crbn E3 Ubiquitin Ligase in Complex with Thalidomide” Nature 2014, 512, 49.
(33) “Fda's Policy Statement for the Development of New Stereoisomeric Drugs” Chirality 1992, 4, 338.
(34) Kazantsev, A.; Krasavin, M. “Ligands for Cereblon: 2017-2021 Patent Overview” Expert Opin. Ther. Pat. 2022, 32, 171.
(35) Jacques, V.; Czarnik, A. W.; Judge, T. M.; Van der Ploeg, L. H. T.; DeWitt, S. H. “Differentiation of Antiinflammatory and Antitumorigenic Properties of Stabilized Enantiomers of Thalidomide Analogs” Proc. Natl. Acad. Sci. USA 2015, 112, E1471.
(36) Hartmann, M. D.; Boichenko, I.; Coles, M.; Zanini, F.; Lupas, A. N.; Alvarez, B. H. “Thalidomide Mimics Uridine Binding to an Aromatic Cage in Cereblon” J. Struct. Biol. 2014, 188, 225.
(37) Boichenko, I.; Bar, K.; Deiss, S.; Heim, C.; Albrecht, R.; Lupas, A. N.; Alvarez, B. H.; Hartmann, M. D. “Chemical Ligand Space of Cereblon” Acs Omega 2018, 3, 11163.
(38) Sosic, I.; Bricelj, A.; Steinebach, C. “E3 Ligase Ligand Chemistries: From Building Blocks to Protein Degraders” Chem. Soc. Rev. 2022, 51, 3487.
(39) Yamamoto, T.; Tokunaga, E.; Nakamura, S.; Shibata, N.; Toru, T. “Synthesis and Configurational Stability of (S)- and (R)-Deuteriothalidomides” Chem. Pharm. Bull. 2010, 58, 110.
(40) Nishimura, K.; Hashimoto, Y.; Iwasaki, S. “(S)-Form of Alpha-Methyl-N(Alpha)-Phthalimidoglutarimide, but Not Its (R)-Form, Enhanced Phorbol Ester-Induced Tumor-Necrosis-Factor-Alpha Production by Human Leukemia-Cell Hl-60—Implication of Optical Resolution of Thalidomidal Effects” Chem. Pharm. Bull. 1994, 42, 1157.
(41) Hansen, J. D.; Correa, M.; Nagy, M. A.; Alexander, M.; Plantevin, V.; Grant, V.; Whitefield, B.; Huang, D. H.; Kercher, T.; Harris, R.; Narla, R. K.; Leisten, J.; Tang, Y.; Moghaddam, M.; Ebinger, K.; Piccotti, J.; Havens, C. G.; Cathers, B.; Carmichael, J.; Daniel, T.; Vessey, R.; Hamann, L. G.; Leftheris, K.; Mendy, D.; Baculi, F.; LeBrun, L. A.; Khambatta, G.; Lopez-Girona, A. “Discovery of Crbn E3 Ligase Modulator Cc-92480 for the Treatment of Relapsed and Refractory Multiple Myeloma” J. Med. Chem. 2020, 63, 6648.
(42) Fromme, J. C.; Verdine, G. L. “DNA Lesion Recognition by the Bacterial Repair Enzyme Mutm” J. Biol. Chem. 2003, 278, 51543.
(43) Barreiro, E. J.; Kummerle, A. E.; Fraga, C. A. M. “The Methylation Effect in Medicinal Chemistry” Chem. Rev. 2011, 111, 5215.
(44) Leung, C. S.; Leung, S. S. F.; Tirado-Rives, J.; Jorgensen, W. L. “Methyl Effects on Protein-Ligand Binding” J. Med. Chem. 2012, 55, 4489.
(45) Min, J.; Mayasundari, A.; Keramatnia, F.; Jonchere, B.; Yang, S. W.; Jarusiewicz, J.; Actis, M.; Das, S.; Young, B.; Slavish, J.; Yang, L.; Li, Y.; Fu, X.; Garrett, S. H.; Yun, M. K.; Li, Z. M.; Nithianantham, S.; Chai, S.; Chen, T. S.; Shelat, A.; Lee, R. E.; Nishiguchi, G.; White, S. W.; Roussel, M. F.; Potts, P. R.; Fischer, M.; Rankovic, Z. “Phenyl-Glutarimides: Alternative Cereblon Binders for the Design of Protacs” Angew. Chem. Int. Ed. 2021, 60, 26663.
(46) Nowak, R. P.; DeAngelo, S. L.; Buckley, D.; He, Z. X.; Donovan, K. A.; An, J.; Saface, N.; Jedrychowski, M. P.; Ponthier, C. M.; Ishoey, M.; Zhang, T. H.; Mancias, J. D.; Gray, N. S.; Bradner, J. E.; Fischer, E. S. “Plasticity in Binding Confers Selectivity in Ligand-Induced Protein Degradation” Nat. Chem. Biol. 2018, 14, 706.
(47) Douglass, E. F.; Miller, C. J.; Sparer, G.; Shapiro, H.; Spiegel, D. A. “A Comprehensive Mathematical Model for Three-Body Binding Equilibria” J. Am. Chem. Soc. 2013, 135, 6092.
(48) Nawrocki, S. T.; Griffin, P.; Kelly, K. R.; Carew, J. S. “Mln4924: A Novel First-in-Class Inhibitor of Nedd8-Activating Enzyme for Cancer Therapy” Expert Opinion on Investigational Drugs 2012, 21, 1563.

What is claimed is:

1. A compound that binds cereblon, having a structure as shown in Formula I: 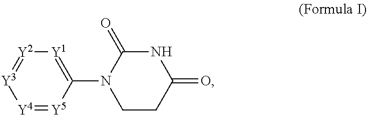
wherein:
Y¹, Y², Y³, Y⁴, and Y⁵ are each independently CH, CR, CR¹, or N, with the proviso that at least one of Y¹, Y², Y³, Y⁴, and Y⁵ is CR;
R¹ in each instance is independently C1-C3 alkyl, halogen-substituted C1-C3 alkyl, C1-C3 alkyloxy, or halogen-substituted C1-C3 alkyloxy; and
R in each instance is independently hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

2. The compound of claim 1, wherein:
Y¹ and Y³ are each independently CH, CR, CR¹, or N; and
Y², Y⁴, and Y⁵ are each independently CH, CR, or CR¹.

3. The compound of claim 1, wherein:
Y¹ is CH or CR¹;
Y², Y³, and Y⁴ are each independently CH or CR; and
and Y⁵ is CH.

4. The compound of claim 1, wherein;
Y¹ is CH or CR¹;
Y² and Y⁴ are each independently CH or CR; and
Y³ and Y⁵ is CH.

5. The compound of claim 4, wherein Y¹ is CR¹.

6. The compound of claim 5, wherein Y² is CR.

7. The compound of claim 5, wherein Y⁴ is CR.

8. The compound of claim 1, wherein:
R is: 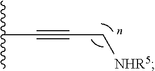
n is 1-4;
R⁵ is an amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

9. The compound of claim 1, wherein:
R is: 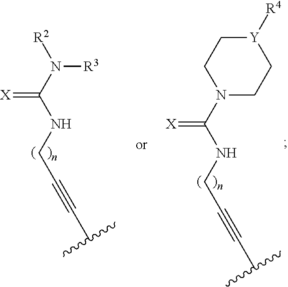
n is 1-4;
X is O or S;
R² is R^{A};
R³ is an amino protecting group or R^{A};
Y is N or CH;
R⁴ is amino protecting group or R^{A}; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

10. The compound of claim 1, wherein:
R is —X—R^{A};
X is —O—, NH, or —C(O)—; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

11. The compound of claim 1, wherein:
R is: 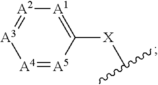
X is —O—, NH, or —C(O)—;
A¹, A², A³, A⁴, and A⁵ are each independently CH, CE, CR^{A}, or N, with the proviso that at least one of A¹, A², A³, A⁴, and A⁵ is CR^{A};
E is halogen, halogen-substituted alkyl, alkyloxy, alkenyloxy, alkynyloxy, cycloalkyloxy, cycloalkenyloxy, alkylsulfonyloxy, cycloalkenylsulfonyloxy, amino, protected amino, acyl, formyl, alkyloxycarbonyl, alkenyloxycarbonyl, alkynyloxycarbonyl, aryloxycarbonyl, cyano, nitro, aryloxy, heteroaryloxy, heteroarylsulfonyloxy, non-aromatic heterocycle, or any combination of the forgoing; and
R^{A} is hydroxyl, halogen, alkyl, alkenyl, alkynyl, cycloalkyl, cycloalkenyl, carboxyl, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, protected amino, acyl, formyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, non-aromatic heterocycle, or any combination of the forgoing.

12. The compound of claim 11, wherein one or two of A¹, A², A³, A⁴, and A⁵ is N.

13. The compound of claim 11, wherein:
at least one of A², A³, and A⁴ is CR^{A}; and
at least one of A², A³, and A⁴ is CE.

14. The compound of claim 13, wherein A¹ and A⁵ are each CH.

15. The compound of claim 1, wherein R is selected from:

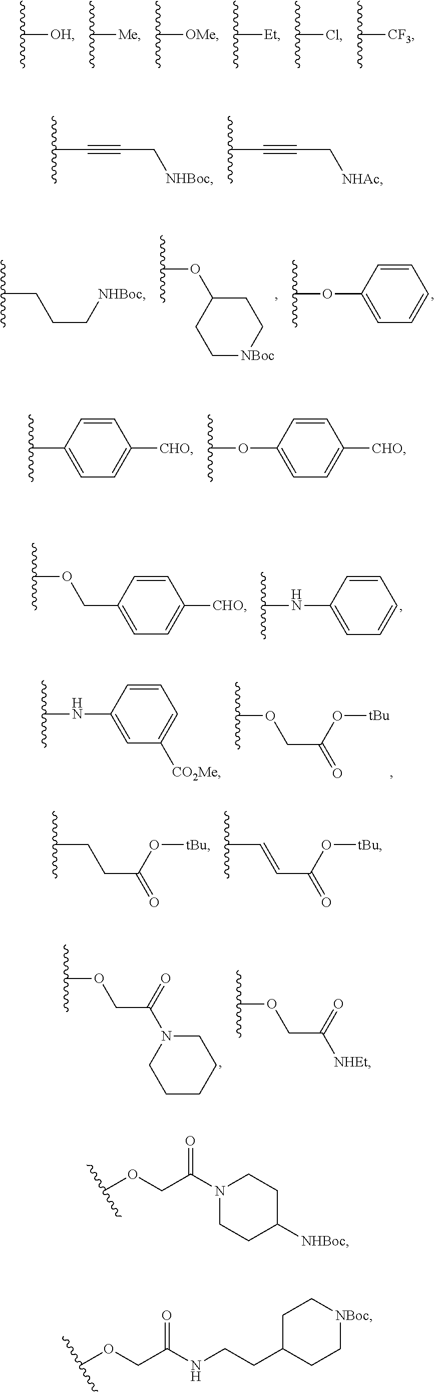

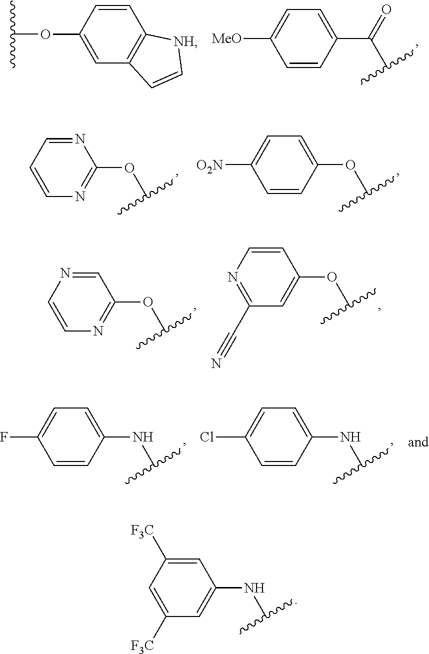

16. A proteolysis targeting chimera (PROTAC), comprising the compound of claim 1 configured to bind cereblon and a protein binder configured to bind a target protein.

17. The PROTAC of claim 16, wherein the protein binder is a polypeptide, a ligand, an aptamer, a nanoparticle, or a small molecule.

18. The PROTAC of claim 16, further comprising a linker connecting the compound of claim 1 and the protein binder.

19. A method of degrading a target protein, comprising contacting the target protein with the PROTAC of claim 16, wherein the PROTAC mediates degradation of the target protein in a proteasome.
